(19) United States
(12) Patent Application Publication
(10) Pub. No.: US 2023/0172894 A1
(43) Pub. Date: Jun. 8, 2023

(54) COMBINATION TREATMENT FOR FUMARATE-RELATED DISEASES

(71) Applicant: IMCYSE SA, Angleur (Liège) (BE)

(72) Inventors: Jean-Marie SAINT-REMY, Grez-Doiceau (BE); Luc VANDER ELST, Obaix (BE); Vincent CARLIER, Enines (BE); Milos ERAK, Brussels (BE); Jean VAN RAMPELBERGH, Brussels (BE); Marcelle VAN MECHELEN, Villers-Perwin (BE); David WALGRAFFE, Hannut (BE); Geoffrey GLOIRE, Liège (BE); Jean SMAL, Schaerbeek (BE)

(21) Appl. No.: 17/923,215

(22) PCT Filed: May 6, 2021
(86) PCT No.: PCT/EP2021/061972

§ 371 Date: Nov. 3, 2022

(30) Foreign Application Priority Data
May 6, 2020 (EP) 20173209.6
Jun. 9, 2020 (EP) 20179019.3

(51) Int. Cl. A61K 31/225; A61K 31/4015; C07K 14/705; A61P 25/28

(52) U.S. Cl. CPC A61K 31/225; A61K 31/4015; C07K 14/70503; A61P 25/28

(57) ABSTRACT

The present invention relates to a pharmaceutical preparation (combination or pharmaceutical composition or kit-of-parts) comprising one or more dose units of a fumarate compound and an immunogenic or tolerogenic peptide comprising an oxidoreductase motif and an NKT cell epitope or an MHC class II T cell epitope of an (auto)antigen involved in fumarate related diseases or disorders. The present invention further relates to medical uses of this pharmaceutical preparation.

### FIELD OF THE INVENTION

The present invention relates to a pharmaceutical preparation (combination or pharmaceutical composition or kit-of-parts) comprising a fumarate-based component and an immunogenic peptide comprising an oxidoreductase motif and an NKT cell epitope or an MHC class II T cell epitope of an auto-antigen involved in a disease capable of being treated with said fumarate-based component or a tolerogenic peptide comprising an NKT cell epitope or an MHC class II T cell epitope. The present invention further relates to medical uses of this pharmaceutical preparation.

### BACKGROUND OF THE INVENTION

Fumarate compounds such as monomethylfumarate or its prodrug dimethylfumarate have been implemented in several diseases, going from de-myelinating disorders, over cancer to transplantation rejection. Fumarate or fumaric acid is the hydrolysis product of monomethylfumarate (MMF), which in turn is the hydrolysis product of dimethylfumarate (DMF). Fumarate as such has been reported to be implicated in the succination of cysteine residues in certain proteins such as KEAP1, CTSZ, GAPDH, MGST3, NUBP1, PRDX1 & 3, TXN, and UCHL1, thereby hampering their functionality and leading to a defect. DMF is converted into its active metabolite MMF, which binds to Nrf2. Subsequently, Nrf2 translocates to the nucleus and binds to the antioxidant response element (ARE). This induces the expression of a number of cytoprotective genes, including NAD(P)H quinone oxidoreductase 1 (NQO1), sulfiredoxin 1 (Srxn1), heme oxygenase-1 (HO1, HMOX1), superoxide dismutase 1 (SOD1), gamma-glutamylcysteine synthetase (gamma-GCS), thioredoxin reductase-1 (TXNRD1), glutathione S-transferase (GST), glutamate-cysteine ligase catalytic subunit (Gclc) and glutamate-cysteine ligase regulatory subunit (Gclm); this also increases the synthesis of the antioxidant glutathione (GSH). The intraneuronal synthesis of GSH may protect neuronal cells from damage due to oxidative stress. DMF also appears to inhibit the nuclear factor-kappa B (NF-kB)-mediated pathway, modulates the production of certain cytokines and induces apoptosis in certain T-cell subsets. Its radiosensitizing activity is due to this agent's ability to bind to and sequester intracellular GSH, thereby depleting intracellular GSH and preventing its anti-oxidative effects. This enhances the cytotoxicity of ionizing radiation in hypoxic cancer cells. Nrf2, a leucine zipper transcription factor, plays a key role in redox homeostasis and cytoprotection against oxidative stress.

DMF is currently being investigated in a number of diseases such as: Multiple Sclerosis (MS), Neuromyelitis Optica (NMO), psoriasis, Rheumatoid Arthritis (RA), asthma, atopic dermatitis, scleroderma, ulcerative colitis, cancer and transplantation rejection.

WO2008017517 describes a technology allowing polarization of CD4+ T cells into a cytolytic phenotype, thereby allowing them to induce apoptosis of APCs after peptide-MHC class II cognate recognition, and hence suppressing the immune response against a specific antigen. This can be achieved by increasing the strength of the synapse created with peptide-MHC complexes thanks to the addition of an oxidoreductase motif within the flanking residues of class II-restricted epitopes. This technology can also prevent or suppress immune response to multiple antigens, because APC apoptosis can also prevent activation of CD4+ T cells to alternative epitopes of the autoantigen from which the peptide is derived or to epitopes of associated autoantigens. Moreover, CD4+ T cells polarized into a cytolytic phenotype eliminate by apoptosis bystander CD4+ T cells provided they are activated at the surface of the same APC. Finally, WO2008017517 discloses that cytolytic CD4+ T cells generated using the above technology have a memory phenotype, thereby allowing a long-term functionality.

WO2008/017517 demonstrates this concept for allergies and auto-immune diseases such as type 1 diabetes, where insulin can act as an auto-antigen. These immunogenic peptides are used either by direct vaccination or for in vitro conversion of CD4+ T cells into cytolytic CD4+ cells.

The WO2012069568 patent application describes the same concept but with cytolytic conversion of CD1d-restricted NKT cells thanks to the use of CD1d-restricted peptide epitopes fused to an oxidoreductase motif. These immunogenic peptides are used either by direct vaccination or for in vitro conversion of CD1d-restricted NKT cells.

WO2017182528 describes the use of an immunogenic peptide comprising a Myelin Oligodendrocyte Glycoprotein (MOG) epitope for use in treating Multiple Sclerosis.

In addition, tolerogenic peptides comprising T cells epitopes have been used for inducing tolerance towards certain auto- or self-antigens. Patent application WO0216410 for example describes antigen processing independent epitopes that are of an appropriate size to be presented by immature APC without antigen processing, which would favour immunological tolerance. WO2018182495 further discloses tolerogenic peptides comprising T cell epitopes to treat multiple sclerosis.

In search for an improved treatment method of the above indicated diseases and disorders, the present invention provides a synergistic combination treatment of a fumarate compound and either a tolerogenic peptide, or an immunogenic peptide comprising an oxidoreductase motif.

### SUMMARY OF THE INVENTION

The invention hence provides the following aspects:

Aspect 1. A pharmaceutical kit comprising:

a) one or more dosage forms of a fumarate compound of the general formula (I)

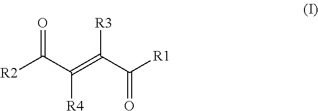

wherein R¹ and R² each independently are selected from the groups consisting of: OH, O⁻, and optionally substituted (C₁₋₁₀)alkoxy, preferably optionally substituted (C₁₋₆)alkoxy, or optionally substituted (C₁₋₃)alkoxy,
wherein R³ and R⁴ each independently are selected from the groups consisting of: H or deuterium,
wherein each group independently can be optionally substituted as outlined herein elsewhere; and

b) one or more dosage forms of an immunogenic or tolerogenic peptide comprising, consisting, or consisting essentially of, a T cell epitope of an antigenic protein involved in a fumarate-related disease or disorder, more preferably a T cell epitope capable of binding to an MHC class I or II molecule or an NKT cell epitope of an antigenic protein involved in a fumarate-related disease or disorder.

Aspect 2. The pharmaceutical kit according to aspect 1, wherein said peptide is an immunogenic peptide having an oxidoreductase motif linked to said T cell epitope, said oxidoreductase motif having a sequence of the general formula:

Zₘ-[CST]-Xₙ-C- (SEQ ID NO: 1 to 25) or Zₘ-C-Xₙ-[CST]- (SEQ ID NO: 26 to 50),

wherein n is an integer chosen from 0 to 6, preferably wherein n is 2, 1, 3, or 0, wherein m is an integer selected from 0 to 3, wherein X is any amino acid, wherein Z is any amino acid, in which [CST] stands for any one of cysteine (C), serine (S), or threonine (T);
wherein said oxidoreductase motif and said T cell epitope are separated by a linker of between 0 and 7 amino acids,
wherein the hyphen (-) in said oxidoreductase motif indicates the point of attachment of the oxidoreductase motif to the N-terminal end of the linker or the epitope, or to the C-terminal end of the linker or the T cell epitope.

In some embodiments, the fumarate compound is a deuterated form of any of the foregoing fumarate compounds, or a clathrate, a solvate, a tautomer, a stereoisomer, or a non-toxic, pharmaceutically acceptable salt thereof such as an acid addition salt, or a combination of any of the foregoing.

In some embodiments, the pharmaceutically acceptable salt is a salt of a metal (M) cation, wherein M can be an alkali, alkaline earth, or transition metal such as Li, Na, K, Ca, Zn, Sr, Mg, Fe, or Mn.

In a preferred embodiment, said oxidoreductase motif is not part of a repeat of the standard C-XX-[CST] or [CST]-XX-C oxidoreductase motifs such as repeats of said motif which can be spaced from each other by one or more amino acids (e.g. CXXC X CXXC X CXXC (SEQ ID NO: 196)), as repeats which are adjacent to each other (CXXCCXXCCXXC (SEQ ID NO: 197)) or as repeats which overlap with each other CXXCXXCXXC (SEQ ID NO: 198) or CXCCXCCXCC (SEQ ID NO: 199)), especially when n is 0 or 1 and m is 0 in the general formula as defined in aspect 2.

Aspect 3. The pharmaceutical kit according to aspect 1 or 2, wherein said fumarate compound is selected from the group consisting of: dialkyl fumarate, monoalkyl fumarate, a combination of a dialkyl fumarate and a monoalkyl fumarate, such as a combination of dimethyl fumarate and monomethyl fumarate, or a combination of any of the foregoing.

Aspect 4. The pharmaceutical kit according to aspect 1, 2 or 3, wherein said fumarate compound is dimethyl fumarate—DMF (R1 is OCH3 and R2 is OCH3—Formula (II) below), or monomethyl fumarate—MMF (R1 is OCH3 and R2 is O— or OH—Formula (III) below), or a combination thereof, or a deuterated form, a clathrate, a solvate, a tautomer, a stereoisomer, or a pharmaceutically acceptable salt thereof.

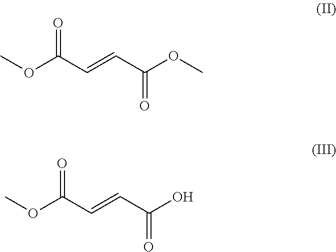

In preferred embodiments of said fumarate compound of Formula (I), said

(C₁₋₁₀)alkoxy group in R¹ or R² can be chosen from: ethoxy, methoxy,

(C₁₋₅)alkoxy, (C₁₋₄)alkoxy, (C₁₋₃)alkoxy, (C₂₋₃)alkoxy,

(C₂₋₄)alkoxy, (C₂₋₅) alkoxy, and (C₁₋₆)alkoxy.

Preferred examples of said fumarate compounds are prodrugs of monoalkylfumarate or more specifically monomethylfumarate, i.e. compounds that can be metabolized into monomethyl fumarate in vivo, such as those compounds of Formula (I) wherein R¹ is C₁-C₃ alkoxy such as methoxy, ethoxy or propoxy and wherein R² is C₁-C₃ alkoxy such as methoxy, ethoxy or propoxy which is optionally substituted.

Further preferred examples are those wherein R¹ is methoxy and R² is methoxy or optionally substituted ethoxy.

In some embodiments, the fumarate compound is a prodrug of monoalkyl fumarate such as diroximel fumarate (formula (IV)):

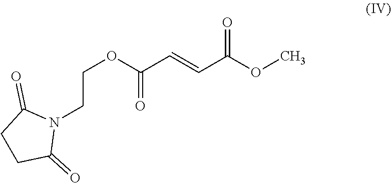 [SMILES: COC(=O)/C=C/C(=O)OCCN1C(=O)CCC1=O]

or tepilamide fumarate (formula (V)):

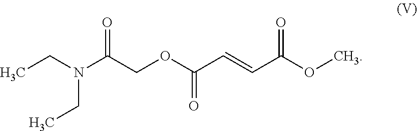 [SMILES: CCN(CC)C(=O)COC(=O)/C=C/C(=O)OC]

Further examples of fumarate compounds that could be used in combination with the immunogenic or tolerogenic peptides as disclosed herein are discussed below.

In further illustrative embodiments, the fumarate compound is a calcium salt of MMF (Ca-MMF) or DMF (Ca-DMF), optionally in a deuterated form, wherein one or more of the alkyl groups is a deuterated alkyl group, such as a deuterated methyl group that contains at least one deuterium atom. Examples of deuterated methyl include: —CDH2, —CD2H, and —CD3. Examples of deuterated ethyl include: —CHDCH3, —CD2CH3, —CHDCDH2,

—CHDCD2H, CHDCD3, —CD2CDH2, CD2CD2H, and CD2CD3.

Aspect 5. The pharmaceutical kit according to any one of aspects 1 to 4, wherein said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen.

Aspect 6. The pharmaceutical kit according to any one of aspects 1 to 5, wherein said fumarate-related disease or disorder is an auto-immune disorder, a demyelinating disorder, transplant rejection or cancer, preferably a demyelinating disorder.

Preferred examples of fumarate-related diseases and disorders of the auto-immune type are: Multiple Sclerosis (MS), Neuromyelitis optica (NMO), preferably MOG-induced NMO (i.e. MO caused by anti-MOG antibodies or MOG autoantigens), psoriasis, Rheumatoid Arthritis (RA), polyarthritis, asthma, atopic dermatitis, scleroderma, ulcerative colitis, juveline diabetes, thyreoiditis, Grave's disease, Systemic Lupus Erythromatosis (SLE), Sjögren syndrome, anemia perniciosa, chronic active hepatitis, transplant rejection and cancer.

In some embodiments, said demyelinating disorder is selected from: Multiple Sclerosis (MS), Neuromyelitis Optica (NMO), Optic Neuritis, Acute Disseminated Encephalomyelitis, Balo's Disease, HTLV-1 Associated Myelopathy, Schilder's Disease, Transverse Myelitis, Idiopathic inflammatory demyelinating diseases, vitamin B12-induced central nervous system neuropathies, Central pontine myelinolysis, Myelopathies including tabes dorsalis, Leukodystrophies such as Adrenoleukodystrophy, Leukoencephalopathies such as Progressive multifocal leukoencephalopathy (PML), Vanishing White Matter Disease, and Rubella induced mental retardation.

In preferred embodiments, the demyelinating disorder is caused or aggravated by MOG auto-antigens and/or anti-MOG antibodies and hence selected from the group consisting of: Multiple Sclerosis (MS), Neuromyelitis Optica (NMO), Optic Neuritis, Acute Disseminated Encephalomyelitis, Transverse Myelitis, Adrenoleukodystrophy, Vanishing White Matter Disease, and Rubella induced mental retardation. In more preferred embodiments the demyelinating disorder is Multiple Sclerosis (MS) or Neuromyelitis Optica (NMO). In certain embodiments, said MS is selected from Clinically Isolated Syndrome (CIS), relapse-remitting MS (RRMS), secondary progressive MS (SPMS), primary progressive MS (PPMS), Acute Fulminant Multiple Sclerosis and MS-suspected radiology isolated syndrome (RIS).

Aspect 7. The pharmaceutical kit according to any one of aspects 1 to 6, wherein said fumarate-related disease or disorder is MS and wherein said autoantigen is selected from the group consisting of: Myelin Oligodendrocyte Glycoprotein (MOG), Myelin basic protein (MBP), Proteolipid protein (PLP), myelin-associated antigen (MAG), Oligodendrocyte-specific protein (OSP), myelin-associated oligodendrocyte basic protein (MOBP), 2′,3′-cyclic-nucleotide 3′-phosphodiesterase (CNPase), S100β protein and transaldolase H, preferably MOG; or wherein said fumarate-related disease or disorder is a MOG autoantigen induced disease or disorder, preferably MS or NMO, wherein said antigenic protein is MOG.

Aspect 8. The pharmaceutical kit according to any one of aspects 1 to 7, wherein said fumarate-related disease or disorder is Rheumatoid Arthritis (RA) and wherein said antigenic protein is selected from the group comprising: GRP78, HSP60, 60 kDa chaperonin 2, Gelsolin, Chitinase-3-like protein 1, Cathepsin S, Serum albumin, vinculin, and Cathepsin D.

Aspect 9. The pharmaceutical kit according to any one of aspects 1 to 8, wherein said fumarate-related disease or disorder is Psoriasis and wherein said antigenic protein is selected from the group consisting of: ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37, preferably LL37.

Aspect 10. The pharmaceutical kit according to any one of aspects 1 to 9, wherein the said (auto)antigen involved in a fumarate-related disease or disorder does not naturally comprise an oxidoreductase motif within 11 amino acids N- or C-terminally adjacent to said epitope.

Aspect 11. The pharmaceutical kit according to aspect 10, wherein in said immunogenic peptide said epitope does not naturally comprise an oxidoreductase motif in its sequence.

Aspect 12. The pharmaceutical kit according to any one of aspects 1 to 11, wherein in said immunogenic or tolerogenic peptide the T-cell epitope is an MHC class I or II T-cell epitope or an NKT cell epitope.
An MHC class II epitope typically has a length of between 7 and 20 amino acids in length, more usually between 8 and 20 or 9 and 20 amino acids in length, even more preferably between 7 and 17, between 8 and 17, between 9 and 17, between 10 and 17, between 11 and 17, between 12 and 17, between 13 and 17 amino acids, such as between 14 and 16 amino acids. Peptides which bind to MHC class II molecules can also be longer since these peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.
An MHC class I T-cell epitope typically has a length of between 7 to 13, more preferably between 8 to 10 amino acids in length. The binding of the peptide is stabilized at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues which allow flexibility of the chain;
An NKT cell epitope can be recognized and bound by a receptor at the cell surface of an NKT cell, in particular by CD1d molecules. Such an epitope typically has a length of between 7 and 20 amino acids, more usually between 7 and 17 amino acids in length, even more preferably between 8 and 17, between 9 and 17, between 10 and 17, between 11 and 17, between 12 and 17, between 13 and 17 amino acids, such as between 14 and 16 amino acids. Such epitopes typically have a motif [FWHY]-XX-[ILMV]-XX-[FWTHY] [SEQ ID NO: 51] or [FW]-XX-[ILMV]-XX-[FW] [SEQ ID NO: 52].

Aspect 13. The pharmaceutical kit according to any one of aspects 1 to 12, wherein said T-cell epitope is an immunodominant epitope, a subdominant epitope, a cryptic epitope or a minor epitope, preferably an immunodominant or subdominant epitope, more preferably an immunodominant epitope.

Aspect 14. The pharmaceutical kit according to any one of aspects 2 to 13, wherein in said immunogenic peptide the oxidoreductase motif is located N-terminally from the linker or the epitope, or C-terminally from the linker or the epitope, preferably N-terminally from the linker or the epitope, and/or wherein the oxidoreductase motif is located at the N-terminal or C-terminal end of the immunogenic peptide, preferably wherein Z corresponds to the N- or C-terminal end of the immunogenic peptide.

Aspect 15: The pharmaceutical kit according to any one of aspects 1 to 14, wherein in said immunogenic or tolerogenic peptide said T cell epitope of an antigenic protein is an NKT cell epitope or an MHC class II T cell epitope, preferably wherein when said T cell epitope of an antigenic protein is an NKT cell epitope, it has a length of between 7 and 25 amino acids; or wherein when said T cell epitope of an antigenic protein is an MHC class II T cell epitope, it has a length of between 9 and 25 amino acids.

Aspect 16: The pharmaceutical kit according to any one of aspects 1 to 15, wherein said immunogenic or tolerogenic peptide has a length of between 7 and 50 amino acids, and/or wherein said immunogenic or tolerogenic peptide comprising an MHC class II T cell epitope has a length of between 9 and 50 amino acids.

Aspect 17. The pharmaceutical kit according to any one of aspects 2 to 16, wherein in said immunogenic peptide the linker between the oxidoreductase motif and the T cell epitope is of between 0 and 4 amino acids.

Aspect 18. The pharmaceutical kit according to any one of aspects 2 to 17, wherein in said immunogenic peptide said oxidoreductase motif with the sequence Zₘ-[CST]-Xₙ-C- or Zₘ-C-Xₙ-[CST]- as defined in aspect 2, is selected from the following amino acid motifs:

(a) Zₘ-[CST]-Xₙ-C- or Zₘ-C-Xₙ-[CST]- as defined in aspect 2, wherein n is 0, and:

wherein m is an integer selected from 0 to 3,
wherein Z is any amino acid, preferably a basic amino acid selected from: H, K, R, and a non-natural basic amino acid as defined herein, such as L-ornithine, preferably K or H.

In preferred embodiments of motif (a), m is 1 or 2, and Z is a basic amino acid selected from: H, K, R, and a non-natural basic amino acid as defined herein, such as L-ornithine, preferably K or H.

Particularly preferred but non-limiting examples of such motifs are CC, KCC, KKCC (SEQ ID NO: 53), RCC, RRCC (SEQ ID NO: 54), RKCC (SEQ ID NO: 55), or KRCC (SEQ ID NO: 56);

(b) Zₘ-[CST]-Xₙ-C- or Zₘ-C-Xₙ-[CST]- as defined in aspect 2, wherein n is 1,

wherein X is any amino acid, preferably a basic amino acid selected from: H, K, R, and a non-natural basic amino acid such as L-ornithine, preferably K or R, most preferably R,
wherein m is an integer selected from 0 to 3,
wherein Z is any amino acid, preferably a basic amino acid selected from: H, K, R, and a non-natural basic amino acid as defined herein, such as L-ornithine, preferably K or H.

In preferred embodiments of motif (b), m is 1 or 2 and Z is a basic amino acid selected from: H, K, R, and a non-natural basic amino acid as defined herein, such as L-ornithine, preferably K or H.

Particularly preferred but non-limiting examples of such motifs are CRC, CKC, KCXC (SEQ ID NO: 57), KKCXC (SEQ ID NO: 58), RCXC (SEQ ID NO: 59), RRCXC (SEQ ID NO: 60), RKCXC (SEQ ID NO: 61), KRCXC (SEQ ID NO: 52), KCKC (SEQ ID NO: 63), KKCKC (SEQ ID NO: 64), KCRC (SEQ ID NO: 65), KKCRC (SEQ ID NO: 66), RCRC (SEQ ID NO: 67), RRCRC (SEQ ID NO: 68), RKCKC (SEQ ID NO: 69), or KRCKC (SEQ ID NO: 70);

(c) Zₘ-[CST]-Xₙ-C- or Zₘ-C-Xₙ-[CST]- as defined in aspect 2, wherein n is 2, thereby creating an internal X¹X² amino acid couple within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2.

In preferred embodiments, m is 1 and Z is a basic amino acid selected from: H, K, or R, or a non-natural basic amino acid as defined herein, such as L-ornithine, preferably K or H.

In preferred embodiments X¹ and X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a specific embodiment, at least one of X¹ or X² in said motif is a basic amino acid selected from: H, K, or R, or a non-natural basic amino acid as defined herein, such as L-ornithine. In another specific embodiment, at least one of X¹ or X² in said motif is P or Y. Specific examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH.

Particularly preferred motifs of this type are HCPYC (SEQ ID NO: 71), KCPYC (SEQ ID NO: 72), RCPYC (SEQ ID NO: 73), HCGHC (SEQ ID NO: 74), KCGHC (SEQ ID NO: 75), RCGHC (SEQ ID NO: 76), KHCPYC (SEQ ID NO: 77), KKCPYC (SEQ ID NO: 78), KRCPYC (SEQ ID NO: 79), KHCGHC (SEQ ID NO: 80), KKCGHC (SEQ ID NO: 81), and KRCGHC (SEQ ID NO:82);

(d) Zₘ-[CST]-Xₙ-C- or Zₘ-C-Xₙ-[CST]- as defined in aspect 2, wherein n is 3, thereby creating an internal X¹X2X³ amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2.

In some embodiments, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine.

Specific examples of the internal X¹X²X³ amino acid stretch within the oxidoreductase motif are: XPY, PXY, and PYX, wherein X can be any amino acid, preferably a basic amino acid such as K, R, or H, or a non-natural basic amino acid such as L-ornithine.

Non-limiting examples are:

KPY, RPY, HPY, GPY, APY, VPY, LPY, IPY, MPY, FPY, WPY, PPY, SPY, TPY, CPY, YPY, NPY, QPY, DPY, EPY, and KPY; or

PKY, PRY, PHY, PGY, PAY, PVY, PLY, PlY, PMY, PFY, PWY, PPY, PSY, PTY, PCY, PYY, PNY, PQY, PDY, PEY, and PLY; or

PYK, PYR, PYH, PYG, PYA, PYV, PYL, PYI, PYM, PYF, PYW, PYP, PYS, PYT, PYC, PYY, PYN, PYQ, PYD, PYE, and PYL;

XHG, HXG, and HGX, wherein X can be any amino acid, such as in:

KHG, RHG, HHG, GHG, AHG, VHG, LHG, IHG, MHG, FHG, WHG, PHG, SHG, THG, CHG, YHG, NHG, QHG, DHG, EHG, and KHG; or

HKG, HRG, HHG, HGG, HAG, HVG, HLG, HIG, HMG, HFG, HWG, HPG, HSG, HTG, HCG, HYG, HNG, HQG, HDG, HEG, and HLG; or

HGK, HGR, HGH, HGG, HGA, HGV, HGL, HGI, HGM, HGF, HGW, HGP, HGS, HGT, HGC, HGY, HGN, HGQ, HGD, HGE, and HGL;

XGP, GXP, and GPX, wherein X can be any amino acid, such as in:

KGP, RGP, HGP, GGP, AGP, VGP, LGP, IGP, MGP, FGP, WGP, PGP, SGP, TGP, CGP, YGP, NGP, QGP, DGP, EGP, and KGP; or

GKP, GRP, GHP, GGP, GAP, GVP, GLP, GIP, GMP, GFP, GWP, GPP, GSP, GTP, GCP, GYP, GNP, GQP, GDP, GEP, and GLP; or

GPK, GPR, GPH, GPG, GPA, GPV, GPL, GPI, GPM, GPF, GPW, GPP, GPS, GPT, GPC, GPY, GPN, GPQ, GPD, GPE, and GPL;

XGH, GXH, and GHX, wherein X can be any amino acid, such as in:

KGH, RGH, HGH, GGH, AGH, VGH, LGH, IGH, MGH, FGH, WGH, PGH, SGH, TGH, CGH, YGH, NGH, QGH, DGH, EGH, and KGH; or

GKH, GRH, GHH, GGH, GAH, GVH, GLH, GIH, GMH, GFH, GWH, GPH, GSH, GTH, GCH, GYH, GNH, GQH, GDH, GEH, and GLH; or

GHK, GHR, GHH, GHG, GHA, GHV, GHL, GHI, GHM, GHF, GHW, GHP, GHS, GHT, GHC, GHY, GHN, GHQ, GHD, GHE, and GHL;

XGF, GXF, and GFX, wherein X can be any amino acid, such as in:

KGF, RGF, HGF, GGF, AGF, VGF, LGF, IGF, MGF, FGF, WGF, PGF, SGF, TGF, CGF, YGF, NGF, QGF, DGF, EGF, and KGF; or

GKF, GRF, GHF, GGF, GAF, GVF, GLF, GIF, GMF, GFF, GWF, GPF, GSF, GTF, GCF, GYF, GNF, GQF, GDF, GEF, and GLF; or

GFK, GFR, GFH, GFG, GFA, GFV, GFL, GFI, GFM, GFF, GFW, GFP, GFS, GFT, GFC, GFY, GFN, GFQ, GFD, GFE, and GFL;

XRL, RXL, and RLX, wherein X can be any amino acid, such as in:

KRL, RRL, HRL, GRL, ARL, VRL, LRL, IRL, MRL, FRL, WRL, PRL, SRL, TRL, CRL, YRL, NRL, QRLRL, DRL, ERL, and KRL; or

GKF, GRF, GHF, GGF, GAF, GVF, GLF, GIF, GMF, GFF, GWF, GPF, GSF, GTF, GCF, GYF, GNF, GQF, GDF, GEF, and GLF; or

RLK, RLR, RLH, RLG, RLA, RLV, RLL, RLI, RLM, RLF, RLW, RLP, RLS, RLT, RLC, RLY, RLN, RLQ, RLD, RLE, and RLL;

XHP, HXP, and HPX, wherein X can be any amino acid, such as in:

KHP, RHP, HHP, GHP, AHP, VHP, LHP, IHP, MHP, FHP, WHP, PHP, SHP, THP, CHP, YHP, NHP, QHP, DHP, EHP, and KHP; or

HKP, HRP, HHP, HGP, HAF, HVF, HLF, HIF, HMF, HFF, HWF, HPF, HSF, HTF, HCF, HYP, HNF, HQF, HDF, HEF, and HLP; or

HPK, HPR, HPH, HPG, HPA, HPV, HPL, HPI, HPM, HPF, HPW, HPP, HPS, HPT, HPC, HPY, HPN, HPQ, HPD, HPE, and HPL;

Particularly preferred examples are: CRPYC (SEQ ID NO: 83), KCRPYC (SEQ ID NO: 84), KHCRPYC (SEQ ID NO: 85), RCRPYC (SEQ ID NO: 86), HCRPYC (SEQ ID NO: 87), CPRYC (SEQ ID NO: 88), KCPRYC (SEQ ID NO: 89), RCPRYC (SEQ ID NO: 90), HCPRYC (SEQ ID NO: 91), CPYRC (SEQ ID NO: 92), KCPYRC (SEQ ID NO: 93), RCPYRC (SEQ ID NO: 94), HCPYRC (SEQ ID NO: 95), CKPYC (SEQ ID NO: 96), KCKPYC (SEQ ID NO: 97), RCKPYC (SEQ ID NO: 98), HCKPYC (SEQ ID NO: 99), CPKYC (SEQ ID NO: 100), KCPKYC (SEQ ID NO: 101), RCPKYC (SEQ ID NO: 102), HCPKYC (SEQ ID NO: 103), CPYKC (SEQ ID NO: 104), KCPYKC (SEQ ID NO: 105), RCPYKC (SEQ ID NO: 106), and HCPYKC (SEQ ID NO: 107);

(e) Zₘ-[CST]-Xₙ-C- or Zₘ-C-Xₙ-[CST]- as defined in aspect 2, wherein n is 4, thereby creating an internal X¹X²X³X⁴ (SEQ ID NO: 111) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In a specific embodiment, at least one of X¹, X², X³ or X⁴ in said motif is a basic amino acid selected from: H, K, or R, or a non-natural basic amino acid as defined herein.

Specific examples are: LAVL (SEQ ID NO: 108), TVQA (SEQ ID NO: 109) or GAVH (SEQ ID NO: 110) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 112 to 122); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein;

(f) Zₘ-[CST]-Xₙ-C- or Zₘ-C-Xₙ-[CST]- as defined in aspect 2, wherein n is 5, thereby creating an internal X¹X²X³X⁴X⁵ (SEQ ID NO: 125) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid selected from: H, K, or R, or a non-natural basic amino acid as defined herein.

Specific examples are: PAFPL (SEQ ID NO: 123) or DQGGE (SEQ ID NO: 124) and their variants such as: X¹AFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 126 to 135), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein;

(g) Zₘ-[CST]-Xₙ-C- or Zₘ-C-Xₙ-[CST]- as defined in aspect 2, wherein n is 6, thereby creating an internal X¹X²X³X⁴X⁵X⁶ (SEQ ID NO: 137) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H, and wherein B is any amino acid. Preferred are motifs wherein m is 1 or 2. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T.

In a specific embodiment, at least one of X¹, X², X³ X⁴, X⁵ or X⁶ in said motif is a basic amino acid selected from: H, K, or R, or a non-natural basic amino acid as defined herein.

Specific examples are: DIADKY (SEQ ID NO: 136) or variants thereof such as: X¹IADKY, DX²ADKY, DIX³DKY, DIAX⁴KY, DIADX⁵Y, or DIADKX⁶ (corresponding to SEQ ID NO: 138 to 143), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein; or

(h) Zₘ-[CST]-Xₙ-C- or Zₘ-C-Xₙ-[CST]- as defined in aspect 2, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group (SEQ ID NO: 144 to 163).

In preferred embodiments of such a motif, n is 2, and m is 1 or 2, wherein the internal X¹X², each individually, can be any amino acid selected from the group consisting of: G, A, V, L, I, M, F, W, P, S, T, C, Y, N, Q, D, E, K, R, and H, or non-natural amino acids.

Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).

Aspect 19. The pharmaceutical kit according to any one of aspects 1 to 18, wherein said epitope is derived from the Myelin-oligodendrocyte glycoprotein (MOG) antigen amino acid sequence. More preferably said epitope is selected from the group comprising amino acid residues: 40-60, 41-55, 43-57, 44-58, 45-59, and 35-55 of the mature MOG amino acid sequence defined by SEQ ID NO: 208:

GQFRVIGPRHPIRALVGDEVELPCRISPGKNATGMEVGWYRPPFSRVVH
LYRNGKDQDGDQAPEYRGRTELLKDAIGEGKVTLRIRNVRFSDEGGFTC
FFRDHSYQEEAAMELKVEDPFYWVSPGVLVLLAVLPVLLLQITVGLIFL
CLQYRLRGKLRAEIENLHRTFDPHFLRVPCWKITLFVIVPVLGPLVALI
ICYNWLHRRLAGQFLEELRNPF,

Such as those selected from the group comprising:

(SEQ ID NO: 200)
YRPPFSRVVHLYRNGKDQDGD
(SEQ ID NO: 201)
RPPFSRVVHLYRNGK
(SEQ ID NO: 202)
PFSRVVHLYRNGKDQ
(SEQ ID NO: 203)
FSRVVHLYRNGKDQD
(SEQ ID NO: 204)
SRVVHLYRNGKDQDG
(SEQ ID NO: 164)
FLRVPCWKI
(SEQ ID NO: 165)
FLRVPSWKI
(SEQ ID NO: 170)
WHLYRNGK
(mouse SEQ ID NO: 205)
MEVGWYRSPFSRVVHLYRNGK,
(human SEQ ID NO: 206)
MEVGWYRPPFSRVVHLYRNGK,
(mouse SEQ ID NO: 169)
YRSPFSRVV,
and
(human SEQ ID NO: 168)
YRPPFSRVV,

or combinations thereof.

Aspect 20. The pharmaceutical kit according to any one of aspects 1 to 18 wherein the epitope in said immunogenic or tolerogenic peptide is derived from the myelin proteolipid protein (also called proteolipid protein (PLP) or lipohilin) antigen amino acid sequence.

More preferably, with reference to patent application WO2014111841, said epitope is selected from the group comprising amino acid residues: 36-61, 179-206, 207-234, 39-57, 180-198, 208-222, 39-53, 42-56, 43-57, 180-194, 181-195, 182-196, 183-197, 184-198, 208-222, 36-61, 179-206, and 207-234 of the PLP amino acid sequence defined by SEQ ID NO: 207 (UniProtKB—P60201 (MYPR_HUMAN)):

MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF

Such as those selected from the group comprising:

PLP 36-61:
(SEQ ID NO: 209)
HEALTGTEKLIETYFSKNYQDYEYLI;
PLP 179-206:
(SEQ ID NO: 210)
TWTTCQSIAFPSKTSASIGSLCADARMY;
PLP 207-234:
(SEQ ID NO: 211)
GVLPWNAFPGKVCGSNLLSICKTAEFQM
PLP 39-57:
(SEQ ID NO: 212)
LTGTEKLIETYFSKNYQDY
PLP 180-198:
(SEQ ID NO: 213)
WTTCQSIAFPSKTSASIGS
PLP 208-222:
(SEQ ID NO: 214)
VLPWNAFPGKVCGSN
PLP 39-53:
(SEQ ID NO: 215)
LTGTEKLIETYFSKN
PLP 42-56:
(SEQ ID NO: 216)
TEKLIETYFSKNYQD
PLP 43-57:
(SEQ ID NO: 217)
EKLIETYFSKNYQDY
PLP 180-194:
(SEQ ID NO: 218)
WTTCQSIAFPSKTSA
PLP 181-195:
(SEQ ID NO: 219)
TTCQSIAFPSKTSAS
PLP 182-196:
(SEQ ID NO: 220)
TCQSIAFPSKTSASI
PLP183-197:
(SEQ ID NO: 221)
CQSIAFPSKTSASIG
PLP 184-198:
(SEQ ID NO: 222)
QSIAFPSKTSASIGS
PLP 208-222:
(SEQ ID NO: 223)
VLPWNAFPGKVCGSN
PLP 36-61:
(SEQ ID NO: 224)
HEALTGTEKLIETYFSKNYQDYEYLI
PLP 179-206:
(SEQ ID NO: 225)
TWTTCQSIAFPSKTSASIGSLCADARMY
and
PLP 207-234:
(SEQ ID NO: 226)
GVLPWNAFPGKVCGSNLLSICKTAEFQM

or combinations thereof.

Aspect 21. The pharmaceutical kit according to any one of aspects 1 to 18, wherein the epitope in said immunogenic or tolerogenic peptide is derived from the myelin basic protein (MBP) antigen amino acid sequence. More preferably said MBP epitope is selected from the group comprising the following sequences:

(SEQ ID NO: 227)
PRHRDTGILDSIGRF
(SEQ ID NO: 228)
ENPVVHFFKNIVTPRTP
(SEQ ID NO: 229)
RASDYKSAHKGFKGV
(SEQ ID NO: 230)
GFKGVDAQGTLSKIF
(SEQ ID NO: 231)
LGGRDSRSGSPMARR
(SEQ ID NO: 232)
TQDENPVVHFFKNIVTPRTP
(SEQ ID NO: 233)
TQDENPVVHFFKNIV
(SEQ ID NO: 234)
QDENPVVHFFKNIVT
(SEQ ID NO: 235)
DENPVVHFFKNIVTP
(SEQ ID NO: 236)
ENPVVHFFKNIVTPR
(SEQ ID NO: 237)
NPVVHFFKNIVTPRT
(SEQ ID NO: 238)
PVVHFFKNIVTPRTP
(SEQ ID NO: 239)
ASDYKSAHKGFKGVDAQGTLSKIFKLGG
(SEQ ID NO: 240)
ASDYKSAHKGFKGVD
(SEQ ID NO: 241)
SDYKSAHKGFKGVDA
(SEQ ID NO: 242)
DYKSAHKGFKGVDAQ
(SEQ ID NO: 243)
YKSAHKGFKGVDAQG
(SEQ ID NO: 244)
KSAHKGFKGVDAQGT
(SEQ ID NO: 245)
SAHKGFKGVDAQGTL
(SEQ ID NO: 246)
AHKGFKGVDAQGTLS
(SEQ ID NO: 247)
HKGFKGVDAQGTLSK
(SEQ ID NO: 248)
KGFKGVDAQGTLSKI
(SEQ ID NO: 249)
GFKGVDAQGTLSKIF
(SEQ ID NO: 250)
FKGVDAQGTLSKIFK
(SEQ ID NO: 251)
KGVDAQGTLSKIFKL
(SEQ ID NO: 252)
GVDAQGTLSKIFKLG
(SEQ ID NO: 253)
VDAQGTLSKIFKLGG,
and
(SEQ ID NO: 254)
LSRFSWGAEGQRPG,

or combinations thereof,
or any one or more of the fragments defined by amino acid residues 30-44, 80-94, 83-99, 81-95, 82-96, 83-97, 84-98, 110-124, 130-144, 131-158, 131-145, 140-148, 142-152, 132-146, 134-148, 135-149, 136-150, 137-151, 138-152, 139-153, 140-154 and 133-147 of the MBP amino acid sequence defined by SEQ ID NO: 255 (UniProtKB—P02686-5 (MBP_HUMAN)):

MASQKRPSQRHGSKYLATASTMDHARHGFLPRHRDTGILDSIGRFFGGDR
GAPKRGSGKDSHHPARTAHYGSLPQKSHGRTQDENPVVHFFKNIVTPRTP
PPSQGKGRGLSLSRFSWGAEGQRPGFGYGGRASDYKSAHKGFKGVDAQGT
LSKIFKLGGRDSRSGSPMARR.

In a preferred embodiment, said MBP epitope is selected from the group comprising the sequences defined in SEQ ID NO: 227 to 230 or is a combination of any 2, 3 or 4 thereof.

Referring to the publication of Streeter et al., 2015, Neurol Neuroimmunol Neuroinflamm. 2015 June; 2(3): e93, reporting on the clinical trial with this particular cocktail of all 4 peptides defined in SEQ ID NO: 227 to 230, called ATX-MS-1467, is particularly preferred.

Aspect 22. The pharmaceutical kit according to any one of aspects 2 to 21, wherein said immunogenic peptide has an oxidoreductase motif which comprises the sequence CC, KCC, RCC, CRC, CKC, KCRC (SEQ ID NO: 65), KCKC (SEQ ID NO: 63), RCKC (SEQ ID NO: 171), RCRC (SEQ ID NO: 67), CPYC (SEQ ID NO: 172), HCPYC (SEQ ID NO: 71), KCPYC (SEQ ID NO: 72), RCPYC (SEQ ID NO: 73), CRPYC (SEQ ID NO: 83), CPRYC (SEQ ID NO: 88), CPYRC (SEQ ID NO: 92), CKPYC (SEQ ID NO: 96), CPKYC (SEQ ID NO: 100), CPYKC (SEQ ID NO: 104), RCRPYC (SEQ ID NO: 86), RCPRYC (SEQ ID NO: 90), RCPYRC (SEQ ID NO: 94), RCKPYC (SEQ ID NO: 98), RCPKYC (SEQ ID NO: 102), RCPYKC (SEQ ID NO: 106), KCRPYC (SEQ ID NO: 84), KCPRYC (SEQ ID NO: 89), KCPYRC (SEQ ID NO: 93), KCKPYC (SEQ ID NO: 97), KCPKYC (SEQ ID NO: 101), or KCPYKC (SEQ ID NO: 105).

Aspect 23. The pharmaceutical kit according to any one of aspects 2 to 22, wherein said immunogenic peptide has a linker with sequence VRY between the oxidoreductase motif and the T-cell epitope.

Aspect 24. The pharmaceutical kit according to any one of aspects 2 to 23, wherein said immunogenic peptide comprises or consists essentially of the amino sequence:

HCPYCVRYFLRVPSWKITLF (SEQ ID NO: 174),

HCPYCVRYFLRVPCWKITLF (SEQ ID NO: 175),

KHCPYCVRYFLRVPSWKITLFKK (SEQ ID NO: 176), or

KHCPYCVRYFLRVPCWKITLFKK (SEQ ID NO: 177). Aspect 25. The pharmaceutical kit according to any one of aspects 1 to 24, for use in treatment of, ameliorating the symptoms of, and/or preventing of a fumarate-related disease or disorder, preferably selected from the group consisting of: auto-immune disorders, demyelinating disorders, transplant rejection or cancer. Preferred examples of such diseases and disorders are: Multiple Sclerosis (MS), Neuromyelitis Optica (NMO), psoriasis, Rheumatoid Arthritis (RA), asthma, atopic dermatitis, scleroderma, ulcerative colitis, cancer, and transplant rejection.

Aspect 26. The pharmaceutical kit for use, according to aspect 25, wherein said fumarate compound and said immunogenic or tolerogenic peptide are administered simultaneously, sequentially and/or separately.

Aspect 26. The pharmaceutical kit for use, according to aspect 25 or 26, wherein said immunogenic or tolerogenic peptide is administered before said fumarate composition, preferably at least 12 hours before, such as at least 24 hours before, more preferably at least 1 to 20 or at least 1 to 10 days before treatment with said fumarate compound is started. In certain embodiments, the administration (injection) of the immunogenic or tolerogenic peptide is repeated once, twice, three times, four times, five or six times, each with an interval of between 1 to 20 days, or between 1 to 10 days.

Aspect 27. the pharmaceutical kit for use, according to aspect 25 or 26, wherein the following chronological treatment scheme is applied:
1) Fumarate compound treatment comprising a once, or twice daily administration of the fumarate compound for a period of at least 4 weeks, such as at least 1, 2, or 3 months to up to 4, 5, or 6 months;
2) commencement of immunogenic or tolerogenic peptide treatment (injection) for at least 1, 2, 3 or 4 times, each with an interval of between 1 to 10 days, such as of between 5 to 9 days, e.g. of about 7 days, optionally while the fumarate treatment as in 1) is being maintained during the peptide treatment;
Optionally a step 3) wherein after steps 1 and 2) are competed, the Fumarate compound treatment as in 1) is maintained when needed;
Optionally a step 4) can be implemented with one or more immunogenic or tolerogenic peptide boost administrations, done 1, 2, or 3 months after the last immunogenic peptide administration, each boost again given with an interval of between 1 to 20 days or between 1 to 10 days, such as of between 5 to 9 days, e.g. of about 7 days.

The preferred dosage regimens of the fumarate compound and immunogenic or tolerogenic peptide are defined elsewhere in the application in more detail.

A particularly preferred treatment regimen for the DMF fumarate compound is 120 mg twice a day for the first seven days, after which it is increased to 240 mg twice a day.

A particularly but non-limiting dosage regimen of the immunogenic or tolerogenic peptide as defined herein is between 50 and 1500 μg, preferably between 450 and 1500 μg. Dosage regimen can comprise the administration in a single dose or in 2, 3, 4, 5, 6 or more doses, either simultaneously or consecutively.

Aspect 28. The pharmaceutical kit for use, according to any one of aspects 25 to 27, wherein the treatment with the fumarate is done daily or twice per day, and/or wherein the treatment with the immunogenic or tolerogenic peptide is done 1 to 6 times, such as 1 to 4 times, preferably every 5 to 9 days, such as about every 7 days.

Aspect 29. The pharmaceutical kit for use, according to any one of aspects 25 to 28, wherein said fumarate composition is administrated before, during and optionally after the administration of the immunogenic or tolerogenic peptide.

Aspect 30. The pharmaceutical kit for use, according to any one of aspects 25 to 29, wherein said fumarate compound is administered orally once or twice per day, and/or wherein said immunogenic or tolerogenic peptide is administered through subcutaneous injection.

Aspect 30. A method of treatment of, ameliorating the symptoms of, and/or preventing of a fumarate-related disease or disorder in a patient in need thereof comprising the step of administering an effective amount of the dose units of the pharmaceutical kit according to any one of aspects 1 to 24.

Aspect 31. The method according to aspect 30, wherein said fumarate-related disease or disorder is an auto-immune disorder, a demyelinating disorder, transplant rejection or cancer. Preferred examples of such diseases and disorders are: Multiple Sclerosis (MS), psoriasis, Neuromyelitis optica (NMO), Rheumatoid Arthritis (RA), polyarthritis, asthma, atopic dermatitis, scleroderma, ulcerative colitis, juveline diabetes, thyreoiditis, Grave's disease, Systemic Lupus Erythromatosis (SLE), Sjögren syndrome, anemia perniciosa, chronic active hepatitis, transplant rejection and cancer.

Aspect 32. The method according to aspect 30 or 31, wherein said fumarate compound and said immunogenic or tolerogenic peptide are administered simultaneously, sequentially and/or separately.

Aspect 33. The method according to any one of aspects 30 to 32, wherein said immunogenic or tolerogenic peptide is administered before said fumarate composition, preferably at least 12 hours before, such as at least 24 hours before, more preferably at least 1 to 20 days, such as at least 1 to 10 days before treatment with said fumarate compound is started, such as 5 to 9 days, e.g. about 7 days before said treatment with said fumarate compound is started. In certain embodiments, the administration (injection) of the immunogenic or tolerogenic peptide is repeated once, twice, three times, four times, five or six times, each with an interval of between 1 to 20 days, such as of between 1 and 10 days, such as of between 5 to 9 days, e.g. of about 7 days.

Alternatively, the following chronological treatment scheme is applied:
1) Fumarate compound treatment comprising a once, or twice daily administration of the fumarate compound for a period of at least 4 weeks, such as at least 1, 2, or 3 months to up to 4, 5, or 6 months;
2) commencement of immunogenic or tolerogenic peptide treatment (injection) for at least 1, 2, 3 or 4 times, each with an interval of between 1 to 20 days, such as of between 1 and 10 days, such as of between 5 to 9 days, e.g. of about 7 days, optionally while the fumarate treatment as in 1) is being maintained during the peptide treatment;
Optionally a step 3) wherein after steps 1 and 2) are competed, the Fumarate compound treatment as in 1) is maintained when needed;
Optionally a step 4) can be implemented with one or more immunogenic peptide or tolerogenic boost administrations, done 1, 2, or 3 months after the last immunogenic or tolerogenic peptide administration, each boost again given with an interval of between 1 to 20 days, such as of between 1 and 10 days, such as of between 5 to 9 days, e.g. of about 7 days.

The preferred dosage regimens of the fumarate compound and immunogenic or tolerogenic peptide are defined elsewhere in the application in more detail. A particularly preferred treatment regimen for the DMF fumarate compound is 120 mg twice a day for the first seven days, after which it is increased to 240 mg twice a day.

Aspect 34. The method according to any one of aspects 30 to 33, wherein the treatment with the fumarate is done daily or twice per day, and/or wherein the treatment with the immunogenic or tolerogenic peptide is done 1 to 6 times, such as 1 to 4 times, preferably every 5 to 9 days, such as about every 7 days.

Aspect 35. The method according to any one of aspects 30 to 34, wherein said fumarate composition is administrated before, during and optionally after the administration of the immunogenic or tolerogenic peptide.

Aspect 40. The method according to any one of aspects 30 to 35, wherein said fumarate compound is administered orally once or twice per day, and/or wherein said immunogenic or tolerogenic peptide is administered through subcutaneous injection.

Aspect 41. Preferably, said tolerogenic peptide is administered through mucosal delivery such as through nasal, oral, buccal, pulmonary, ocular, vaginal, or rectal delivery; or through, intradermal administration, transdermal administration or subcutaneously injection. Preferably, said tolerogenic peptide should be administered in soluble form in the absence of adjuvant.

Aspect 42. Preferably, said immunogenic peptide is administered through intradermal administration, transdermal administration or subcutaneously injection. Preferably, said immunogenic peptide should be administered in soluble form in the presence of adjuvant.

Aspect 43. A nucleic acid encoding the immunogenic or tolerogenic peptide according to any one of the aspects or examples disclosed herein, preferably selected from isolated desoxyribonucleic acid (DNA), plasmid DNA (pDNA), coding DNA (cDNA), ribonucleic acid (RNA), messenger RNA (mRNA) or modified versions thereof. In some embodiments, said nucleic acid can be part of an expression cassette, optionally incorporated in a (viral) vector or plasmid that can be used for gene-therapy or can be present in the form of encapsulated or naked DNA or RNA to be administered according to techniques known in the pharmaceutical and gene therapeutic field.

Aspect 44. In any one of the aspects disclosed herein relating to methods of treatment or medical uses of either the tolerogenic or immunogenic peptide, said peptides can also be administered as a nucleic acid encoding said respective peptide in accordance with aspect 43.

Aspect 45. In any one of the aspects described herein,
when said fumarate-related disease or disorder is MS, said antigen is preferably recognized in the context of HLA-DRB1*15:01, HLA-DRB1*03:01, HLA-DRB1*04:01, HLA-DRB1*07:01, HLA DRB5*0101, or DQ6 type of HLA. More preferred are patients having a HLA-DRB1* type 15:01;
when said fumarate-related disease or disorder is NMO, said antigen is preferably recognized in the context of HLA-DRB1*03:01 or HLA-DPB1*05:01 (for Asia); or
when said fumarate-related disease or disorder is RA, said antigen is preferably recognized in the context of HLA-DRB1*01:01, 04:01 or 04:04.

### BRIEF DESCRIPTION OF THE FIGURES

FIG. 1: represents blinded evaluation of clinical EAE scoring (0-5) performed daily from day 7 to day 28. Mice were prophylactically immunized or not with IMCY-0189, then injected with MOG₃₅₋₅₅ to induce EAE at day 0, and were treated or not with BG-12 (see table 1 for details). The mean clinical score was determined each day for each group of mice.

FIG. 2: represents AUC calculated from EAE scores displayed in FIG. 1 for each group of mice. Significant differences are referred as follows: *p<0.05, **p<0.01, ***p<0.001, ****p<0.0001.

FIG. 3: represents MMS calculated from EAE scores displayed in FIG. 1 for each group of mice. Significant differences are referred as follows: *p<0.05, **p<0.01, ***p<0.001, ****p<0.0001.

FIG. 4: represents inflammation levels for each group of mice presented in table 1. Inflammatory foci of approximately 20 cells were counted in each H&E stained section. When inflammatory infiltrates consisted of more than 20 cells, an estimate was made of how many foci of 20 cells were present. Significant differences are referred as follows: *p<0.05, **p<0.01, ***p<0.001, ****p<0.0001.

FIG. 5: represents demyelination levels for each group of mice presented in table 1. Demyelination was scored in each anti-MBP (using immunohistochemistry) stained section. The demyelination score represents an estimate of demyelinated area for each section. Significant differences are referred as follows: *p<0.05, **p<0.01, ***p<0.001, ****p<0.0001.

FIG. 6: represents plasma neurofilaments levels for each group of mice presented in table 1. Neurofilament light (NF-L) protein levels were quantified at Quanterix™ through the NF-light Simoa® assay advantage kit. Significant differences are referred as follows: *p<0.05, **p<0.01, ***p<0.001, ****p<0.0001.

FIG. 7: represents blinded evaluation of clinical EAE scoring (0-5) performed daily from day 7 to day 28. Mice were injected with MOG₃₅₋₅₅ to induce EAE at day 0, and were left untreated or therapeutically treated with IMCY-0189 or MOG₃₅₋₅₅, in combination or not with BG-12 (see table 3 for details). The mean clinical score was determined each day for each group of mice.

FIG. 8: represents AUC calculated from EAE scores displayed in FIG. 7 for each group of mice. Significant differences are referred as follows: *p<0.05, **p<0.01, ***p<0.001, ****p<0.0001.

FIG. 9: represents MMS calculated from EAE scores displayed in FIG. 7 for each group of mice. Significant differences are referred as follows: *p<0.05, **p<0.01, ***p<0.001, ****p<0.0001.

FIG. 10: represents blinded evaluation of clinical EAE scoring (0-5) performed daily from day 7 to day 28. Mice were injected with MOG₃₅₋₅₅ to induce EAE at day 0, and were left untreated or therapeutically treated with IMCY-0189, IMCY-0453 or IMCY-0455, in combination or not with BG-12 (see table 4 for details). The mean clinical score was determined each day for each group of mice.

FIG. 11: represents AUC calculated from EAE scores displayed in FIG. 10 for each group of mice. Significant differences are referred as follows: *p<0.05, **p<0.01, ***p<0.001, ****p<0.0001.

FIG. 12: represents MMS calculated from EAE scores displayed in FIG. 10 for each group of mice. Significant differences are referred as follows: *p<0.05, **p<0.01, ***p<0.001, ****p<0.0001.

FIG. 13: represents serum neurofilaments levels for each group of mice presented in table 4. Neurofilament light (NF-L) protein levels were quantified at Quanterix™ through the NF-light Simoa® assay advantage kit. Significant differences are referred as follows: *p<0.05, **p<0.01, ***p<0.001, ****p<0.0001.

FIG. 14: represents blinded evaluation of clinical EAE scoring (0-5) performed daily from day 7 to day 28. Mice were injected with MOG₃₅₋₅₅ to induce EAE at day 0, and were left untreated or therapeutically treated with IMCY-0189 or P4, in combination or not with BG-12 (see table 5 for details). The mean clinical score was determined each day for each group of mice.

FIG. 15: represents AUC calculated from EAE scores displayed in FIG. 14 for each group of mice. Significant differences are referred as follows: *p<0.05, **p<0.01, ***p<0.001, ****p<0.0001.

FIG. 16: represents MMS calculated from EAE scores displayed in FIG. 14 for each group of mice. Significant differences are referred as follows: *p<0.05, **p<0.01, ***p<0.001, ****p<0.0001.

FIG. 17: represents serum neurofilaments levels for each group of mice presented in table 5. Neurofilament light (NF-L) protein levels were quantified at Quanterix™ through the NF-light Simoa® assay advantage kit. Significant differences are referred as follows: *p<0.05, **p<0.01, ***p<0.001, ****p<0.0001.

### DETAILED DESCRIPTION OF THE INVENTION

As used herein, the singular forms “a”, “an”, and “the” include both singular and plural referents unless the context clearly dictates otherwise. By way of example, an immunogenic peptide refers to one or more than one immunogenic peptide.

The terms “comprising”, “comprises” and “comprised of” as used herein are synonymous with “including”, “includes” or “containing”, “contains”, and are inclusive or open-ended and do not exclude additional, non-recited members, elements or method steps. Said terms also encompass the embodiments “consisting essentially of” and “consisting of”.

As used herein, the term “for use” as used in “preparation for use in treatment of a disease” shall disclose also the corresponding method of treatment and the corresponding use of a preparation for the manufacture of a medicament for the treatment of a disease.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The term “any” when used in relation to aspects, claims or embodiments as used herein refers to any single one (i.e. anyone) as well as to all combinations of said aspects, claims or embodiments referred to.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

The terms “pharmaceutical kit”, “pharmaceutical combination”, “pharmaceutical composition” or “pharmaceutical kit-of-parts”, as used herein can be used interchangeably, define especially a “kit-of-parts” in the sense that the different active ingredients, i.e. the fumarate compound and the immunogenic or tolerogenic peptide as defined herein can be dosed independently, i.e. are present in said kit in different unit doses or dosage forms. Said separate dosage forms can be administered simultaneously and/or at different time points, such as chronologically staggered, that is at different time points and with equal or different time intervals for any part of the kit-of-parts. The ratio of the total amounts of the combination partners to be administered in the combined preparation can be varied. The combination partners can be administered by the same route or by different routes.

As used herein, the term “fumarate composition” or “fumarate agent” refers to a composition according to general formula (I)

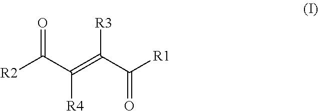

wherein R¹ and R² each independently are selected from the groups consisting of: OH, O⁻, and optionally substituted (C₁₋₁₀)alkoxy, preferably optionally substituted (C₁₋₆)alkoxy, or optionally substituted (C₁₋₃)alkoxy,
wherein R³ and R⁴ each independently are selected from the groups consisting of: H or deuterium,
wherein each group independently can be optionally substituted to form a prodrug of MMF as outlined herein elsewhere. Note that in some embodiments, DMF is in fact a prodrug of MMF, which is the active ingredient.

In preferred embodiments said (C₁₋₁₀)alkoxy can be chosen from: (C₁₋₅)alkoxy, (C₁₋₄)alkoxy, (C₁₋₃)alkoxy, ethoxy, ethoxy, (C₂₋₃)alkoxy, (C₂₋₄)alkoxy, (C₂₋₅) alkoxy, and (C₁₋₆)alkoxy.

In preferred embodiments, said fumarate compound is a monoalkyl fumarate, a dialkyl fumarate or a combination thereof.

In non-limiting illustrative embodiments, the fumarate compound of formula (I) are: dimethyl fumarate—DMF (R¹ is OCH₃ and R² is OCH₃— Formula (II)) or monomethyl fumarate—MMF (R¹ is OCH₃ and R² is O— or OH—Formula (III)), or is in the form of a prodrug of monomethyl fumarate.

The term “substituted” or “optionally substituted” as used herein, refers to a group in which one or more hydrogen atoms can each independently be replaced with the same or different substituent group(s). In certain embodiments, each substituent group is independently halogen, —OH, —CN, —CF₃, ═O, —NO₂, benzyl, —C(O)NH₂, —R″, —OR″, —C(O)R″, —COOR″, —S(O)₂R″ or —NR₂″ wherein each R″ is independently hydrogen or (C₁₋₆)alkyl, cycloalkyl, cycloalkylalkyl, alkenyl, alkynyl, arylalkyl, aryl, alkanediyl, heteroalkyl, heterocycloalkyl, heteroaryl or heteroalylalkyl, heterocycloalkylalkyl. In certain embodiments, each substituent group is independently halogen, —OH, —CN, —CF₃, —NO₂, benzyl, —R″, —OR″, or

—NR₂″ wherein each R″ is independently hydrogen or (C₁₋₄)alkyl. In certain embodiments, each substituent group is independently halogen, —OH, —CN, —CF₃, ═O, —NO₂, benzyl, —C(O)NR₂″, —R″, —OR″, —C(O)R″, —COOR″, or —NR₂″ wherein each R″ is independently hydrogen or (C₁₋₄)alkyl. In certain embodiments, each substituent group is independently —OH, (Cl₄)alkyl, and NH₂.

A “prodrug of monomethyl fumarate” is a compound of formula (I) wherein R1, R2, R3, or R4, each independently is optionally substituted by a chemical group which is capable of being removed in vivo, i.e. after administration to the patient. A prodrug hence is a compound that can be metabolized into monomethyl fumarate in vivo and results in the active form, i.e. monomethyl fumarate.

Preferred examples of said fumarate compounds are prodrugs of monoalkylfumarate or more specifically monomethylfumarate, i.e. compounds that can be metabolized into monomethyl fumarate in vivo, such as those compounds of Formula (I) wherein R¹ is C₁-C₃ alkoxy such as methoxy, ethoxy or propoxy and wherein R² is C₁-C₃ alkoxy such as methoxy, ethoxy or propoxy which is optionally substituted.

Further preferred examples are those wherein R¹ is methoxy and R² is optionally substituted methoxy or optionally substituted ethoxy.

Non-limiting examples of such pro-drugs are the ones disclosed in any one of the following patent applications or patents: WO2016081355, WO2015105757A1, WO2014/096425, WO2014031901, WO2014152494, WO2013/119677, U.S. Pat. No. 8,669,281 B1, and US2014/0179779. Preferred examples of prodrugs of MMF are DMF (formula (II), diroximel fumarate (formula (IV) or tepilamide fumarate (formula (V).

In further preferred embodiments, fumarate compound of formula (I) is in the form of a pharmaceutically acceptable salt of mono- or dimethyl fumarate, such as an acid addition salt. Acid addition salts are formed by mixing a solution of a fumarate with a solution of a pharmaceutically acceptable non-toxic acid such as hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, gluconate, glucaronate, saccharate, formate, benzoate, glutamate, methanesulfonate, ethanesulfonate, benzenesulfonate, p-toluenesulfonate, and pamoate. Acceptable base salts include aluminum, calcium, lithium, magnesium, potassium, sodium, zinc, and diethanolamine salts. base addition salts of the fumarates provided herein include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Others are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995). In some preferred embodiments, the pharmaceutically acceptable salt is a salt of a metal (M) cation, wherein M can be an alkali, alkaline earth, or transition metal such as Li, Na, K, Ca, Zn, Sr, Mg, Fe, or Mn. In a preferred embodiment said salt is Ca-MMF or Ca-DMF.

The fumarate compound as defined herein can be formulated into a composition.

The term “stereoisomer” as used herein refers to one stereoisomer of a fumarate compound of formula (I) that is substantially free of other stereoisomers of that fumarate. For example, a “stereomerically pure” fumarate having one chiral center will be substantially free of the opposite enantiomer of the fumarate. A “stereomerically pure” fumarate having two chiral centers will be substantially free of the other diastereomers of the fumarate. A typical “stereomerically pure” fumarate compound comprises greater than about 80% by weight of one stereoisomer of the fumarate and less than about 20% by weight of other stereoisomers of the fumarate, greater than about 90% by weight of one stereoisomer of the fumarate and less than about 10% by weight of the other stereoisomers of the fumarate, greater than about 95% by weight of one stereoisomer of the fumarate and less than about 5% by weight of the other stereoisomers of the fumarate, or greater than about 97% by weight of one stereoisomer of the fumarate and less than about 3% by weight of the other stereoisomers of the fumarate. The fumarate compound can have chiral centers and can occur as racemates, individual enantiomers or diastereomers, and mixtures thereof. All such isomeric forms are included within the embodiments disclosed herein, including mixtures thereof. The use of stereomerically pure forms of such fumarates, as well as the use of mixtures of those forms, are encompassed by the embodiments disclosed herein. For example, mixtures comprising equal or unequal amounts of the enantiomers of a particular fumarate may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley Interscience, New York, 1981); Wilen, S. H., et al, Tetrahedron 33:2725 (1977); Eliel, E. L., Stereochemistry of Carbon Compounds (McGraw Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

In further embodiments, said fumarate compound of formula (I) is deuterated, i.e. wherein one or more of the hydrogen atoms in the formula is deuterated, such as in the form of a deuterated alkyl group, such as a deuterated methyl group that contains at least one deuterium atom. Examples of deuterated methyl include:

—CDH₂, —CD₂H, and —CD₃. Examples of deuterated ethyl include: —CHDCH₃, —CD₂CH₃, —CHDCDH₂, —CHDCD₂H, CHDCD₃, —CD₂CDH₂, —CD₂CD₂H, and —CD₂CD₃.

The term “alkoxy” as used herein is an alkyl (carbon and hydrogen chain) group singularly bonded to oxygen.

The term “alkyl,” as used herein, refers to a fully saturated branched or unbranched hydrocarbon moiety. In one embodiment, the alkyl comprises 1 to 10 carbon atoms, 1 to 16 carbon atoms, 1 to 10 carbon atoms, 1 to 6 carbon atoms, or 1 to 3 carbon atoms. Representative examples of alkyl groups include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

The term “alkanediyl,” as used herein refers to linear or branched alkyl chains with, for example 1 to 6 carbon atoms. Representative examples of aklanediyl groups include, but are not limited to —CH₂—, —(CH₂)₂, —CH(CH₃)—, —(CH₂)₃—, —CH₂CH(CH₃)—, —CH(CH₃)CH₂—, —CH(C₂H)—, —C(CH₃)₂—, —(CH₂)₄—, —(CH₂)₂CH(CH₃)—, —CH₂CH(CH₃)CH₂—, —CH(CH₃)(CH₂)₂—, —CH(C₂H₅)CH₂—, —CH₂CH(C₂H₅)—, —C(CH₃)₂CH₂—, —CH₂C(CH₃)₂—, —CH(CH₃)CH(CH₃), —CH(C₃H)—, —(CH₂)₅, —(CH₂)₃CH(CH₃), —(CH₂)₂CH(CH₃)CH₂—, —CH₂CHCH₃(CH₂)₂—, —CH₂C(CH₃)₂CH₂—, —(CH₂)₂C(CH₃)₂—, —(CH₂)₆—, —(CH₂)₄CH(CH₃)—, —(CH₂)₃CH(CH₃)CH₂—, —CH₂CHCH₃(CH₂)₃—, —(CH₂)₃C(CH₃)₂—, and —(CH₂)₂C(CH₃)₂CH₂—.

The term “alkenyl,” as used herein, refers to a monovalent straight or branched chain hydrocarbon having from two to six carbons and at least one carbon-carbon double bond. Representative examples of alkenyl groups include, but are not limited to, —CH═CH₂, —CH═CH—CH₃, —CH₂—CH═CH—CH₃, or —CH(CH₃)—CH═CH—CH₃.

The term “alkynyl,” as used herein, refers to a monovalent straight or branched chain hydrocarbon having from two to six carbons and at least one carbon-carbon triple bond. Representative examples of alkynyl groups include, but are not limited to, 2-propynyl, 3-butynyl, 2-butynyl, 4-pentynyl, 3-pentynyl.

The term “aryl,” as used herein, refers to monocyclic, bicyclic or tricyclic aromatic hydrocarbon groups having, for example, from 5 to 14 carbon atoms in the ring portion.

In one embodiment, the aryl refers to monocyclic and bicyclic aromatic hydrocarbon groups having from 6 to 10 carbon atoms. Representative examples of aryl groups include, but are not limited to, phenyl, naphthyl, fluorenyl, and anthracenyl.

The term “arylalkyl,” as used herein, refers to an acyclic alkyl group in which one of the hydrogen atoms bonded to a carbon atom, typically a terminal or sp³ carbon atom, is replaced with an aryl group. Representative examples of arylalkyl groups include, but are not limited to, benzyl, 2-phenylethan-1-yl, naphthylmethyl, 2-naphthylethan-1-yl, naphthobenzyl, or 2-naphthophenylethan-1-yl. In certain embodiments, an arylalkyl group is C₇₋₃₀arylalkyl, e.g., the alkyl moiety of the arylalkyl group is C₁₋₁₀ and the aryl moiety is C₆₋₂₀. In certain embodiments, an arylalkyl group is C₆₋₁₈ arylalkyl, e.g., the alkyl moiety of the arylalkyl group is C₁₋₈ and the aryl moiety is C₆₋₁₀. In certain embodiments, the arylalkyl group is C₇₋₁₂ arylalkyl.

The term “cycloalkyl,” as used herein, refers to a saturated or partially unsaturated cyclic alkyl group. Representative examples of cycloalkyl groups include, but are not limited to, cyclopropane, cyclobutane, cyclopentane, or cyclohexane. In one embodiment, a cycloalkyl group is C₃₁₅ cycloalkyl, C₃₋₁₂ cycloalkyl, or C₃₋₃ cycloalkyl.

The term “cycloalkylalkyl,” as used herein, refers to an acyclic alkyl group in which one of the hydrogen atoms bonded to a carbon atom, typically a terminal or sp³ carbon atom, is replaced with a cycloalkyl group. In certain embodiments, cycloalkylalkyl group is C₄₋₃₀ cycloalkylalkyl, and, for example, the alkyl moiety of the cycloalkylalkyl group is C₁₋₁₀ and the cycloalkyl moiety is C₃₋₂₀. In another embodiment, a cycloalkylalkyl group is C₃₋₂₀ cycloalkylalkyl, and, for example, the alkyl moiety of the cycloalkylalkyl group is C₁₋₃ and the cycloalkyl moiety is C₃₋₁₂. In a particular embodiment, a cycloalkylalkyl group is C₄₋₁₂ cycloalkylalkyl.

The term “halogen,” as used herein, refers to fluoro, chloro, bromo, or iodo.

The term “heteroalkyl,” as used herein, by itself or as part of another substituent refers to an alkyl group in which one or more of the carbon atoms (and certain associated hydrogen atoms) are independently replaced with heteroatomic groups. Examples of heteroatomic groups include, but are not limited to, —O—, —S—, —O—O—, —S—S—, —O—S—, —NR′, ═N—N═, —N═N—, —N═N—NR′—, —PR′—, —P(O)2-, —POR′—, —O—P(O)₂—, —SO—, —SO2-, and —Sn(R′)2-, where each R′ is independently hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₃ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. In one embodiment, a C₁₋₆ heteroalkyl, means, for example, a C₁₋₆ alkyl group in which at least one of the carbon atoms (and certain associated hydrogen atoms) is replaced with a heteroatom. In a particular embodiment, a C₁₋₆ heteroalkyl, for example, includes groups having five carbon atoms and one heteroatom, groups having four carbon atoms and two heteroatoms, etc. In one embodiment, each R′ is independently hydrogen or C₁₋₃ alkyl. In another embodiment, a heteroatomic group is —O—, —S—, —NH—, —N(CH₃)—, or —SO₂—. In a specific embodiment, the heteroatomic group is —O—.

The term “heteroaryl,” as used herein, refers to, for example, a 5-14 membered monocyclic-, bicyclic-, or tricyclic-ring system, having 1 to 10 heteroatoms independently selected from N, O, or S, wherein N and S can be optionally oxidized to various oxidation states, and wherein at least one ring in the ring system is aromatic. In one embodiment, the heteroaryl is monocyclic and has 5 or 6 ring members. Representative examples of monocyclic heteroaryl groups include, but are not limited to, pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is bicyclic and has from 8 to 10 ring members. Representative examples of bicyclic heteroaryl groups include indolyl, benzofuranyl, quinolyl, isoquinolyl indazolyl, indolinyl, isoindolyl, indolizinyl, benzamidazolyl, quinolinyl, 5,6,7,8-tetrahydroquinoline, and 6,7-dihydro-5H-pyrrolo[3,2-d]pyrimidine. In another embodiment, the heteroaryl is bicyclic and has from 8 to 10 ring members.

The term “heteroarylalkyl,” as used herein, refers to an acyclic alkyl group in which one of the hydrogen atoms bonded to a carbon atom, typically a terminal or sp3 carbon atom, is replaced with a heteroaryl group. In certain embodiments, a heteroarylalkyl group is C₇₋₁₂ heteroarylalkyl, and, for example, the alkyl moiety of the heteroarylalkyl group is C₁₋₂ and the heteroaryl moiety is C₆₋₁₀.

The term “heterocycle” as used herein, refers to any ring structure (saturated or partially unsaturated) which contains at least one ring heteroatom (e.g., N, O or S). Examples of heterocycles include, but are not limited to, morpholine, pyrrolidine, tetrahydrothiophene, piperidine, piperazine and tetrahydrofuran.

The term “heterocycloalkyl,” as used herein, refers to a saturated or unsaturated cyclic alkyl group in which one or more carbon atoms (and certain associated hydrogen atoms) are independently replaced with one or more heteroatoms; or to a parent aromatic ring system in which one or more carbon atoms (and certain associated hydrogen atoms) are independently replaced with one or more heteroatoms such that the ring system no longer contains at least one aromatic ring. Representative examples of heteroatoms to replace the carbon atom(s) include, but are not limited to, N, P, O, S, and Si. Representative examples of heterocycloalkyl groups include, but are not limited to, epoxides, azirines, thiuranes, imidazolidine, morpholine, piperazine, piperidine, pyrazolidine, pyrrolidine, and quinuclidine. In one embodiment, a heterocycloalkyl group is C₅₋₁₀ heterocycloalkyl, C₅₋₈ heterocycloalkyl. In a specific embodiment, a heterocycloalkyl group is C₅₋₆ heterocycloalkyl.

The term “heterocycloalkylalkyl,” as used herein, refers to an acyclic alkyl group in which one of the hydrogen atoms bonded to a carbon atom, typically a terminal or sp³ carbon atom, is replaced with a heterocycloalkyl group. In certain embodiments, a heterocycloalkylalkyl group is C₇₋₁₂ heterocycloalkylalkyl, and, for example, the alkyl moiety of the heterocycloalkylalkyl group is C₁₋₂ and the heterocycloalkyl moiety is C₅₋₁₀.

In one embodiment, the fumarate compound as defined herein for use in the kits and methods of the invention is present in the form of a fumarate pharmaceutical composition or dosage form comprising a therapeutically effective amount of the fumarate compound as defined herein and a pharmaceutically acceptable carrier or pharmaceutically acceptable excipient.

In a specific embodiment, the fumarate pharmaceutical composition or dosage form comprises a fumarate compound selected from the group comprising: a dialkyl fumarate, a monoalkyl fumarate, a combination of a dialkyl fumarate and a monoalkyl fumarate, a prodrug of monoalkyl fumarate, a deuterated form of any of the foregoing, or a clathrate, solvate, tautomer, or stereoisomer of any of the foregoing, or a combination of any of the foregoing.

In a specific embodiment, the fumarate pharmaceutical composition or dosage form consists essentially of DMF and/or MMF.

The fumarate pharmaceutical composition or dosage form can be administered in many ways. U.S. Pat. Nos. 6,509,376 and 6,436,992 for example disclose some possible formulations containing DMF and/or MMF. As to route of administration, the compositions can be administered orally, intranasally, transdermally, subcutaneously, intradermally, vaginally, intraorally, intraocularly, intramuscularly, buccally, rectally, transmucosally, or via inhalation, or intravenous administration. In some embodiments DMF or MMF is administered orally.

In a specific embodiment, the fumarate pharmaceutical composition or dosage form can be an oral dosage form, e.g., a solid oral dosage form e.g., micro-pellets, micro-tablets, a capsule (such as a soft or hard gelatine capsule), a granule, or a tablet. In a specific embodiment, the fumarate pharmaceutical composition or dosage form is in a form of micro-pellets or micro-tablets, a capsule, or capsule containing micro-tablets or micro-pellets.

Optionally, the micro-tablets or micro-pellets or capsules are enterically coated. In a specific embodiment, the fumarate pharmaceutical composition or dosage form is in the form of enterically coated tablets or microtablets (optionally contained in a capsule), which, once the enteric coating is dissolved in the gastro-intestinal tract, act as immediate release dosage forms.

In another specific embodiment, the fumarate pharmaceutical composition or dosage form is a controlled, or sustained, release composition, optionally enterically coated. Such formulations can be prepared by various technologies by a skilled person in the art. For example, the formulation can contain the therapeutic compound, a rate-controlling polymer (i.e., a material controlling the rate at which the therapeutic compound is released from the dosage form) and optionally other excipients. Some examples of rate-controlling polymers are hydroxy alkyl cellulose, hydroxypropyl alkyl cellulose (e.g., hydroxypropyl methyl cellulose, hydroxypropyl ethyl cellulose, hydroxypropyl isopropyl cellulose, hydroxypropyl butyl cellulose and hydroxypropyl hexyl cellulose), poly(ethylene)oxide, alkyl cellulose (e.g., ethyl cellulose and methyl cellulose), carboxymethyl cellulose, hydrophilic cellulose derivatives, and polyethylene glycol, compositions described in WO 2006/037342.

The fumarate compound or preparation as defined herein may be combined with pharmaceutically acceptable excipients or carrier, and optionally sustained-release matrices, such as biodegradable polymers, to form a pharmaceutical formulation. In this pharmaceutical formulation, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical formulations or dosage forms contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The fumarate pharmaceutical compositions or preparations described herein are manufactured in a manner which is itself known, for example, by means of conventional mixing, granulating, dragee-making, dissolving, or lyophilizing processes. Thus, fumarate pharmaceutical preparations for oral use may be obtained by combining the fumarates with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearne or calcium stearne, and or polyethylene glycol.

Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments may be added to the tablets or dragee coatings, for example, for identification or in order to characterize combinations of active compound doses.

In one embodiment, the fumarate pharmaceutical preparation or dosage form described herein comprises a capsule containing the pharmaceutical composition described herein in the form of an enteric-coated microtablet. The coating of the microtablet may be composed of different layers. The first layer may be a methyacrylic acid—methyl methacrylate copolymer/isopropyl solution which isolates the tablet cores from potential hydrolysis from the next applied water suspensions.

The enteric coating of the tablet may then be conferred by an aqueous methacrylic acid-ethyl acrylate copolymer suspension.

The number of excipients that can be included in a composition is not limited.

Examples of fillers or binders include, but are not limited to, ammonium alginate, calcium carbonate, calcium phosphate, calcium sulfate, cellulose, cellulose acetate, compressible sugar, confectioner's sugar, dextrates, dextrin, dextrose, erythritol, ethylcellulose, fructose, glyceryl palmitostearate, hydrogenated vegetable oil type I, isomalt, kaolin, lactitol, lactose, mannitol, magnesium carbonate, magnesium oxide, maltodextrin, maltose, mannitol, medium chain triglycerides, microcrystalline cellulose, polydextrose, polymethacrylates, simethicone, sodium alginate, sodium chloride, sorbitol, starch, sucrose, sugar spheres, sulfobutylether beta-cyclodextrin, talc, tragacanth, trehalsoe, polysorbate 80, and xylitol. In one embodiment, the filler is microcrystalline cellulose. The microcrystalline cellulose can be, for example, PROSOLV SMCC® 50, PROSOLV SMCC® 90, PROSOLV SMCC® HD90, PROSOLV SMCC® 90 LM, and any combination thereof.

Examples of disintegrants include, but are not limited to, hydroxypropyl starch, alginic acid, calcium alginate, carboxymethylcellulose calcium, carboxymethylcellulose sodium, powdered cellulose, chitosan, colloidal silicon dioxide, croscarmellose sodium, crospovidone, docusate sodium, guar gum, hydroxypropyl cellulose, low substituted hydroxypropyl cellulose, magnesium aluminum silicate, methylcellulose, microcrystalline cellulose, polacrilin potassium, povidone, sodium alginate, sodium starch glycolate, starch, and pregelatinized starch. In one embodiment, the disintegrant is croscarmellose sodium.

Examples of glidants include, but are not limited to, calcium phosphate, calcium silicate, powdered cellulose, magnesium silicate, magnesium triplicate, silicon dioxide, talcum and colloidal silica, and colloidal silica anhydrous. In one embodiment, the glidant is colloidal silica anhydrous, talc, or a combination thereof.

Examples of lubricants include, but are not limited to, canola oil, hydroxyethyl cellulose, lauric acid, leucine, mineral oil, poloxamers, polyvinyl alcohol, talc, octyldodecanol, sodium hyaluronate, sterilizable maize starch, triethanolamine, calcium stearate, magnesium stearne, glycerin monostearate, glyceryl behenate, glyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, potassium benzoate, sodium benzoate, sodium chloride, sodium lauryl sulfate, stearic acid, talc, and zinc stearne. In one embodiment, the lubricant is magnesium stearne.

The fumarate pharmaceutical composition or dosage form suitable for the methods described above include without limitation those formulated for once daily (QD) dosing or multiple dosing per day (e.g., twice a day (BID) dosing, or three times a day (TTD) dosing). In some embodiments, the pharmaceutical composition is formulated for QD dosing, wherein the therapeutically effective amount of a fumarate compound as defined herein (e.g., DMF or MMF) is included in one-unit dosage form or provided in a kit containing multiple unit dosage forms. In some embodiments, the pharmaceutical composition is formulated for a BID or TID dosing, wherein the therapeutically effective amount of a fumarate compound as defined herein (e.g., DMF or MMF) is divided, for example, equally, for dosing two or three times daily.

The therapeutically effective amount of a fumarate compound as defined herein (e.g., DMF or MMF) may be any therapeutically effective dose. In some embodiments, the neurological disorder is multiple sclerosis, wherein the therapeutically effective amount of a fumarate compound as defined herein (e.g., DMF or MMF) is an amount that is effective in treating or preventing multiple sclerosis, for example, in a subject who is characterized as a non-responder to interferon beta treatment.

In some embodiments, the fumarate agent is DMF, and suitable (i.e. therapeutically effective) doses of DMF may be any dose from 20 mg to 1 g of DMF. In some embodiments, the DMF in the pharmaceutical composition is about 60 mg, about 80 mg, about 100 mg, about 120 mg, about 160 mg, about 200 mg, about 240 mg, about 320 mg, about 360 mg, about 400 mg, about 480 mg, about 600 mg, about 720 mg, about 800 mg, about 900 mg, about 1000 mg of DMF, or any ranges thereof. In some embodiments, the therapeutically effective amount of DMF is about 480 mg or about 720 mg per day. In some embodiments, the about 480 mg DMF is provided in two-unit dosage forms, each comprises about 240 mg DMF and is dosed to a subject about 6 hours to about 14 hours apart in a day. In some embodiments, the about 720 mg DMF is provided in three-unit dosage forms, each comprises about 240 mg DMF and is dosed to a subject about 4 hours to about 8 hours apart in a day.

In one embodiment, the administering is of 240 mg twice daily of dimethyl fumarate.

In one embodiment, the administering is of 120 mg dimethyl fumarate twice daily for 7 days, followed by 240 mg dimethyl fumarate twice daily as a maintenance dose.

In one embodiment, the administering is of not greater than 720 mg daily total fumarates.

In one embodiment, the administering is of not greater than 480 mg daily total fumarates.

In one embodiment, the pharmaceutical composition consists essentially of dimethyl fumarate, and the administering is of not greater than 720 mg daily dimethyl fumarate.

In one embodiment, the pharmaceutical composition consists essentially of dimethyl fumarate, and the administering is of not greater than 480 mg daily dimethyl fumarate.

In a preferred embodiment, said fumarate compound is dimethyl-fumarate or a derivative thereof such as the drug commercialized under the trade name TECFIDERA™. (Formula II):

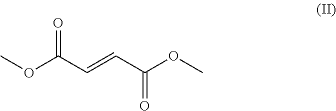

In another embodiment, said fumarate compound is in the form of a pharmaceutical composition commercialized under the tradename FUMADERM™ comprising as active ingredients: dimethyl fumarate, calcium salt of ethyl hydrogen fumarate, magnesium salt of ethyl hydrogen fumarate, and zinc salt of ethyl hydrogen fumarate.

The term “peptide” as used herein refers to a molecule comprising an amino acid sequence of between 9 and 50 amino acids in case of using an NKT cell epitope of minimally 7 amino acids or, between 9 and 50, preferably between 11 and 50 amino acids when using an MHC class II T cell epitope with minimal length of 7, 8, or 9 amino acids, connected by peptide bonds, but which can comprise non-amino acid structures. Peptides according to the invention can contain any of the conventional 20 amino acids or modified versions thereof, or can contain non-naturally occurring amino acids incorporated by chemical peptide synthesis or by chemical or enzymatic modification.

The peptides of the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains, etc.

Chemical peptide synthesis methods are well described and peptides can be ordered from companies such as Applied Biosystems and other companies.

In any aspect of the present invention and unless indicated otherwise, the term “peptide” can mean an immunogenic peptide or a tolerogenic peptide as defined herein.

The immunogenic or tolerogenic peptides of the present invention can vary substantially in length. The length of the peptides can vary from 9 or 11 amino acids, i.e. consisting of an epitope of 7, 8 or 9 amino acids, adjacent thereto the modified oxidoreductase oxidoreductase motif of from 2 to about 11 amino acids, to up to 20, 25, 30, 40 or 50 amino acids. For example, a peptide may comprise an endosomal targeting sequence of 40 amino acids, a flanking sequence of about 2 amino acids, an oxidoreductase motif as described herein of from 2 to about 11 amino acids, a linker of 4 to 7 amino acids and a T cell epitope peptide of 7, 8 or 9 amino acids minimal length.

Accordingly, in particular embodiments, the complete peptide consists of between 9 amino acids to up 10, 11, 12, 13, 14, 15, 16, 17, 18, 19, 20, 25, 30, 40, 50, 75 or 100 amino acids. More particularly, where the reducing compound is a modified oxidoreductase motif as described herein, the length of the (artificial or natural) sequence comprising the epitope and modified oxidoreductase motif optionally connected by a linker (referred to herein as ‘epitope-modified oxidoreductase motif’ sequence), without the endosomal targeting sequence, is critical. The ‘epitope-modified oxidoreductase motif’ more particularly has a length of 9, 10, 11, 12, 13, 14, 15, 16, 17, 18 or 19 amino acids. Such peptides of 9, 10, 11, 12, 13 or 14 to 19 amino acids can optionally be coupled to an endosomal targeting signal of which the size is less critical.

In a specific embodiment, the peptides of the invention have a length of between 9 and 30 or of between 11 and 30 amino acids.

According to one embodiment, the immunogenic or tolerogenic peptide of the invention comprises an NKT cell epitope and has a length of between 9 and 30 amino acids.

According to another embodiment, the immunogenic or tolerogenic peptide of the invention comprises a MCH class II T cell epitope and has a length of between 11 and 30 amino acids.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)—OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0)

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives are indicated by listing the acceptable amino acids for a given position, between square brackets (‘[ ]’). For example: [CST] stands for an amino acid selected from Cys, Ser or Thr. Amino acids which are excluded as alternatives are indicated by listing them between curly brackets (‘{ }’). For example: {AM} stands for any amino acid except Ala and Met. The different elements in a motif are optionally separated from each other by a hyphen (-). In the context of the motifs disclosed in this specification, the disclosed general oxidoreductase motifs are typically accompanied by a hyphen not forming a connection with a different element outside the motif. These ‘open’ hyphens indicate the position of the physical connection of the motif with another portion of the immunogenic peptide such as a linker sequence or an epitope sequence. For example, a motif of the form “Zₘ-C-Xₙ-[CST]-” indicates that the [CST] is the amino acid connected to the other portion of the immunogenic peptide, and Z is a terminal amino acid of the immunogenic peptide. Preferred physical connections are peptide bonds. Repetition of an identical element within a motif can be indicated by placing behind that element a numerical value or a numerical range between parentheses. For example In this respect, “Xₙ” refers to n-times “X”. X(2) corresponds to X-X or XX; X(2, 5) corresponds to 2, 3, 4 or 5 X amino acids, A(3) corresponds to A-A-A or AAA. To distinguish between the amino acids, those outside the oxidoreductase motif can be called external amino acids, those within the oxidoreductase motif are called internal amino acids. Unless stated otherwise X represents any amino acid, particularly an L-amino acid, more particularly one of the 20 naturally occurring L-amino acids.

The term “antigen” as used herein refers to a structure of a macromolecule, typically protein (with or without polysaccharides) or made of proteic composition comprising one or more hapten(s) and comprising T or NKT cell epitopes. The term “antigenic protein” as used herein refers to a protein comprising one or more T or NKT cell epitopes. An “auto-antigen” or “auto-antigenic protein” as used herein refers to a human or animal protein or fragment thereof present in the body, which elicits an immune response within the same human or animal body.

The term “epitope” refers to one or several portions (which may define a conformational epitope) of an antigenic protein which is/are specifically recognized and bound by an antibody or a portion thereof (Fab′, Fab2′, etc.) or a receptor presented at the cell surface of a B, T or NKT cell, and which is able, by said binding, to induce an immune response.

The term “T cell epitope” in the context of the present invention refers to a dominant, sub-dominant or minor T cell epitope, i.e. a part of an antigenic protein that is specifically recognized and bound by a receptor at the cell surface of a T lymphocyte. Whether an epitope is dominant, sub-dominant or minor depends on the immune reaction elicited against the epitope. Dominance depends on the frequency at which such epitopes are recognized by T cells and able to activate them, among all the possible T cell epitopes of a protein. In the context of the immunogenic peptides defined herein, the T cell epitope can be an epitope recognized by MHC class II molecules, which consists of a sequence of +/−9 amino acids which fit in the groove of the MHC II molecule. Within a peptide sequence representing a T cell epitope, the amino acids in the epitope are numbered P1 to P9, amino acids N-terminal of the epitope are numbered P−1, P−2 and so on, amino acids C terminal of the epitope are numbered P+1, P+2 and so on. Peptides recognized by MHC class II molecules and not by MHC class I molecules are referred to as MHC class II restricted T cell epitopes. Alternatively, in the context of the immunogenic peptides defined herein, the T cell epitope can be an epitope recognized by CD1d molecules, which consists of a sequence of +/−7 amino acids which bind the CD1d molecule. Within a peptide sequence representing a T cell epitope, the amino acids in the epitope are numbered P1 to P7, amino acids N-terminal of the epitope are numbered P−1, P−2 and so on, amino acids C terminal of the epitope are numbered P+1, P+2 and so on. Peptides recognized by CD1d molecules and not by MHC molecules are referred to as CD1d or NKT-restricted T cell epitopes.

In light of the tolerogenic peptides as defined herein, the T cell epitope can be either an MHC, such as MHCI or MHCII, or an NKT epitope and can be longer.

The identification and selection of a T-cell epitope from antigenic proteins is known to a person skilled in the art.

This finding provides a rule-based method for selection of tolerogenic T cell epitopes which obviates the need to examine the tolerogenic capacity of a peptide in vivo. This is particularly advantageous in the development of strategies to treat or prevent diseases for which no animal models are available. Even for diseases which have an animal model, the selection method should make the development of tolerance-inducing compositions simpler and safer, because it provides a mechanism whereby the tolerance induction capacity of a peptide can be tested on human T cells (recognising antigen in conjunction with human MHC molecules) in vitro, prior to their use in vivo.

Typical methods for selecting an immunogenic or tolerogenic peptide comprises the step of selecting a peptide which is capable of binding to an MHC class I or class II molecule as reported in e.g. WO0216410A2. In a preferred embodiment, the peptide is capable of binding to an MHC class II molecule.

A number of methods are known in the art for screening for immunogenic tolerogenic peptides which are capable of acting as T cell epitopes for a given antigen. Commonly, therefore, the method will be used to select a tolerogenic peptide from a plurality of peptides each comprising a T cell epitope.

The term “tolerogenic” means capable of inducing tolerance, i.e. substantial failure to respond to an antigen. Tolerance to self or auto-antigens is an essential feature of the immune system and disturbances therein can lead to autoimmune diseases. Tolerance in general is generated in the thymus (central tolerance), where self-reactive immature T lymphocytes undergo apoptosis. However, there is also a mechanism by which tolerance is acquired by mature self-reactive T lymphocytes in the peripheral tissues (peripheral tolerance). In the context of the present application a tolerogenic peptide does not comprise an oxidoreductase motif as defined herein.

The mechanism of central and peripheral tolerance is reported e.g. in Anderton et al (1999) (Immunological Reviews 169: 123-137). Tolerance may result from or be characterised by the induction of anergy in at least a portion of CD4+ T cells. In order to activate a T cell, a peptide must associate with a “professional” APC capable of delivering two signals to T cells. The first signal (signal 1) is delivered by the MHC-peptide complex on the cell surface of the APC and is received by the T cell via the TCR. The second signal (signal 2) is delivered by costimulatory molecules on the surface of the APC, such as CD80 and CD86, and received by CD28 on the surface of the T cell. It is thought that when a T cell receives signal 1 in the absence of signal 2, it is not activated and, in fact, becomes anergic. Anergic T cells are refractory to subsequent antigenic challenge, and may be capable of suppressing other immune responses. Anergic T cells are thought to be involved in mediating T cell tolerance. It has been shown that, when tolerance is induced by peptide inhalation, the capacity of antigen-specific CD4+ T cells to proliferate is reduced. Also, the production of IL-2, IFN-γ and IL-4 production by these cells is down-regulated, but production of IL-10 is increased. Neutralisation of IL-10 in mice in a state of peptide-induced tolerance has been shown to restore completely susceptibility to disease. It has been proposed that a population of regulatory cells persist in the tolerant state which produce IL-10 and mediate immune regulation (Burkhart et al (1999) Int. Immunol. 11: 1625-1634). The induction of tolerance can therefore be monitored by various techniques including: (a) reduced susceptibility to contract the disease for which the peptide is a target epitope in vivo; (b) the induction of anergy in CD4+ T cells (which can be detected by subsequent challenge with antigen in vitro); (c) changes in the CD4+ T cell population, including (i) reduction in proliferation; (ii) down-regulation in the production of IL-2, IFN-y and IL-4; and (iii) increase in the production of IL-10.

Tolerogenic peptides as used herein encompass all antigen-derived peptides and T-cell epitopes that induce tolerance (anergy) towards the antigen they are derived from.

Epitope selection comprises the step of selecting a peptide which is capable of binding to an MHC class I or II protein. Epitopes can be immunodominant, i.e. hotspots in the antigen that are presented by APCs more often than others. Immunodominant determinant regions are likely to be good tolerogens and hence in a preferred embodiment, the tolerogenic peptide, or epitope of the present invention is based on an immunodominant epitope. However, during auto-immune disease development, epitope spreading may occur towards sub-dominant determinants (Lehmann et al (1992) Nature 358: 155-157). Presentation of sub-dominant epitopes may hence also be important in triggering autoimmunity and the tolerogenic peptide, or epitope of the present invention may, therefore be based on a subdominant epitope. Last but not least, the tolerogenic peptide, or epitope of the present invention may be a cryptic epitope, i.e. an epitope which can stimulate a T cell response when administered as a peptide but which fails to produce a response to the antigen when administered as a whole.

Naturally processed epitopes may be identified by mass spectrophotometric analysis of peptides eluted from antigen-loaded APC, i.e. APC that have either been encouraged to take up antigen, or have been forced to produce the protein intracellularly by transformation with the appropriate gene. Typically APC are incubated with protein either in solution or suitably targeted to the APC cell surface. After incubation at 37° C. the cells are lysed in detergent and the class II protein purified by, for example affinity chromatography. Treatment of the purified MHC with a suitable chemical medium (for example, acid conditions) results in the elution of peptides from the MHC. This pool of peptides is separated and the profile compared with peptide from control APC treated in the same way. The peaks unique to the protein-expressing cells are analysed (for example by mass spectrometry) and the peptide fragments identified. This procedure usually generates information about the range of peptides (usually found in “nested sets”) generated from a particular antigen by antigen processing.

Another method for identifying epitopes is to screen a synthetic library of peptides which overlap and span the length of the antigen in an in vitro assay. For example, peptides which are 15 amino acids in length and which overlap by 5 or 10 amino acids may be used. The peptides are tested in an antigen presentation system which comprises antigen presenting cells and T cells. For example, the antigen presentation system may be a murine splenocyte preparation, a preparation of human cells from tonsil or PBMC T cell activation may be measured via T cell proliferation (for example using 3H-thymidine incorporation) or cytokine production. Activation of THI-type CD4+ T cells can, for example be detected via IFNy production which may be detected by standard techniques, such as an ELISPOT assay. Such overlapping peptide studies usually indicate the area of the antigen in which an epitope is located. The minimal epitope for a particular T cell can then be assessed by measuring the response to truncated peptides. For example if a response is obtained to the peptide comprising residues 1-15 in the overlapping library, sets which are truncated at both ends (i. e. 1-14, 1-13, 1-12 etc. and 2-15, 3-15, 4-15 etc.) can be used to identify the minimal epitope.

The identification of immunodominant regions of an antigen using in vitro assays (especially those using T cell lines) is predicted to present a skewed pattern of peptide reactivity by the present inventors. A kinetic response assay in which the proliferation of PBMC from patients and healthy individuals is measured against an overlapping peptide library can be used. This assay is based on the finding that, although T cells form normal individuals and patients respond in a similar fashion to purified protein antigen, they respond in a different way to peptides based on the sequence of the antigen. T cells from auto-immune patients respond with greater magnitude and more rapid kinetics to peptide auto-antigens when compared with normal healthy donors. This enables screening for and identification of the epitope to which the particular patient responds at a particular time.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from an individual having a fumarate-related disease or disorder with a peptide/epitope derived from the auto-antigen involved in said disease or disorder and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class II molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that MHC class II molecule. Peptides to be assessed for class II binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxy-terminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types.

The term “immunogenic” in the context of the present invention means capable of inducing so called cytolytic CD4+ T cells, i.e. T cells with apoptotic properties against APCs as described in details in WO2009101207 and Carlier et al. (2012) Plos one 7, 10 e45366.

The term “Immunogenic peptide” refers to a peptide comprising an oxidoreductase motif as defined herein.

The term “oxidoreductase motif”, “thiol-oxidoreductase motif”, “thioreductase motif”, “thioredox motif” or “redox motif” are used here as synonymous terms and refers to a motif of general sequence thioreductase sequence motif C-Xₙ-[CST]- (SEQ ID NO: 26 to 30) or [CST]-Xₙ-C- (SEQ ID NO: 1 to 5), with n being an integer from 0 to 6. Such peptide motives exert reducing activity for disulfide bonds on proteins (such as enzymes) through redox active cysteines within conserved active domain consensus sequences: C-Xₙ-[CST]- or [CST]-Xₙ-C-, such as for example in C-XX-C, C-XX-S, C-XX-T, S-XX-C, T-XX-C(SEQ ID NO: 187 to 191) (Fomenko et al. (2003) Biochemistry 42, 1 1214-1 1225), in which X stands for any amino acid, in which C stands for cysteine, S for serine, T for threonine and X for any amino acid except tyrosine, phenylalanine or tryptophan. In a further embodiment thereto, said oxidoreductase motif is positioned N-terminally of the T-cell epitope. Alternatively, the immunogenic peptides may contain an oxidoreductase motif in the form of the following general amino acid formula: Zₘ-[CST]-Xₙ-C- (SEQ ID NO: 1 to 25) or Zₘ-C-Xₙ-[CST]- (SEQ ID NO: 26 to 50) as defined herein elsewhere, wherein n is an integer chosen from 0 to 6, wherein m is an integer selected from 0 to 3, wherein X is any amino acid, wherein Z is any amino acid, in which C stands for cysteine, S for serine, T for threonine.

The terms “cysteine”, “C”, “serine”, “S”, and “threonine”, “T”, when used in the light of the amino acid residues present in the oxidoreductase motifs disclosed herein respectively refer to naturally occurring cysteine, serine or threonine amino acids. Unless explicitly stated differently, said terms hence exclude chemically modified cysteines, serines and threonines such as those modified so as to carry an acetyl, methyl, ethyl or propionyl group, either on the N-terminal amide of the amino acid residue of the motif or on the C-terminal carboxy group.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from an individual having a fumarate-related disease or disorder with a peptide/epitope derived from the auto-antigen involved in said disease or disorder and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class II molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that MHC class II molecule. Peptides to be assessed for class II binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxy-terminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEES Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAs DQB1, HLA-DRA, and HLADRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2). Class I MHC molecules are expressed on virtually all nucleated cells. Since the HLA system is inherited in a Mendelian manner, HLA series of genes, or haplotypes, can be distinguished in subjects of a given population.

In the global MS patient population, about 50% to 60% have HLA-DRB1* type 15:01. Further, over 75% of the MS patient population has a HLA-DRB1*15:01, HLA-DRB1*03:01, HLA-DRB1*04:01, or HLA-DRB1*07:01 type of HLA. A preferred HLA type of a patient in view of the current invention is therefore selected from the group consisting of: DRB1*15:01, HLA-DRB1*03:01, HLA-DRB1*04:01, and HLA-DRB1*07:01. More preferred are patients having a HLA-DRB1* type 15:01. Further preferred are RRMS diagnosed MS patients having an HLA type selected from the group consisting of: DRB1*15:01, HLA-DRB1*03:01, HLA-DRB1*04:01, and HLA-DRB1*07:01. Further preferred are RRMS diagnosed MS patients having an HLA type HLA-DRB1*15:01.

Preferred HLA haplotypes in NMO are HLA-DRB1*03:01 and HLA-DPB1*05:01 (for Asia).

Preferred HLA haplotypes in RA are HLA-DRB1*01:01, 04:01 and 04:04.

In humans, the MHC is divided into three regions: class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2). Class I MHC molecules are expressed on virtually all nucleated cells. Peptide fragments presented in the context of class I MHC molecules are recognized by CD8+T lymphocytes (cytotoxic T lymphocytes or CTLs). CD8+T lymphocytes frequently mature into cytotoxic effectors which can lyse cells bearing the stimulating antigen. Class II MHC molecules are expressed primarily on activated lymphocytes and antigen-presenting cells. CD4+T lymphocytes (helper T lymphocytes or Th) are activated with recognition of a unique peptide fragment presented by a class II MHC molecule, usually found on an antigen presenting cell like a macrophage, a B cell or dendritic cell. CD4+T lymphocytes proliferate and secrete cytokines such as IL-2, IFN-gamma and IL-4 that support antibody mediated and cell mediated responses.

Functional HLAs are characterized by a deep binding groove to which endogenous as well as foreign, potentially antigenic peptides bind. The groove is further characterized by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 7, 8, 9 or 10 residues. However, it has been demonstrated that peptides of up to 12 amino acid residues are also capable of binding HLA class 1. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 7 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class 1. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P−2 and so on, amino acids C-terminal of the epitope are numbered P+1, P+2 and so on.

The term “NKT cell epitope” refers to a part of an antigenic protein that is specifically recognized and bound by a receptor at the cell surface of an NKT cell. In particular, a NKT cell peptide epitope is an epitope bound by CD1d molecules, with motif [FWHY]-XX-[ILMV]-XX-[FWTHY] [SEQ ID NO: 51] or a more restrictive form thereof, such as [FW]-XX-[ILMV]-XX-[FW] [SEQ ID NO: 52]. In this motif, F stands for phenylalanine, W for tryptophan, H for histidine, Y for tyrosine, I for isoleucine, L for leucine, M for methionine, V for valine, and X for any amino acid. [FWHY] indicates that either F, W, H or Y can occupy the first anchoring residue (P1), that the P4 position can be occupied by either I, L, M or V, and that P7 can be occupied by F, W, H or Y. It should be clear for the one skilled in the art that various combinations of these amino acid residues are possible.

The term “NKT cells” refers to cells of the innate immune system characterized by the fact that they carry receptors such as NK1.1 and NKG2D, and recognize peptide epitopes presented by the CD1d molecule. In the context of the present invention, NKT cells can belong to either the type 1 (invariant) or the type 2 subset, or to any of the less characterized NKT cells with more polymorphic T cell receptors than type 1 or type 2 NKT cells.

The “CD1d molecule” refers to a non-MHC derived molecule, expressed at the surface of various APCs, made of 3 alpha chains and an anti-parallel set of beta chains arranged into a deep hydrophobic groove opened on both sides and capable of presenting lipids, glycolipids or hydrophobic peptides to NKT cells.

The present invention provides methods for generating antigen-specific cytolytic CD4+ T cells either in vivo or in vitro and, independently thereof, methods to discriminate cytolytic CD4+ T cells from other cell populations such as Foxp3+ Tregs based on characteristic expression data.

The immunogenic peptides as defined herein comprise an oxidoreductase motif of the following general amino acid sequence: Zₘ-[CST]-Xₙ-C- (SEQ ID NO: 1 to 25) or Zₘ-C-Xₙ-[CST]- (SEQ ID NO: 26 to 50) as defined in aspect 2, is selected from the following amino acid motifs:

(a) Zₘ-[CST]-Xₙ-C- or Zₘ-C-Xₙ-[CST]- as defined in aspect 2, wherein n is 0, and wherein

wherein m is an integer selected from 0 to 3,
wherein Z is any amino acid, preferably a basic amino acid selected from: H, K, R, and a non-natural basic amino acid as defined herein, such as L-ornithine, preferably K or R, most preferably K.

In preferred embodiments of motif (a), m is 1 or 2, and Z is a basic amino acid selected from: H, K, R, and a non-natural basic amino acid as defined herein, such as L-ornithine, preferably K or R, most preferably K.

Particularly preferred but non-limiting examples of such motifs are CC, KCC, KKCC (SEQ ID NO: 53), RCC, RRCC (SEQ ID NO: 54), RKCC (SEQ ID NO: 55), or KRCC (SEQ ID NO: 56).

(b) Zₘ-[CST]-Xₙ-C- or Zₘ-C-Xₙ-[CST]- as defined in aspect 2, wherein n is 1,

wherein X is any amino acid, preferably a basic amino acid selected from: H, K, R, and a non-natural basic amino acid such as L-ornithine, preferably K or R, most preferably K,
wherein m is an integer selected from 0 to 3,
wherein Z is any amino acid, preferably a basic amino acid selected from: H, K, R, and a non-natural basic amino acid as defined herein, such as L-ornithine, preferably K or R, most preferably K.

In preferred embodiments of motif (b), m is 1 or 2, and Z is a basic amino acid selected from: H, K, R, and a non-natural basic amino acid as defined herein, such as L-ornithine, preferably K or R, most preferably K.

Particularly preferred but non-limiting examples of such motifs are CRC, CKC, KCXC (SEQ ID NO: 57), KKCXC (SEQ ID NO: 58), RCXC (SEQ ID NO: 59), RRCXC (SEQ ID NO: 60), RKCXC (SEQ ID NO: 61), KRCXC (SEQ ID NO: 52), KCKC (SEQ ID NO: 63), KKCKC (SEQ ID NO: 64), KCRC (SEQ ID NO: 65), KKCRC (SEQ ID NO: 66), RCRC (SEQ ID NO: 67), RRCRC (SEQ ID NO: 68), RKCKC (SEQ ID NO: 69), or KRCKC (SEQ ID NO: 70).

(c) Zₘ-[CST]-Xₙ-C- or Zₘ-C-Xₙ-[CST]- as defined in aspect 2, wherein n is 2, thereby creating an internal X¹X² amino acid couple within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2.

In preferred embodiments, m is 1 and Z is a basic amino acid selected from: H, K, or R, or a non-natural basic amino acid as defined herein, such as L-ornithine, preferably K or R, most preferably K.

In preferred embodiments X¹ and X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a specific embodiment, at least one of X¹ or X² in said motif is a basic amino acid selected from: H, K, or R, or a non-natural basic amino acid as defined herein, such as L-ornithine. In another specific embodiment, at least one of X¹ or X² in said motif is P or Y. Specific examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH.

Particularly preferred motifs of this type are HCPYC, KCPYC, RCPYC, HCGHC, KCGHC, and RCGHC (corresponding to SEQ ID NO: 71 to 76).

(d) Zₘ-[CST]-Xₙ-C- or Zₘ-C-Xₙ-[CST]- as defined in aspect 2, wherein n is 3, thereby creating an internal X¹X²X³ amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2.

In some embodiments, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine.

Specific examples of the internal X¹X²X³ amino acid stretch within the oxidoreductase motif are: XPY, PXY, and PYX, wherein X can be any amino acid, preferably a basic amino acid such as K, R, or H, or a non-natural basic amino acid such as L-ornithine.

Non-limiting examples are:

KPY, RPY, HPY, GPY, APY, VPY, LPY, IPY, MPY, FPY, WPY, PPY, SPY, TPY, CPY, YPY, NPY, QPY, DPY, EPY, and KPY; or

PKY, PRY, PHY, PGY, PAY, PVY, PLY, PlY, PMY, PFY, PWY, PPY, PSY, PTY, PCY, PYY, PNY, PQY, PDY, PEY, and PLY; or

PYK, PYR, PYH, PYG, PYA, PYV, PYL, PYI, PYM, PYF, PYW, PYP, PYS, PYT, PYC, PYY, PYN, PYQ, PYD, PYE, and PYL;

XHG, HXG, and HGX, wherein X can be any amino acid, such as in:

KHG, RHG, HHG, GHG, AHG, VHG, LHG, IHG, MHG, FHG, WHG, PHG, SHG, THG, CHG, YHG, NHG, QHG, DHG, EHG, and KHG; or

HKG, HRG, HHG, HGG, HAG, HVG, HLG, HIG, HMG, HFG, HWG, HPG, HSG, HTG, HCG, HYG, HNG, HQG, HDG, HEG, and HLG; or

HGK, HGR, HGH, HGG, HGA, HGV, HGL, HGI, HGM, HGF, HGW, HGP, HGS, HGT, HGC, HGY, HGN, HGQ, HGD, HGE, and HGL;

XGP, GXP, and GPX, wherein X can be any amino acid, such as in:

KGP, RGP, HGP, GGP, AGP, VGP, LGP, IGP, MGP, FGP, WGP, PGP, SGP, TGP, CGP, YGP, NGP, QGP, DGP, EGP, and KGP; or

GKP, GRP, GHP, GGP, GAP, GVP, GLP, GIP, GMP, GFP, GWP, GPP, GSP, GTP, GCP, GYP, GNP, GQP, GDP, GEP, and GLP; or

GPK, GPR, GPH, GPG, GPA, GPV, GPL, GPI, GPM, GPF, GPW, GPP, GPS, GPT, GPC, GPY, GPN, GPQ, GPD, GPE, and GPL;

XGH, GXH, and GHX, wherein X can be any amino acid, such as in:

KGH, RGH, HGH, GGH, AGH, VGH, LGH, IGH, MGH, FGH, WGH, PGH, SGH, TGH, CGH, YGH, NGH, QGH, DGH, EGH, and KGH; or

GKH, GRH, GHH, GGH, GAH, GVH, GLH, GIH, GMH, GFH, GWH, GPH, GSH, GTH, GCH, GYH, GNH, GQH, GDH, GEH, and GLH; or

GHK, GHR, GHH, GHG, GHA, GHV, GHL, GHI, GHM, GHF, GHW, GHP, GHS, GHT, GHC, GHY, GHN, GHQ, GHD, GHE, and GHL;

XGF, GXF, and GFX, wherein X can be any amino acid, such as in:

KGF, RGF, HGF, GGF, AGF, VGF, LGF, IGF, MGF, FGF, WGF, PGF, SGF, TGF, CGF, YGF, NGF, QGF, DGF, EGF, and KGF; or

GKF, GRF, GHF, GGF, GAF, GVF, GLF, GIF, GMF, GFF, GWF, GPF, GSF, GTF, GCF, GYF, GNF, GQF, GDF, GEF, and GLF; or

GFK, GFR, GFH, GFG, GFA, GFV, GFL, GFI, GFM, GFF, GFW, GFP, GFS, GFT, GFC, GFY, GFN, GFQ, GFD, GFE, and GFL;

XRL, RXL, and RLX, wherein X can be any amino acid, such as in:

KRL, RRL, HRL, GRL, ARL, VRL, LRL, IRL, MRL, FRL, WRL, PRL, SRL, TRL, CRL, YRL, NRL, QRLRL, DRL, ERL, and KRL; or

GKF, GRF, GHF, GGF, GAF, GVF, GLF, GIF, GMF, GFF, GWF, GPF, GSF, GTF, GCF, GYF, GNF, GQF, GDF, GEF, and GLF; or

RLK, RLR, RLH, RLG, RLA, RLV, RLL, RLI, RLM, RLF, RLW, RLP, RLS, RLT, RLC, RLY, RLN, RLQ, RLD, RLE, and RLL;

XHP, HXP, and HPX, wherein X can be any amino acid, such as in:

KHP, RHP, HHP, GHP, AHP, VHP, LHP, IHP, MHP, FHP, WHP, PHP, SHP, THP, CHP, YHP, NHP, QHP, DHP, EHP, and KHP; or

HKP, HRP, HHP, HGP, HAF, HVF, HLF, HIF, HMF, HFF, HWF, HPF, HSF, HTF, HCF, HYP, HNF, HQF, HDF, HEF, and HLP; or

HPK, HPR, HPH, HPG, HPA, HPV, HPL, HPI, HPM, HPF, HPW, HPP, HPS, HPT, HPC, HPY, HPN, HPQ, HPD, HPE, and HPL;

Particularly preferred examples are: CRPYC, KCRPYC, KHCRPYC, RCRPYC, HCRPYC, CPRYC, KCPRYC, RCPRYC, HCPRYC, CPYRC, KCPYRC, RCPYRC, HCPYRC, CKPYC, KCKPYC, RCKPYC, HCKPYC, CPKYC, KCPKYC, RCPKYC, HCPKYC, CPYKC, KCPYKC, RCPYKC, and HCPYKC (corresponding to SEQ ID NO: 83 to 107).

(e) Zₘ-[CST]-Xₙ-C- or Zₘ-C-Xₙ-[CST]- as defined in aspect 2, wherein n is 4, thereby creating an internal X¹X²X³X⁴ amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In a specific embodiment, at least one of X¹, X², X³ or X⁴ in said motif is a basic amino acid selected from: H, K, or R, or a non-natural basic amino acid as defined herein.

Specific examples are: LAVL (SEQ ID NO: 108), TVQA (SEQ ID NO: 109) or GAVH (SEQ ID NO: 110) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 112 to 122); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

(f) Zₘ-[CST]-Xₙ-C- or Zₘ-C-Xₙ-[CST]- as defined in aspect 2, wherein n is 5, thereby creating an internal X¹X²X³X⁴X⁵ (SEQ ID NO: 125) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid selected from: H, K, or R, or a non-natural basic amino acid as defined herein.

Specific examples are: PAFPL (SEQ ID NO: 123) or DQGGE (SEQ ID NO: 124) and their variants such as: X¹AFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 138 to 143), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein.

(g) Zₘ-[CST]-Xₙ-C- or Zₘ-C-Xₙ-[CST]- as defined in aspect 2, wherein n is 6, thereby creating an internal X¹X²X³X⁴X⁵X⁶ (SEQ ID NO: 137) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or R, most preferably K. Preferred are motifs wherein m is 1 or 2. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T.

In a specific embodiment, at least one of X¹, X², X³ X⁴, X⁵ or X⁶ in said motif is a basic amino acid selected from: H, K, or R, or a non-natural basic amino acid as defined herein.

Specific examples are: DIADKY (SEQ ID NO: 136) or variants thereof such as: X¹IADKY, DX²ADKY, DIX³DKY, DIAX⁴KY, DIADX⁵Y, or DIADKX⁶ (corresponding to SEQ ID NO: 138 to 143), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein.

(h) Zₘ-[CST]-Xₙ-C- or Zₘ-C-Xₙ-[CST]-, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group (SEQ ID NO: 144 to 163).

In preferred embodiments of such a motif, n is 2, and m is 0, wherein the internal X¹X², each individually, can be any amino acid selected from the group consisting of: G, A, V, L, I, M, F, W, P, S, T, C, Y, N, Q, D, E, K, R, and H, or non-natural amino acids.

Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).

The oxidoreductase motif is placed either immediately adjacent to the epitope sequence within the peptide of the invention, or is separated from the T or NKT cell epitope by a linker. More particularly, the linker comprises an amino acid sequence of between 0 and 7 amino acids, that is 0, 1, 2, 3, 4, 5, 6, 7, 8, 9 or 10 amino acids. Most particularly, the linker comprises an amino acid sequence of between 0 and 4 amino acids, that is 0, 1, 2, 3, or 4 amino acids. Alternatively, a linker may comprise 5, 6, 7, 8, 9 or 10 amino acids.

Apart from a peptide linker other organic compounds can be used as linker to link the parts of the peptide to each other (e.g. the oxidoreductase motif to the T or NKT cell epitope sequence).

The peptides of the present invention can further comprise additional short amino acid sequences N or C-terminally of the (artificial) sequence comprising the T or NKT cell epitope and the oxidoreductase motif. Such an amino acid sequence is generally referred to herein as a ‘flanking sequence’. A flanking sequence can be positioned between the epitope and an endosomal targeting sequence and/or between the oxidoreductase motif and an endosomal targeting sequence. In further embodiments, not comprising an endosomal targeting sequence, a short amino acid sequence may be present N and/or C terminally of the oxidoreductase motif and/or epitope sequence in the peptide. More particularly a flanking sequence is a sequence of between 1 and 7 amino acids, most particularly a sequence of 2 amino acids.

The sequence comprising the T cell epitope and the reducing compound within the peptide can be further linked to an amino acid sequence (or another organic compound) that facilitates uptake of the peptide into late endosomes for processing and presentation within MHC class II or CD1d determinants. The late endosome targeting is mediated by signals present in the cytoplasmic tail of proteins and correspond to well-identified peptide motifs. The late endosome targeting is mediated by signals present in the cytoplasmic tail of proteins and correspond to well-identified peptide motifs such as the dileucine-based [DE]XXXL[LI] (SEQ ID NO: 192) or DXXLL motif (SEQ ID NO: 193) (e.g. DXXXLL, SEQ ID NO 194)), the tyrosine-based YXXO motif or the so-called acidic cluster motif (SEQ ID NO: 195). The symbol 0 represents amino acid residues with a bulky hydrophobic side chains such as Phe, Tyr and Trp. The late endosome targeting sequences allow for processing and efficient presentation of the antigen-derived T cell epitope by MHC class II or CD1d molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

The term “fumarate-related disease” encompasses all disorders or diseases that benefit from the treatment with fumarate. Preferred examples of such diseases or disorders are auto-immune disorders, demyelinating diseases, transplant rejection and cancer. Preferred examples of such diseases and disorders are: Multiple Sclerosis (MS), psoriasis, Neuromyelitis optica (NMO), Rheumatoid Arthritis (RA), polyarthritis, asthma, atopic dermatitis, scleroderma, ulcerative colitis, juveline diabetes, thyreoiditis, Grave's disease, Systemic Lupus Erythromatosis (SLE), Sjögren syndrome, anemia perniciosa, chronic active hepatitis, transplant rejection and cancer. Preferred examples are MOG autoantigen-related diseases and disorders such as MS and NMO.

The term “demyelination” as used within the framework of demyelinating diseases or disorders herein refers to damaging and/or degradation of myelin sheaths that surround axons of neurons which has as a consequence the formation of lesions or plaques. Due to demyelination, the signal conduction along the affected nerves is impaired, and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paresthesias, ocular paralysis, impaired muscle coordination, muscle weakness, loss of sensation, impaired vision, neurological symptoms, unsteady way of walking (gait), spastic paraparesis, incontinence, hearing problems, speech problems, and others. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). MS is considered in the art a demyelinating disorder of the central nervous system (Lubetzki and Stankoff. (2014). Handb Clin Neurol. 122, 89-99). Other specific but non-limiting examples of such demyelinating diseases and disorders include: neuromyelitis optica (NMO), acute inflammatory demyelinating polyneuropathy (AIDP), Chronic inflammatory demyelinating polyneuropathy (CIDP), acute transverse myelitis, progressive multifocal leucoencephalopathy (PML), acute disseminated encephalomyelitis (ADEM) or other hereditary demyelinating disorders.

The term “Multiple Sclerosis”, abbreviated herein and in the art as “MS”, indicates an autoimmune disorder affecting the central nervous system. MS is considered the most common non-traumatic disabling disease in young adults (Dobson and Giovannoni, (2019) Eur. J. Neurol. 26(1), 27-40), and the most common autoimmune disorder affecting the central nervous system (Berer and Krishnamoorthy (2014) FEBS Lett. 588(22), 4207-4213). MS may manifest itself in a subject by a large number of different symptoms ranging from physical over mental to psychiatric problems. Typical symptoms include blurred or double vision, muscle weakness, blindness in one eye, and difficulties in coordination and sensation. In most cases, MS may be viewed as a two-stage disease, with early inflammation responsible for relapsing-remitting disease and delayed neurodegeneration causing non-relapsing progression, i.e. secondary and primary progressive MS. Although progress is being made in the field, a conclusive underlying cause of the disease remains hitherto elusive and over 150 single nucleotide polymorphisms have been associated with MS susceptibility (International Multiple Sclerosis Genetics Consortium Nat Genet. (2013). 45(11):1353-60). Vitamin D deficiency, smoking, ultraviolet B (UVB) exposure, childhood obesity and infection by Epstein-Barr virus have been reported to contribute to disease development (Ascherio (2013) Expert Rev Neurother. 13(12 Suppl), 3-9).

Hence, MS can be regarded as a single disease existing within a spectrum extending from relapsing (wherein inflammation is the dominant feature) to progressive (neurodegeneration dominant). Therefore it is evident that the term Multiple sclerosis as used herein encompasses any type of Multiple Sclerosis belonging to any kind of disease course classification. In particular the invention is envisaged to be a potent treatment strategy patient diagnosed with, or suspected of having clinically Isolated Syndrome (CIS), relapse-remitting MS (RRMS), secondary progressive MS (SPMS), primary progressive MS (PPMS), and even MS-suspected radiology isolated syndrome (RIS). While strictly not considered a disease course of MS, RIS is used to classify subjects showing abnormalities on the Magnetic Resonance Imaging (MRI) of brain and/or spinal cord that correspond to MS lesions and cannot be prima facie explained by other diagnoses. CIS is a first episode (by definition lasting for over 24 hours) of neurologic symptoms caused by inflammation and demyelination in the central nervous system. In accordance with RIS, CIS classified subjects may or may not continue to develop MS, with subjects showing MS-like lesions on a brain MRI more likely to develop MS. RRMS is the most common disease course of MS with 85% of subjects having MS being diagnosed with RRMS. RRMS diagnosed patients are a preferred group of patients in view of the current invention. RRMS is characterized by attacks of new or increasing neurologic symptoms, alternatively worded relapses or exacerbations. In RRMS, said relapses are followed by periods or partial or complete remission of the symptoms, and no disease progression is experienced and/or observed in these periods of remission. RRMS may be further classified as active RRMS (relapses and/or evidence of new MRI activity), non-active RRMS, worsening RRMS (increasing disability over a specified period of time after a relapse, or not worsening RRMS. A portion of RRMS diagnosed subject will progress to the SPMS disease course, which is characterized by a progressive worsening of neurologic function, i.e. an accumulation of disability, overtime. SPMS subclassifications can be made such as active (relapses and/or new MRI activity), not active, progressive (disease worsening over time), or non-progressive SPMS. Finally, PPMS is an MS disease course characterized by worsening of neurologic function and hence an accumulation of disability from the onset of symptoms, without early relapse or remission. Further PPMS subgroups can be formed such as active PPMS (occasional relapse and/or new MRI activity), non-active PPMS, progressive PPMS (evidence of disease worsening over time, regardless of new MRI activity) and non-progressive PPMS. In general, MS disease courses are characterized by substantial intersubject variability in terms of relapse and remission periods, both in severity (in case of relapse) and duration.

Several disease modifying therapies are available for MS, and therefore the current invention may be used as alternative treatment strategy, or in combination with these existing therapies. Non-limiting examples of active pharmaceutical ingredients include interferon beta-1a, interferon beta-1b, glatiramer acetate, glatiramer acetate, peginterferon beta-1a, teriflunomide, fingolimod, cladribine, siponimod, dimethyl fumarate, diroximel fumarate, ozanimod, alemtuzumab, mitoxantrone, ocrelizumab, and natalizumab. Alternatively, the invention may be used in combination with a treatment or medication aiming to relapse management, such as but not limited to methylprednisolone, prednisone, and adrenocorticotropic hormone(s) (ACTH). Further, the invention may be used in combination with a therapy aiming to alleviate specific symptoms. Non-limiting examples include medications aiming to improve or avoid symptoms selected from the group consisting of: bladder problems, bowel dysfunction, depression, dizziness, vertigo, emotional changes, fatigue, itching, pain, sexual problems, spasticity, tremors, and walking difficulties.

MS is characterized by three intertwined hallmark characteristics: 1) lesion formation in the central nervous system, 2) inflammation, and 3) degradation of myelin sheaths of neurons. Despite traditionally being considered a demyelinating disease of the central nervous system and white matter, more recently reports have surfaced that demyelination of the cortical and deep gray matter may exceed white matter demyelination (Kutzelnigg et al. (2005). Brain. 128(11), 2705-2712). Two main hypotheses have been postulated as to how MS is caused at the molecular level. The commonly accepted “outside-in hypothesis” is based on the activation of peripheral autoreactive effector CD4+ T cells which migrate to the central nervous system and initiate the disease process. Once in the central nervous system, said T cells are locally reactivated by APCs and recruit additional T cells and macrophages to establish inflammatory lesions. Noteworthy, MS lesions have been shown to contain CD8+ T cells predominantly found at the lesion edges, and CD4+ T cells found more central in the lesions. These cells are thought to cause demyelination, oligodendrocyte destruction, and axonal damage, leading to neurologic dysfunction. Additionally, immune-modulatory networks are triggered to limit inflammation and to initiate repair, which results in at least partial remyelination reflected by clinical remission. Nonetheless, without adequate treatment, further attacks often lead to progression of the disease.

MS onset is believed to originate well before the first clinical symptoms are detected, as evidenced by the typical occurrence of apparent older and inactive lesions on the MRI of patients. Due to advances in the development of diagnostic methods, MS can now be detected even before a clinical manifestation of the disease (i.e. pre-symptomatic MS). In the context of the invention, “treatment of MS” and similar expressions envisage treatment of, and treatment strategies for, both symptomatic and pre-symptomatic MS. In particular, when the immunogenic peptides and/or resulting cytolytic CD4+ T cells are used for treating a pre-symptomatic MS patient, the disease is halted at such an early stage that clinical manifestations may be partially, or even completely avoided. MS wherein the subject is not fully responsive to a treatment of interferon beta is also encompassed within the term “MS”.

The term “Neuromyelitis Optica” or “NMO” and “NMO Spectrum Disorder (NMOSD)”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May; 13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-MOG antibodies, it is considered that anti-MOG antibodies may trigger an attack on the myelin sheath resulting in demyelination. The cause of NMO in the majority of cases is due to a specific attack on auto-antigens. Up to a third of subjects may be positive for auto-antibodies directed against a component of myelin called myelin oligodendrocyte glycoprotein (MOG). People with anti-MOG related NMO similarly have episodes of transverse myelitis and optic neuritis. Particularly envisaged within the framework of this application is NMO induced by MOG autoantigens and/or caused by anti-MOG antibodies.

The term “Rheumatoid Arthritis” or “RA” is an autoimmune, inflammatory disease that causes pain, swelling, stiffness, and loss of function in various joints (most commonly in the hands, wrists, and knees). The respective joint's lining becomes inflamed, leading to tissue damage, as well as chronic pain, unsteadiness, and deformity. There is generally a bilateral/symmetrical pattern of disease progression (e.g., both hands or both knees are affected). RA can also affect extra-articular sites, including the eyes, mouth, lungs, and heart. Patients can experience an acute worsening of their symptoms (called a flare) but with early intervention and appropriate treatment, symptoms can be ameliorated for a certain duration (reviewed by Sana Iqbal et al., 2019, US Pharm. 2019; 44(1)(Specialty&Oncology suppl):8-11). The antigens attacked by the immune system and responsible for the disease are diverse but some examples are: GRP78, HSP60, 60 kDa chaperonin 2, Gelsolin, Chitinase-3-like protein 1, Cathepsin S, Serum albumin, and Cathepsin D.

The term “Psoriasis” refers to a chronic inflammatory skin disease with a strong genetic predisposition and autoimmune pathogenic traits. The worldwide prevalence is about 2%, but varies according to regions. It shows a lower prevalence in Asian and some African populations, and up to 11% in Caucasian and Scandinavian populations. The dermatologic manifestations of psoriasis are varied; psoriasis vulgaris is also called plaque-type psoriasis, and is the most prevalent type. The terms psoriasis and psoriasis vulgaris are used interchangeably in the scientific literature; nonetheless, there are important distinctions among the different clinical subtypes. Psoriasis Vulgaris (about 90% of psoriasis cases) is a chronic plaque-type psoriasis. The classical clinical manifestations are sharply demarcated, erythematous, pruritic plaques covered in silvery scales. The plaques can coalesce and cover large areas of skin. Common locations include the trunk, the extensor surfaces of the limbs, and the scalp. Other types are: Inverse Psoriasis, also called flexural psoriasis, affects intertriginous locations, and is characterized clinically by slightly erosive erythematous plaques and patches; Guttate Psoriasis, which is a variant with an acute onset of small erythematous plaques. It usually affects children or adolescents, and is often triggered by group-A streptococcal infections of tonsils. About one-third of patients with guttate psoriasis will develop plaque psoriasis throughout their adult life; Pustular psoriasis characterized by multiple, coalescing sterile pustules. Pustular psoriasis can be localized or generalized. Two distinct localized phenotypes have been described: psoriasis pustulosa palmoplantaris (PPP) and acrodermatitis continua of Hallopeau. Both of them affect the hands and feet; PPP is restricted to the palms and soles, and ACS is more distally located at the tips of fingers and toes, and affects the nail apparatus. Generalized pustular psoriasis presents with an acute and rapidly progressive course characterized by diffuse redness and subcorneal pustules, and is often accompanied by systemic symptoms. The hallmark of psoriasis is sustained inflammation that leads to uncontrolled keratinocyte proliferation and dysfunctional differentiation. The histology of the psoriatic plaque shows acanthosis (epidermal hyperplasia), which overlies inflammatory infiltrates composed of dermal dendritic cells, macrophages, T cells, and neutrophils. Neovascularization is also a prominent feature. The inflammatory pathways active in plaque psoriasis and the rest of the clinical variants overlap, but also display discrete differences that account for the different phenotype and treatment outcomes. (reviewed by Rendon and Schskel, Int J Mol Sci. 2019 March; 20(6): 1475).

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence. The selection of the antigen whereon the epitope of the immunogenic or tolerogenic peptide as described herein is designed will depend on the fumarate-related disease.

Exemplary antigens can be:
myelin antigens, neuronal antigens, and astrocyte-derived antigens, for example: Myelin Oligodendrocyte Glycoprotein (MOG), Myelin basic protein (MBP), Proteolipid protein (PLP), Oligodendrocyte-specific protein (OSP), myelin-associated antigen (MAG), myelin-associated oligodendrocyte basic protein (MOBP), and 2′,3′-cyclic-nucleotide 3′-phosphodiesterase (CNPase), S100P protein or transaldolase H autoantigens in case of MS (Riedhammer and Weissert, 2015; Front Immunol. 2015; 6: 322), preferably MOG, MBP, PLP and MOBP.
ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37, preferably LL37 (Jason E. Hawkes et al., 2017: Current Dermatology Reports volume 6, pages 104-112).
N-acetylglucosamine-6-sulfatase (GNS), filamin A (FLNA), vinculin, GRP78, HSP60, 60 kDa chaperonin 2, Gelsolin, Chitinase-3-like protein 1, Cathepsin S, Serum albumin, and Cathepsin D in Rheumatoid Arthritis.
allergens such as those derived from pollen, spores, dust mites, and pet dander in case of asthma.
tumour or cancer associated antigens such as oncogenes, proto-oncogenes, viral proteins, surviving factors or clonotypic or idiotypic determinants in case of cancer.

Specific examples are: MAGE (melanoma-associated gene) products were shown to be spontaneously expressed by tumour cells in the context of MHC class I determinants, and as such, recognised by CD8+ cytolytic T cells. However, MAGE-derived antigens, such as MAGE-3, are also expressed in MHC class II determinants and CD4+ specific T cells have been cloned from melanoma patients (Schutz et al. (2000) Cancer Research 60: 6272-6275; Schuler-Thurner et al. (2002) J. Exp. Med. 195: 1279-1288). Peptides presented by MHC class II determinants are known in the art. Other examples include the gp100 antigen expressed by the P815 mastocytoma and by melanoma cells (Lapointe (2001; J. Immunol. 167: 4758-4764; Cochlovius et al. (1999) Int. J. Cancer, 83: 547-554). Proto-oncogenes include a number of polypeptides and proteins which are preferentially expressed in tumours cells, and only minimally in healthy tissues. Cyclin D1 is cell cycle regulator which is involved in the G1 to S transition. High expression of cyclin D1 has been demonstrated in renal cell carcinoma, parathyroid carcinomas and multiple myeloma. A peptide encompassing residues 198 to 212 has been shown to carry a T cell epitope recognised in the context of MHC class II determinants (Dengiel et al. (2004) Eur. J. of Immunol. 34: 3644-3651). Survivin is one example of a factor inhibiting apoptosis, thereby conferring an expansion advantage to survivin-expressing cells. Survivin is aberrantly expressed in human cancers of epithelial and hematopoietic origins and not expressed in healthy adult tissues except the thymus, testis and placenta, and in growth-hormone stimulated hematopoietic progenitors and endothelial cells. Interestingly, survivin-specific CD8+ T cells are detectable in blood of melanoma patients. Survivin is expressed by a broad variety of malignant cell lines, including renal carcinoma, breast cancer, and multiple myeloma, but also in acute myeloid leukemia, and in acute and chronic lymphoid leukemia (Schmidt (2003) Blood 102: 571-576). Other examples on inhibitors of apoptosis are Bcl2 and spi6. Idiotypic determinants are presented by B cells in follicular lymphomas, multiple myeloma and some forms of leukemia, and by T cell lymphomas and some T cell leukemias. Idiotypic determinants are part of the antigen-specific receptor of either the B cell receptor (BCR) or the T cell receptor (TCR). Such determinants are essentially encoded by hypervariable regions of the receptor, corresponding to complementarity-determining regions (CDR) of either the VH or VL regions in B cells, or the CDR3 of the beta chain in T cells. As receptors are created by the random rearrangement of genes, they are unique to each individual. Peptides derived from idiotypic determinants are presented in MHC class II determinants (Baskar et al. (2004) J. Clin. Invest. 113: 1498-1510). Some tumours are associated with expression of virus-derived antigens. Thus, some forms of Hodgkin disease express antigens from the Epstein-Barr virus (EBV). Such antigens are expressed in both class I and class II determinants. CD8+ cytolytic T cells specific for EBV antigens can eliminate Hodgkin lymphoma cells (Bollard et al. (2004) J. Exp. Med. 200: 1623-1633). Antigenic determinants such as LMP-1 and LMP-2 are presented by MHC class II determinants.
transplant-specific antigens in case of transplant rejection, which will obviously be dependent on the type of tissue or organ being transplanted. Examples can be tissues such as cornea, skin, bones (bone chips), vessels or fascia; organs such as kidney, heart, liver, lungs, pancreas or intestine; or even individual cells such as pancreatic islet cells, alpha cells, beta cells, muscle cells, cartilage cells, heart cells, brain cells, blood cells, bone marrow cells, kidney cells and liver cells. Specific exemplary antigens involved in transplantation rejection are minor histocompatibility antigens, major histocompatibility antigens or tissue-specific antigens. Where the alloantigenic protein is a major histocompatibility antigen, this is either an MHC class I-antigen or an MHC class II-antigen. An important point to keep in mind is the variability of the mechanisms by which alloantigen-specific T cells recognize cognate peptides at the surface of APC. Alloreactive T cells can recognize either alloantigen-determinants of the MHC molecule itself, an alloantigen peptide bound to a MHC molecule of either autogenic or allogeneic source, or a combination of residues located within the alloantigen-derived peptide and the MHC molecule, the latter being of autogenic or allogeneic origin. Examples of minor histocompatibility antigens are those derived from proteins encoded by the HY chromosome (H-Y antigens), such as Dby. Other examples can be found in, for instance, Goulmy E, Current Opinion in Immunology, vol 8, 75-81, 1996 (see Table 3 therein in particular). It has to be noted that many minor histocompatibility antigens in man have been detected via their presentation into MHC class I determinants by use of cytolytic CD8+ T cells. However, such peptides are derived by the processing of proteins that also contain MHC class II restricted T cell epitopes, thereby providing the possibility of designing peptides of the present invention. Tissue-specific alloantigens can be identified using the same procedure. One example of this is the MHC class I restricted epitope derived from a protein expressed in kidneys but not in spleen and capable of eliciting CD8+ T cells with cytotoxic activity on kidney cells (Poindexter et al, Journal of Immunology, 154: 3880-3887, 1995).

The term Myelin Oligodendrocyte Glycoprotein refers to the human protein encoded by the MOG gene. The terms MOG (protein) or Myelin Oligodendrocyte Glycoprotein as used herein are defined by the amino acid sequence corresponding to NCBI Gene 4340, and UniProtKB identifier Q16653 (MOG_HUMAN) (SEQ ID NO: 184):

MASLSRPSLPSCLCSFLLLLLLQVSSSYAGQFRVIGPRHPIRALVGDEV
ELPCRISPGKNATGMEVGWYRPPFSRVVHLYRNGKDQDGDQAPEYRGRT
ELLKDAIGEGKVTLRIRNVRFSDEGGFTCEFRDHSYQEEAAMELKVEDP
FYWVSPGVLVLLAVLPVLLLQITVGLIFLCLQYRLRGKLRAEIENLHRT
FDPHFLRVPCWKITLFVIVPVLGPLVALIICYNWLHRRLAGQFLEELRN
PF

Myelin Oligodendrocyte Glycoprotein is a membrane protein expressed on the oligodendrocyte cell surface and the outermost surface of myelin sheaths and is a primary target antigen involved in immune-mediated demyelination. The protein may be involved in completion and maintenance of the myelin sheath and in cell-cell communication. Alternatively spliced transcript variants encoding different isoforms have been identified. The MOG epitopes envisaged for incorporation in the immunogenic or tolerogenic peptides of the invention may thus be epitopes that are present in the canonical MOG amino acid sequence (SEQ ID NO: 184), and/or one or more MOG protein isoforms. A suitable MOG epitope in the context of the invention is a MOG epitope comprising, or consisting of, FLRVPCWKI (SEQ ID NO: 164). The SEQ ID NO: 164 portion of the human and mouse MOG protein is characterized by 100% sequence identity. Alternatively worded, SEQ ID NO: 164 can be retrieved in both the human and mouse MOG protein. Alternatively a point mutation may be introduced in the MOG epitope SEQ ID NO: 164 to form the amino acid sequence FLRVPSWKI (SEQ ID NO: 165), which is a preferred MOG epitope in the context of the invention. Another suitable MOG epitope in the context of the invention is a MOG epitope comprising, or consisting of, VVHLYRNGK (SEQ ID NO: 170). The SEQ ID NO: 164 portion of the human and mouse MOG protein is characterized by 100% sequence identity. The terms “treatment” or “treating” encompasses the therapeutic treatment of an already developed fumarate-related disease or disorder. The term “prevention” refers to prophylactic or preventative measures, wherein the aim is to prevent or lessen the chances of incidence of the fumarate-related disease or disorder. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. The terms “treatment” or “treating” can also mean prolonging survival as compared to expected survival if not receiving treatment.

The immunogenic peptide as defined herein may be adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including, but not limited to, oral, intranasal or intramuscular. Also, the number of injections and the amount injected can vary depending on the severity of the condition to be treated, and other parameters, such as the age, body weight, general health, sex and diet of the patient. Further, other adjuvants than alum can be used, provided they facilitate peptide presentation in MHC-class II or CD1d and T or NKT cell activation. Thus, while it is possible for the immunogenic peptides to be administered without any adjuvant, they typically are presented as pharmaceutical formulations. The formulations, both for veterinary and for human use, comprise at least one immunogenic peptide, as above described, together with one or more pharmaceutically acceptable carriers.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment. Such polynucleotides or nucleic acids molecules may be readily prepared using an automated synthesisers and the well-known codon-amino acid relationship of the genetic code. Such polynucleotides or nucleic acids may be incorporated into expression vectors, including plasmids, which are adapted for the expression of the polynucleotide or nucleic acid and production of the polypeptide in a suitable host such as bacterium, e.g. Escherichia coli, yeast cell, human cell, animal cell or plant cell.

For therapeutic means, polynucleotides encoding the immunogenic or tolerogenic peptides disclosed herein can be part of an expression system, cassette, plasmid or vector system such as viral and non-viral expression systems. Viral vectors known for therapeutic purposes are adenoviruses, adeno-associated viruses (AAVs), lentiviruses, and retroviruses. Non-viral vectors can be used as well and non-limiting examples include: transposon-based vector systems such as those derived from Sleeping Beauty (SB) or PiggyBac (PB). Nucleic acids can also be delivered through other carriers such as but not limited to nanoparticles, cationic lipids, liposomes etc.

The term “pharmaceutically acceptable carrier” as used herein with respect to the dosage forms comprising the tolerogenic peptide or immunogenic peptide as defined herein means any material or substance with which the immunogenic or tolerogenic peptide is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic or tolerogenic peptide in the pharmaceutical formulation. The pharmaceutically acceptable carrier may be a solid or a liquid or a gas which has been compressed to form a liquid, i.e. the formulations can suitably be used as concentrates, emulsions, solutions, granulates, dusts, sprays, aerosols, suspensions, ointments, creams, tablets, pellets or powders. Suitable pharmaceutical carriers for use in the pharmaceutical formulations of the peptide are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical formulations of the immunogenic or tolerogenic peptide may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the immunogenic or tolerogenic peptide.

The pharmaceutical composition may comprise a therapeutically or prophylactically effective amount of the or each immunogenic or tolerogenic peptide and optionally a pharmaceutically acceptable carrier, diluent or excipient. Also, in the pharmaceutical compositions of the present invention, the or each immunogenic or tolerogenic peptide may be admixed with any suitable binder(s), lubricant(s), suspending agent(s), coating agent(s), or solubilising agent(s).

Suitable surface-active agents for use in the pharmaceutical formulations of the immunogenic or tolerogenic peptide, also known as emulgent or emulsifier, non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardio-lipin, dioctanylphosphatidylcholine, dipalmitoylphoshatidylcholine and their mixtures. Suitable non-ionic surfactants include polyethoxylated and poly-propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol—polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C₈C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

The pharmaceutical dosage forms or pharmaceutical formulations of the immunogenic or tolerogenic peptide suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the immunogenic or tolerogenic peptide in the required amount in the appropriate solvent with various of the other ingredients enumerated above, as required, followed by filtered sterilization. Generally, dispersions are prepared by incorporating the sterilized immunogenic or tolerogenic peptide into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the immunogenic or tolerogenic peptide plus any additional desired ingredient from a previously sterile-filtered solution thereof.

Upon formulation, pharmaceutical preparations as defined herein or the peptides as defined herein or the fumarate compound as defined herein can be administered in a manner compatible with the dosage formulation and in such amount as is therapeutically effective.

Administration of the tolerogenic peptide should preferably be done in soluble form in the absence of adjuvant. The tolerogenic peptides of the invention or the pharmaceutical composition comprising such as defined herein is preferably administered through mucosal delivery such as through nasal, oral, buccal, pulmonary, ocular, vaginal, or rectal delivery; or through, intradermal administration, transdermal administration or subcutaneously injection. Studies have shown that tolerogenic peptides, when given in soluble form intraperitoneally (i.p.), intravenously (i.v.) or intranasally (i.n.) or orally can induce T cell tolerance (Anderton and Wraith (1998) as above; Liu and Wraith (1995) as above; Metzler and Wraith (1999) Immunology 97: 257-263). A dose escalation protocol may be followed, where a plurality of doses of the tolerogenic peptide is given to the patient in increasing concentrations as has been successfully tested in case of bee venom allergy (Muller et al (1998) J. Allergy Clin Immunol. 101: 747-754 and Akdis et al (1998) J. Clin. Invest. 102: 98-106) and as disclosed in WO2018127828. In one embodiment, said tolerogenic peptide can be formulated according to techniques known in the art and exemplified for example in patent application WO2013160865A1.

The immunogenic peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The immunogenic peptide according to the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single dose or in 2, 3, 4, 5, or more doses, either simultaneously or consecutively.

In certain embodiments, the treatment can be repeated several times throughout the disease of the subject. Such consecutive treatments can be done daily, or with an intermission of 1 to 10 days, such as for example every 5 to 9 days such as about every 7 days.

Alternatively, said treatment can be repeated weekly, biweekly, monthly, bimonthly, or every three to four months.

Exemplary non-limiting administration schemes are the following:
A low dose scheme comprising the SC administration of 50 μg of peptide in two separate injections of 25 μg each (100 μL each) followed by three consecutive injections of 25 μg of immunogenic peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of immunogenic peptide as two separate injections of 37.5 μg each (150 μL each).
A high dose scheme comprising the SC administration of 450 μg of peptide in two separate injections of 225 μg each (900 μL each) followed by three consecutive administrations of 225 μg of immunogenic peptide as two separate injections of 112.5 μg each (450 μL each).

Other exemplary non-limiting administration schemes are the following:
A dose scheme comprising 6 SC administration 2 weeks apart of 450 μg of immunogenic peptide in two separate injections of 225 μg each.
A dose scheme comprising 6 SC administration 2 weeks apart SC of 1350 μg of immunogenic peptide in two separate injections of 675 μg each.

The immunogenic or tolerogenic peptide formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

Administration of the immunogenic peptide should preferably be done in soluble form in the presence of adjuvant.

Immunogenic or tolerogenic peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, microspheres, microemulsions, nanoparticles, nanocapsules etc. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention is further illustrated by the following examples, which do not limit the scope of the invention in any way.

### EXAMPLES

Example 1: Effect of the prophylactic administration of an immunogenic peptide comprising a MOG₃₅₋₅₅ MHCII T cell epitope and a HCPYC oxidoreductase motif in combination with dimethyl fumarate (BG-12, TECFIDERA™) on experimental auto-immune encephalomyelitis (EAE) development in mice.

Groups of Mice and Dosing

The study used a total of 64 female C57BL/6 mice (Taconic Biosciences, 10 weeks old on Day 0). Mice were acclimated for 8 days prior to the first injection. Mice were assigned to groups in a balanced manner to achieve similar average weight across the groups at the start of the study. Table 1 below shows the treatment administered to each group.

TABLE 1
Treatment regimen

| | # | Treatment 1 | Treatment 1 | Treatment 2 | Treatment 2 | |
|---|---|---|---|---|---|---|
| Group | animals | (s.c.) | dosing days | (p.o.) | dosing days | Purpose |
| 1 | 16 | Saline | −21, −14, −7 | Vehicle | −28 to 27 | Negative |
| | | | | | | control |
| 2 | 16 | IMCY- | −21, −14, −7 | Vehicle | −28 to 27 | Test |
| | | 0189/Alum | | | | |
| 3 | 16 | Saline | −21, −14, −7 | BG-12 | 0 to 27 | Positive |
| | | | | | | control |
| 4 | 16 | IMCY- | −21, −14, −7 | BG-12 | 0 to 27 | Test |
| | | 0189/Alum | | | | |

Treatment 1 was administered once on each of the days indicated in Table 1, s.c., at a volume of 0.05 mL/site, each mouse receiving injection at two sites, for a total of 0.1 mL/mouse/dosing day, corresponding to 100 μg of peptide.

Treatment 2 was administered p.o., BID, at a volume of 10 mL/kg, over the days indicated in Table 1. BG-12 was dosed at 100 mg/kg. All dosing was done at the same time (+/−1 hour) each dosing day. For the BID groups there were no less than 10 and no more than 14 hours between doses.

Compound Preparation

For Treatment 1, 0.9% NaCl solution was prepared at each dosing day. Lyophilized immunogenic peptide IMCY-0189 with the sequence HCPYCGWYRSPFSRVVHLYR (SEQ ID NO: 185), comprising an oxidoreductase motif HCPYC (SEQ ID NO: 71), a linker GW, a murine Myelin Oligodendrocyte Glycoprotein (MOG₃₅₋₅₅) MHCII T cell epitope YRSPFSRVV (SEQ ID NO: 169) and a flanking sequence HLYR (SEQ ID NO: 186) (Smart Bioscience) was solubilized immediately before use. Lyophilized IMCY-0189 was thawed at room temperature for 10 minutes, resuspended in Na Acetate buffer 50 mM pH 5.4 and incubated at room temperature for 5 minutes. Reconstituted peptide was then mixed with Imject™ Alum Adjuvant before injection.

For Treatment 2, vehicle was 0.5% HPMC, 0.2% Tween 20, and 50 mM citrate buffer at pH 4. BG-12 (or TECFIDERA™, from Santa Cruz Biotechnology, catalog number sc-239774) was prepared once a week. At each preparation the required amount of BG-12 was weighed out into a mortar and triturated with a pestle. Vehicle was then added in small increments and mixed until the final volume was reached. The material was then vortexed and sonicated in a water bath until a homogeneous suspension was obtained. Formulated BG-12 was stored at 4° C., stirring continuously.

EAE Induction

EAE was induced in all mice as follows:
Day 0, Hour 0—Immunization with a peptide corresponding to the amino acids 35-55 of MOG (MOG₃₅₋₅₅)/CFA
Day 0, Hour 2—Injection of pertussis toxin
Day 1, Hour 0-2^{nd} injection of pertussis toxin (24 hours after initial immunization).

Mice were injected subcutaneously at two sites in the back with the emulsion component (containing MOG₃₅₋₅₅) of Hooke Kit™ MOG₃₅₋₅₅/CFA Emulsion PTX, catalog number EK-2110 (lot #127, Hooke Laboratories, Lawrence Mass.). One site of injection was in the area of upper back, approximately 1 cm caudal of the neck line. The second site was in the area of lower back, approximately 2 cm cranial of the base of the tail. The injection volume was 0.1 mL at each site. Each mouse received 200 μg of MOG₃₅₋₅₅.

Within 2 hours of the injection of emulsion, and then again 24 hours after the injection of emulsion, the pertussis toxin component of the kit was administered intraperitoneally. The pertussis toxin (lot #1008, Hooke Laboratories) was administered at 100 ng/dose for both injections and the volume of each injection was 0.1 mL.

EAE Scoring

Animals were scored daily starting from Day 7 to the end of the study. Scoring was performed blind, by a person unaware of both treatment and of previous scores for each mouse. EAE was scored on the scale 0 to 5 as shown in Table 2 below. In-between scores were assigned when the clinical signs fell between two above defined scores.

TABLE 2
EAE scoring criteria

| Score | Clinical observations |
|---|---|
| 0 | No obvious changes in motor functions |
| 1 | Limp tail |
| 2 | Limp tail and weakness of hind legs |
| 3 | Limp tail and complete paralysis of hind legs |
| 4 | Limp tail, complete hind leg and partial front leg paralysis |
| 5 | Complete hind and complete front leg paralysis, or death due |
| | to paralysis |

Plasma Neurofilaments Levels Determination

On Day 28, blood was collected from all mice into tubes containing K2EDTA and mixed gently. Blood was then centrifuged at ˜10000 g for 5 minutes. Plasma was transferred into Eppendorf tubes and stored at −80° C. until shipment to Quanterix™. Plasma Neurofilament light (NF-L) protein levels were quantified using Simoa® NF-light Advantage kit, a digital immunoassay for the quantitative determination of NF-L in serum, plasma and CSF. The used antibodies (Uman Diagnostics, Umea Sweden) also cross react with murine, bovine and macaque NF-L epitopes and as such, this assay can be used for research with these species. All samples were tested in duplicate at a dilution factor of 40×.

Terminal Collection

At the end of the study, all mice were euthanized, and spines were collected and placed in 10% buffered formalin for histological analysis.

Histology

For each spine, one H&E stained slide and one anti-MBP stained slide were prepared and analyzed. Each slide contained a section with samples from lumbar, thoracic and cervical of spinal cord (3 samples). All analysis was performed by a pathologist blinded to the experimental groups and all clinical readouts.

Inflammatory foci of approximately 20 cells were counted in each H&E stained section. When inflammatory infiltrates consisted of more than 20 cells, an estimate was made of how many foci of 20 cells were present.

Demyelination was scored in each anti-MBP (using immunohistochemistry) stained section. In anti-MBP sections, demyelination is observed as conspicuous unstained areas in white matter tracts and is associated with presence of large vacuoles. The demyelination score represents an estimate of demyelinated area for each section as follows:

0—no demyelination (less than 5% demyelinated area)
1—5 to 20% demyelinated area
2—20 to 40% demyelinated area
3—40 to 60% demyelinated area
4—60 to 80% demyelinated area
5—80 to 100% demyelinated area

Statistical Analysis

AUC, MMS, inflammation and demyelination, and NF-L levels quantification data were analyzed by performing Ordinary one-way ANOVA. Adjustment for multiplicity was performed using Holm-Sidak's method. Significant differences are referred as follows: *p<0.05, **p<0.01, ***p<0.001, ****p<0.0001.

Results and Interpretation of Data

EAE Scoring

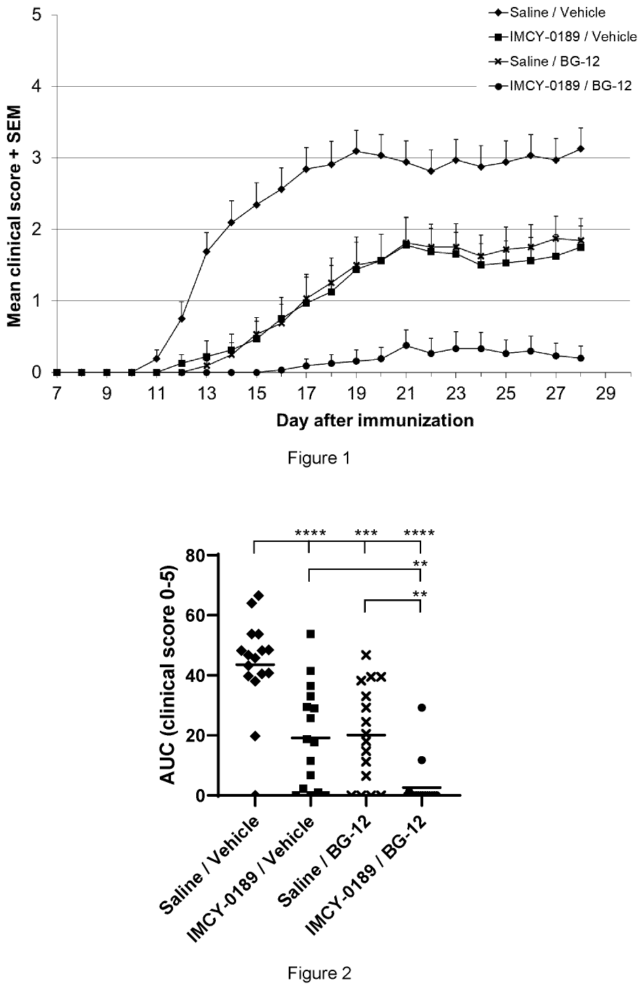

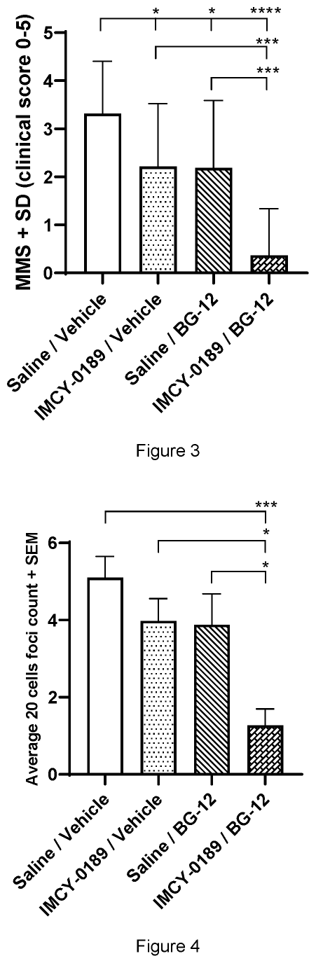

EAE development was evaluated by comparing clinical EAE readouts for all groups to the negative control (Saline/Vehicle) group. EAE scoring, AUC (area under the curve) and MMS (mean maximal score) are presented in FIGS. 1, 2 and 3.

Mice of the Saline/Vehicle group (negative control) developed typical EAE for this model. Two (2) mice in this group died due to severe EAE.

Mice treated with BG-12 (Saline/BG-12 group) showed postponed disease onset and reduced end score, and statistically significant reduced AUC and MMS compared to the negative control group. No mice died in this group.

Mice treated with the IMCY-0189 (IMCY-0189/Vehicle group) also showed postponed disease onset and reduced end score, and statistically significant reduced AUC and MMS compared to the negative control group. These clinical results appeared similar to those observed with BG-12 treatment. Two (2) mice died in this group. One (1) mouse died due to severe EAE. The death of the other mouse did not appear to be due to EAE, and therefore that mouse was excluded from analysis.

All clinical readouts (disease onset, end score, AUC and MMS) of mice treated with both IMCY-0189 and BG-12 were statistically significantly improved, compared to the negative control group, and also especially compared to IMCY-0189 only (IMCY-0189/Vehicle group) or BG-12 only (Saline/BG-12 group) treated mice. One (1) mouse died in this group, but the death of this mouse did not appear to be due to EAE, and therefore it was excluded from analysis. Interaction between both IMCY-0189 and BG-12 treatments was analyzed by performing a Two-way ANOVA. A tendency towards synergy was assessed by a p-value close to 0.2 (0.2373) on MMS data.

Histology

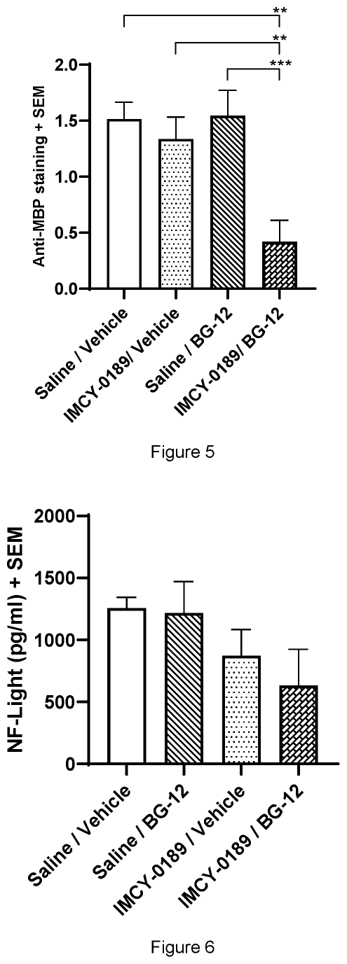

Histological readouts were evaluated by comparing inflammation and demyelination levels for all groups to the negative control (Saline/Vehicle) group. Inflammation and demyelination data are presented in FIGS. 4 and 5.

Histological results for the Saline/Vehicle group (negative control) were consistent with the clinical findings and as expected for this model.

Mice treated with BG-12 (Saline/BG-12 group, positive control) showed similar level of demyelination compared to the negative control group, and reduced level of inflammation, although not statistically significant.

Similarly, mice treated with the IMCY-0189 (IMCY-0189/Vehicle group) showed reduced level of both inflammation and demyelination, although not statistically significant.

Histological readouts of mice treated with IMCY-0189 and BG-12 were statistically significantly improved, compared to the negative control group, and also especially compared to IMCY-0189 only (IMCY-0189/Vehicle group) or BG-12 only (Saline/BG-12 group) treated mice. Interaction between both IMCY-0189 and BG-12 treatments was analyzed by performing a Two-way ANOVA. A synergistic effect between both treatments was assessed by an interaction p-value <0.05 on demyelination data.

Plasma Neurofilaments Levels

Neurofilament light (NF-L) is a 68 kDa cytoskeletal filament protein that is expressed in neurons, as one of the major components of the neuronal cytoskeleton that provide structural support for the axon. Neurofilaments can be released following axonal damage or neuronal degeneration. NF-L has been shown to associate with neurodegenerative diseases such as multiple sclerosis.

The experiment described in table 1 was repeated to evaluate axonal damage by comparing NF-L levels for all groups to the negative control (Saline/Vehicle) group. Data are presented in FIG. 6.

NF-L levels for the Saline/Vehicle group (negative control) were consistent with the clinical findings and as expected for this model.

Mice treated with BG-12 (Saline/BG-12 group, positive control) showed similar NF-L levels compared to the negative control group.

Mice treated with the IMCY-0189 (IMCY-0189/Vehicle group), as well as mice treated with both IMCY-0189 and BG-12 (IMCY-0189/BG-12 group) showed reduced NF-L levels.

Example 2: Effect of the therapeutic administration of an immunogenic peptide comprising a MOG₃₅₋₅₅ MHCII T cell epitope linked or not to an HCPYC oxidoreductase motif in combination with dimethyl fumarate (BG-12, TECFIDERA™) on experimental auto-immune encephalomyelitis (EAE) development in mice.

Groups of Mice and Dosing

The study used a total of 96 female C57BL/6 mice (Taconic Biosciences, 10 weeks old on Day 0). Mice were acclimated for 14 days prior to the first injection. Mice were assigned to groups in a balanced manner to achieve similar average weight across the groups at the start of the study. Table 3 below shows the treatment administered to each group.

TABLE 3
Treatment regimen

| | # | Treatment 1 | Treatment 1 | Treatment 2 | Treatment 2 | |
|---|---|---|---|---|---|---|
| Group | animals | (s.c.) | dosing days | (p.o.) | dosing days | Purpose |
| 1 | 16 | Saline | 4, 9, 14, 19 | Vehicle | 0 to 27 | Negative |
| | | | | | | control |
| 2 | 16 | Saline | 4, 9, 14, 19 | BG-12 | 0 to 27 | Positive |
| | | | | | | control |
| 3 | 16 | IMCY- | 4, 9, 14, 19 | Vehicle | 0 to 27 | Test |
| | | 0189/Alum | | | | |
| 4 | 16 | IMCY- | 4, 9, 14, 19 | BG-12 | 0 to 27 | Test |
| | | 0189/Alum | | | | |
| 5 | 16 | MOG₃₅₋₅₅/ | 4, 9, 14, 19 | Vehicle | 0 to 27 | Test |
| | | Alum | | | | |
| 6 | 16 | MOG₃₅₋₅₅/ | 4, 9, 14, 19 | BG-12 | 0 to 27 | Test |
| | | Alum | | | | |

Treatment 1 was administered once on each of the days indicated in Table 3, s.c., at a volume of 0.05 mL/site, each mouse receiving injection at two sites, for a total of 0.1 mL/mouse/dosing day. IMCY-0189 or MOG₃₅₋₅₅ peptide total dose was 30 μg per administration.

Treatment 2 was administered p.o., BID, at a volume of 10 mL/kg, over the days indicated in Table 3. BG-12 was dosed at 100 mg/kg.

All dosing were done at the same time (+/−1 hour) each dosing day. For the BID groups there were no less than 10 and no more than 14 hours between doses.

Compound Preparation

For Saline treatment, 0.9% NaCl solution was prepared at each dosing day.

Treatment 1—MOG₃₅₋₅₅ Peptide Preparation:

Lyophilized mouse MOG₃₅₋₅₅ peptide with the sequence MEVGWYRSPFSRVVHLYRNGK (SEQ ID NO:205), comprising the mouse Myelin Oligodendrocyte Glycoprotein (MOG₃₅₋₅₅) MHCII T cell epitope YRSPFSRVV (SEQ ID NO:169), was solubilized immediately before use. Lyophilized MOG₃₅₋₅₅ peptide was thawed at room temperature for 10 minutes, resuspended in Na Acetate buffer 50 mM NaCl 0.9% pH 5.4 and incubated at room temperature for 10 minutes. Reconstituted MOG₃₅₋₅₅ peptide was then mixed with Imject™ Alum Adjuvant before injection. Note that the positions 35 to 55 refer to the mature protein, i.e. after cleavage of the signal peptide (AA 1-29), defined by SEQ ID NO: 208. In the full-length amino acid sequence of MOG (SEQ ID NO: 184), said peptide would be at positions 64-84.

Treatment 1—IMCY-0189 Peptide Preparation:

IMCY-0189 has the sequence described in example 1. Lyophilized IMCY-0189 was thawed at room temperature for 10 minutes, resuspended in Na Acetate buffer 50 mM NaCl 0.9% pH 5.4 and incubated at room temperature for 10 minutes. Reconstituted peptide was then mixed with Imject™ Alum Adjuvant before injection.

Treatment 2

For Treatment 2, vehicle was 0.5% HPMC, 0.2% Tween 20, and 50 mM citrate buffer at pH 4. BG-12 (Santa Cruz Biotechnology, sc-239774) and was prepared at least every 2 weeks. At each preparation the required amount of BG-12 was weighed out into a mortar and triturated with a pestle. Vehicle was then added in small increments and mixed until the final volume was reached. The material was then vortexed and sonicated in a water bath until a homogeneous suspension was obtained. Formulated BG-12 was stored at 4° C., stirring continuously.

EAE induction, scoring and statistical analysis were performed as described in example 1.

Results and Interpretation of Data

EAE Scoring

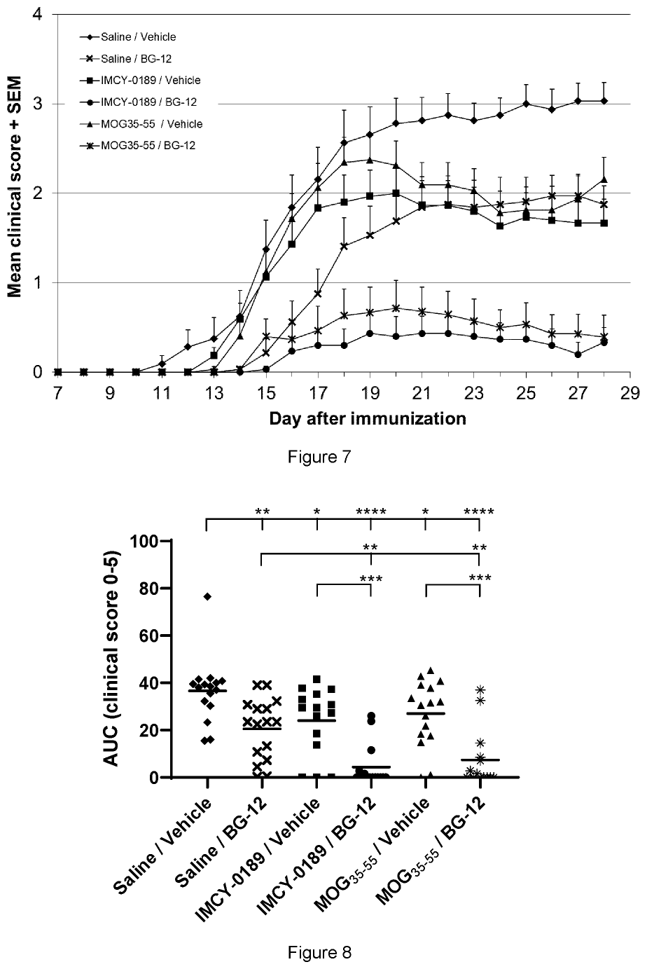

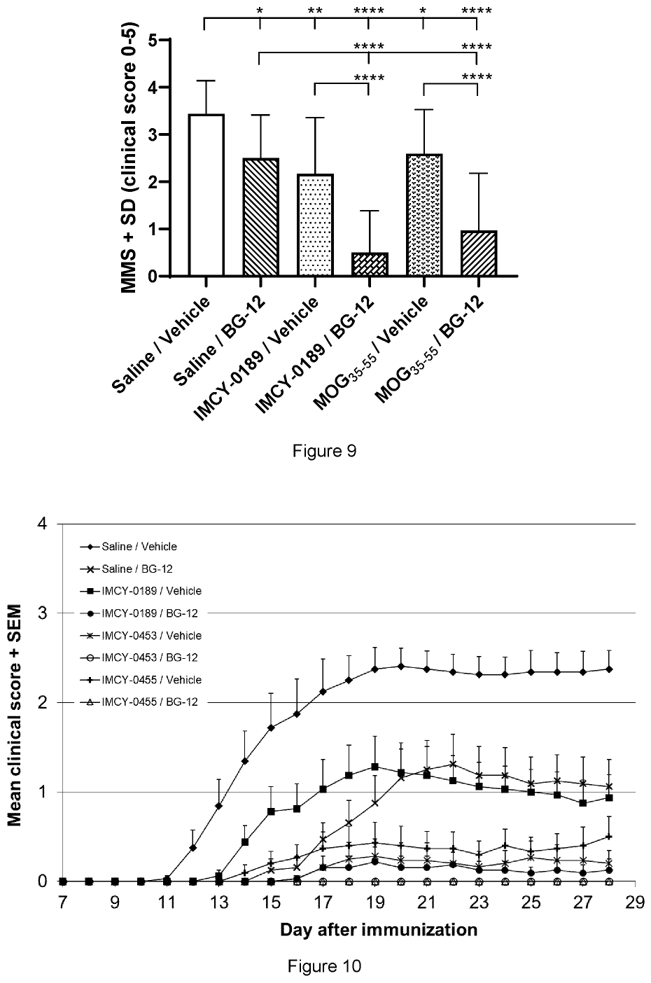

EAE development was evaluated by comparing clinical EAE readouts for all groups to the negative control (Saline/Vehicle) group. EAE scoring, AUC (area under the curve) and MMS (mean maximal score) are presented in FIGS. 7, 8 and 9.

Mice of the Saline/Vehicle group (negative control) developed typical EAE for this model. Two (2) mice in this group died due to severe EAE.

Mice treated with BG-12 (Saline/BG-12 group) showed postponed disease onset and reduced end score, and statistically significant reduced AUC and MMS compared to the negative control group. No mice died in this group.

Mice treated with the MOG₃₅₋₅₅ peptide (MOG₃₅₋₅₅/Vehicle group) or IMCY-0189 (IMCY-0189/Vehicle group) also showed postponed disease onset and reduced end score, and statistically significant reduced AUC and MMS compared to the negative control group. No mice died in the MOG₃₅₋₅₅/Vehicle group, while one (1) mouse died in the IMCY-0189/Vehicle group, but the death of that mouse did not appear to be due to EAE, and therefore it was excluded from analysis.

All clinical readouts (disease onset, end score, AUC and MMS) of mice treated with both the MOG₃₅₋₅₅ peptide and BG-12 or with both IMCY-0189 and BG-12 were statistically significantly improved compared to mice treated with peptides only or BG-12 only. Two (2) mice and one (1) mouse died in the MOG₃₅₋₅₅/Vehicle group and the IMCY-0189/Vehicle group respectively, but the death of these mice did not appear to be due to EAE, and therefore they were excluded from analysis. Interactions between both IMCY-0189 and BG-12 treatments or both MOG₃₅₋₅₅ and BG-12 treatments were analyzed by performing Two-way ANOVA. A tendency towards synergy was assessed by p-values close to 0.1 (0.1306 and 0.1574 respectively) on MMS data.

Example 3: Effect of the therapeutic administration of an immunogenic peptide comprising a MOG₃₅₋₅₅ MHCII T cell epitope linked to a KCRC (SEQ ID NO: 65) or KCRPYC (SEQ ID NO: 84) oxidoreductase motif in combination with dimethyl fumarate (BG-12, TECFIDERA™) on experimental auto-immune encephalomyelitis (EAE) development in mice.

Groups of Mice and Dosing

The study used a total of 128 female C57BL/6 mice (Taconic Biosciences, 9 weeks old on Day 0). Mice were acclimated for 7 days prior to the first injection. Mice were assigned to groups in a balanced manner to achieve similar average weight across the groups at the start of the study. Table 4 below shows the treatment administered to each group.

TABLE 4
Treatment regimen

| | # | Treatment 1 | Treatment 1 | Treatment 2 | Treatment 2 | |
|---|---|---|---|---|---|---|
| Group | animals | (s.c.) | dosing days | (p.o.) | dosing days | Purpose |
| 1 | 16 | Saline | 4, 9, 14, 19 | Vehicle | 0 to 27 | Negative |
| | | | | | | control |
| 2 | 16 | Saline | 4, 9, 14, 19 | BG-12 | 0 to 27 | Positive |
| | | | | | | control |
| 3 | 16 | IMCY- | 4, 9, 14, 19 | Vehicle | 0 to 27 | Test |
| | | 0189/Alum | | | | |
| 4 | 16 | IMCY- | 4, 9, 14, 19 | BG-12 | 0 to 27 | Test |
| | | 0189/Alum | | | | |
| 5 | 16 | IMCY- | 4, 9, 14, 19 | Vehicle | 0 to 27 | Test |
| | | 0453/Alum | | | | |
| 6 | 16 | IMCY- | 4, 9, 14, 19 | BG-12 | 0 to 27 | Test |
| | | 0453/Alum | | | | |
| 7 | 16 | IMCY- | 4, 9, 14, 19 | Vehicle | 0 to 27 | Test |
| | | 0455/Alum | | | | |
| 8 | 16 | IMCY- | 4, 9, 14, 19 | BG-12 | 0 to 27 | Test |
| | | 0455/Alum | | | | |

Treatment 1 was administered once on each of the days indicated in Table 4, s.c., at a volume of 0.05 mL/site, each mouse receiving injection at two sites, for a total of 0.1 mL/mouse/dosing day. IMCY-0189 (CXXC oxidoreductase motif, IMCY-0453 (CXC oxidoreductase motif) or IMCY-0455 (CXXXC oxidoreductase motif) peptide total dose was 30 μg per administration.

Treatment 2 was administered p.o., BID, at a volume of 10 mL/kg, over the days indicated in Table 4. BG-12 was dosed at 100 mg/kg.

All dosing were done at the same time (+/−1 hour) each dosing day. For the BID groups there were no less than 10 and no more than 14 hours between doses.

Compound Preparation

For Saline treatment, 0.9% NaCl solution was prepared at each dosing day.

Treatment 1—IMCY-0189 Peptide Preparation:

IMCY-0189 has the sequence described in example 1 and was prepared as described in example 2.

Treatment 1—IMCY-0453 and IMCY-0455 Peptides Preparation:

Lyophilized immunogenic peptide IMCY-0453 with the sequence KCRCGWYRSPFSRVVHLYR (SEQ ID NO: 266), comprising an oxidoreductase motif KCRC (SEQ ID NO: 65), a linker GW, a murine Myelin Oligodendrocyte Glycoprotein (MOG₃₅₋₅₅) MHCII T cell epitope YRSPFSRVV (SEQ ID NO: 169) and a flanking sequence HLYR (SEQ ID NO: 186) (Smart Bioscience) was solubilized immediately before use. Lyophilized IMCY-0453 was thawed at room temperature for 10 minutes, resuspended in Na Acetate buffer 50 mM NaCl 0.9% pH 5.4 and incubated at room temperature for 10 minutes. Reconstituted peptide was then mixed with Imject™ Alum Adjuvant before injection.

Lyophilized immunogenic peptide IMCY-0455 with the sequence KCRPYCGWYRSPFSRVVHLYR (SEQ ID NO: 268), comprising an oxidoreductase motif KCRPYC (SEQ ID NO: 84), a linker GW, a murine Myelin Oligodendrocyte Glycoprotein (MOG₃₅₋₅₅) MHCII T cell epitope YRSPFSRVV (SEQ ID NO: 169) and a flanking sequence HLYR (SEQ ID NO: 186) (Smart Bioscience) was solubilized immediately before use. Lyophilized IMCY-0455 was thawed at room temperature for 10 minutes, resuspended in Na Acetate buffer 50 mM NaCl 0.9% pH 5.4 and incubated at room temperature for 10 minutes. Reconstituted peptide was then mixed with Imject™ Alum Adjuvant before injection.

Treatment 2 was prepared as described in example 2.

Serum Neurofilaments Levels Determination

On Day 28, blood was collected from all mice into gel clot activator tubes and allowed to clot at room temperature for ˜30 minutes. Blood is then centrifuged at ˜10000 g for 5 minutes. Serum was transferred into Eppendorf tubes and stored at −80° C. until shipment to Quanterix™. Serum Neurofilament light (NF-L) protein levels were quantified using Simoa® NF-light Advantage kit as described in example 1.

EAE induction, scoring and statistical analysis were performed as described in example

2. Results and Interpretation of Data

EAE Scoring

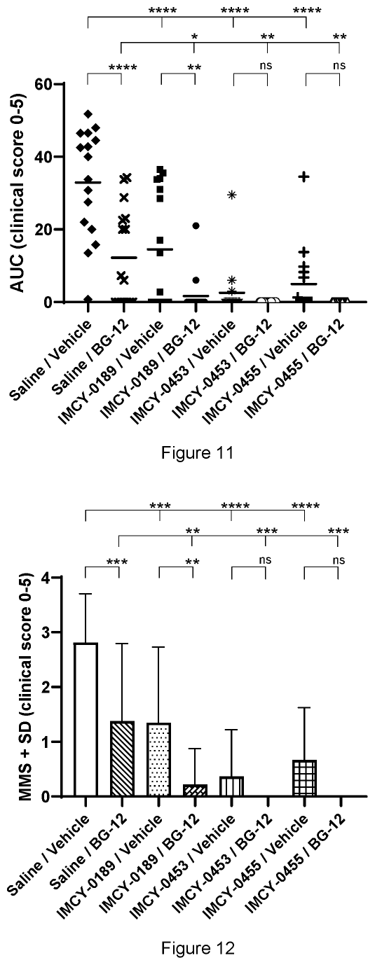

EAE development was evaluated by comparing clinical EAE readouts for all groups to the negative control (Saline/Vehicle) group. EAE scoring, AUC (area under the curve) and MMS (mean maximal score) are presented in FIGS. 10, 11 and 12.

Mice of the Saline/Vehicle group (negative control) developed somewhat milder EAE but still within the expected range for this model. No mice died in this group.

Mice treated with BG-12 (Saline/BG-12 group) showed postponed disease onset and reduced end score, and statistically significant reduced AUC and MMS compared to the negative control group. Mice treated with IMCY-0189 (IMCY-0189/Vehicle group) showed similar profile. No mice died in these two groups.

All clinical readouts (disease onset, end score, AUC and MMS) of mice treated with IMCY-0453 or IMCY-0455 were statistically significantly improved as compared to the negative control group. Co-administration of BG-12 together with IMCY-0453 or IMCY-0455 appeared the most efficacious, with no mice in these groups developing EAE. One (1) mouse of the IMCY-453/Vehicle group, one (1) mouse of the IMCY-455/Vehicle group, and one (1) of the IMCY-453/BG-12 group died, but the death of these mice did not appear to be due to EAE, and therefore they were excluded from analysis.

Serum Neurofilaments Levels

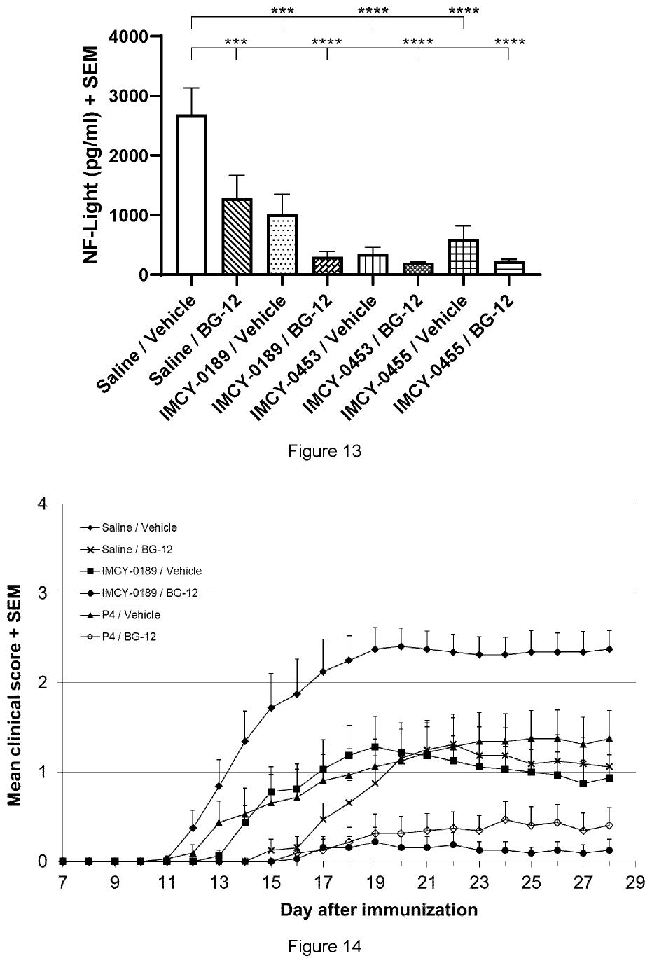

Axonal damage was also evaluated by comparing NF-L levels for all groups to the negative control (Saline/Vehicle) group. Data are presented in FIG. 13.

NF-L levels for the Saline/Vehicle group (negative control) were consistent with the clinical findings and as expected for this model.

Mice treated with the IMCY-0189, IMCY-0453 and IMCY-0455 orwith BG-12 (Saline/BG-12 group, positive control) showed statistically significant reduced NF-L levels compared to the negative control group.

NF-L levels of mice treated with both IMCY-0189, IMCY-0453, IMCY-0455 and BG-12 are nearly abolished as compared to the negative control group.

Example 4: Effect of the therapeutic administration of an immunogenic peptide comprising a human MOG₂₀₁₋₂₁₂ MHCII T cell epitope linked to a KHCPYC oxidoreductase motif in combination with dimethyl fumarate (BG-12, TECFIDERA™) on experimental auto-immune encephalomyelitis (EAE) development in mice.

Groups of Mice and Dosing

The study used a total of 96 female C57BL/6 mice (Taconic Biosciences, 9 weeks old on Day 0). Mice were acclimated for 7 days prior to the first injection. Mice were assigned to groups in a balanced manner to achieve similar average weight across the groups at the start of the study. Table 5 below shows the treatment administered to each group.

TABLE 5
Treatment regimen

| | # | Treatment 1 | Treatment 1 | Treatment 2 | Treatment 2 | |
|---|---|---|---|---|---|---|
| Group | animals | (s.c.) | dosing days | (p.o.) | dosing days | Purpose |
| 1 | 16 | Saline | 4, 9, 14, 19 | Vehicle | 0 to 27 | Negative |
| | | | | | | control |
| 2 | 16 | Saline | 4, 9, 14, 19 | BG-12 | 0 to 27 | Positive |
| | | | | | | control |
| 3 | 16 | IMCY- | 4, 9, 14, 19 | Vehicle | 0 to 27 | Test |
| | | 0189/Alum | | | | |
| 4 | 16 | IMCY- | 4, 9, 14, 19 | BG-12 | 0 to 27 | Test |
| | | 0189/Alum | | | | |
| 5 | 16 | P4/Alum | 4, 9, 14, 19 | Vehicle | 0 to 27 | Test |
| 6 | 16 | P4/Alum | 4, 9, 14, 19 | BG-12 | 0 to 27 | Test |

Treatment 1 was administered once on each of the days indicated in Table 5, s.c., at a volume of 0.05 mL/site, each mouse receiving injection at two sites, for a total of 0.1 mL/mouse/dosing day. IMCY-0189 or P4 peptide total dose was 30 μg per administration.

Treatment 2 was administered p.o., BID, at a volume of 10 mL/kg, over the days indicated in Table 5. BG-12 was dosed at 100 mg/kg.

All dosing were done at the same time (+/−1 hour) each dosing day. For the BID groups there were no less than 10 and no more than 14 hours between doses.

Compound Preparation

For Saline treatment, 0.9% NaCl solution was prepared at each dosing day.

Treatment 1—IMCY-0189 Peptide Preparation:

IMCY-0189 has the sequence described in example 1 and was prepared as described in example 2.

Treatment 1—P4 Peptide Preparation:

Lyophilized immunogenic peptide P4 with the sequence KHCPYCVRYFLRVPSWKITLFKK (SEQ ID NO: 176), comprising an oxidoreductase motif KHCPYC (SEQ ID NO: 77), a linker VRY, a human Myelin Oligodendrocyte Glycoprotein (MOG₂₀₁₋₂₁₂) MHCII T cell epitope FLRVPSWKI (SEQ ID NO: 165) and a flanking sequence TLFKK (SEQ ID NO: 267) (Smart Bioscience) was solubilized immediately before use. Lyophilized P4 was thawed at room temperature for 10 minutes, resuspended in Na Acetate buffer 50 mM NaCl 0.9% pH 5.4 and incubated at room temperature for 10 minutes. Reconstituted peptide was then mixed with Imject™ Alum Adjuvant before injection.

Treatment 2 was prepared as described in example 2.

Serum neurofilaments levels were quantified as described in example 3.

EAE induction, scoring and statistical analysis were performed as described in example 2.

Results and Interpretation of Data

EAE Scoring

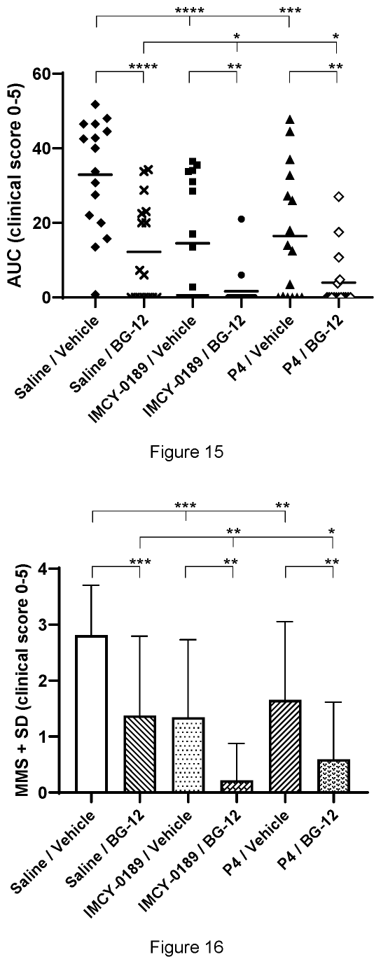

EAE development was evaluated by comparing clinical EAE readouts for all groups to the negative control (Saline/Vehicle) group. EAE scoring, AUC (area under the curve) and MMS (mean maximal score) are presented in FIGS. 14, 15 and 16.

Mice of the Saline/Vehicle group (negative control) developed somewhat milder EAE but still within the expected range for this model. No mice died in this group.

Mice treated with BG-12 (Saline/BG-12 group), IMCY-0189 (IMCY-0189/Vehicle group) and P4 (P4/Vehicle group) showed postponed disease onset and reduced end score, and statistically significant reduced AUC and MMS compared to the negative control group. No mice died in these three groups.

All clinical readouts (disease onset, end score, AUC and MMS) of mice treated with both IMCY-0189 and BG-12 or with both P4 and BG-12 were statistically significantly improved compared to the negative control group, and also especially compared to peptides only or BG-12 only treated mice. No mice died in these two groups. Interactions between both IMCY-0189 and BG-12 treatments or both P4 and BG-12 treatments were analyzed by performing Two-way ANOVA. A tendency towards synergy was assessed by p-values close to 0.2 (0.2340 and 0.2392 respectively) on AUC data.

Serum Neurofilaments Levels

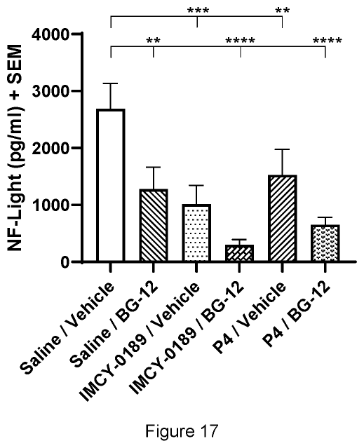

Axonal damage was also evaluated by comparing NF-L levels for all groups to the negative control (Saline/Vehicle) group. Data are presented in FIG. 17.

NF-L levels for the Saline/Vehicle group (negative control) were consistent with the clinical findings and as expected for this model.

Mice treated with the IMCY-0189, P4 or with BG-12 (Saline/BG-12 group, positive control) showed statistically significant reduced NF-L levels compared to the negative control group.

Mice treated with both IMCY-0189 and BG-12 or with both P4 and BG-12 showed highly statistically significant reduced NF-L levels compared to the negative control group.

### Standard Definitions and Abbreviations

AUC area under the curve
BG-12 dimethyl fumarate, DMF
BID twice daily
° C. degrees Celsius
CFA complete Freund's adjuvant
EAE experimental autoimmune encephalomyelitis
H&E hematoxylin and eosin
HPMC hydroxypropyl methylcellulose
MBP myelin basic protein
MMS mean maximum score
MOG myelin oligodendrocyte glycoprotein
Na sodium
NF-L Neurofilament light protein
p.o. oral
PTX pertussis toxin
s.c. subcutaneous
SD standard deviation
SEM standard error of the mean

1. A pharmaceutical kit comprising:
a) one or more dosage forms of a fumarate compound selected from the group consisting of: dimethyl fumarate (DMF), monomethyl fumarate (MMF), compounds that can be metabolized into MMF in vivo, and combinations thereof; and
b) one or more dosage forms of an immunogenic or tolerogenic peptide comprising a T cell epitope of an antigenic protein involved in a fumarate-related disease or disorder.

2. The pharmaceutical kit according to claim 1, wherein said peptide is an immunogenic peptide having an oxidoreductase motif linked to said T cell epitope, said oxidoreductase motif having a sequence of the general formula:
Zₘ-[CST]-Xₙ-C- (SEQ ID NO: 1 to 25) or Zₘ-C-Xₙ-[CST]- (SEQ ID NO: 26 to 50),
wherein n is an integer chosen from 2, 1, 3, or 0, wherein m is an integer selected from 0 to 3, wherein X is any amino acid, wherein Z is any amino acid, in which [CST] stands for any one of cysteine (C), serine (S), or threonine (T);
wherein said oxidoreductase motif and said T cell epitope are separated by a linker of between 0 and 7 amino acids,
wherein the hyphen (-) in said oxidoreductase motif indicates the point of attachment of the oxidoreductase motif to the N-terminal end of the linker or the epitope, or to the C-terminal end of the linker or the T cell epitope.

3. The pharmaceutical kit according to claim 1 or 2, wherein said fumarate compound is dimethyl fumarate, monomethyl fumarate, or a combination of dimethyl fumarate and monomethyl fumarate, or a prodrug, a deuterated form, a clathrate, a solvate, a tautomer, a stereoisomer, or a pharmaceutically acceptable salt thereof.

4. The pharmaceutical kit according to claim 1, 2 or 3, wherein said compound is selected from the group consisting of dimethyl fumarate (Formula (II) below), monomethyl fumarate (Formula (III) below), diroximel fumarate (Formula (IV) below), and tepilamide fumarate (Formula (V) below): 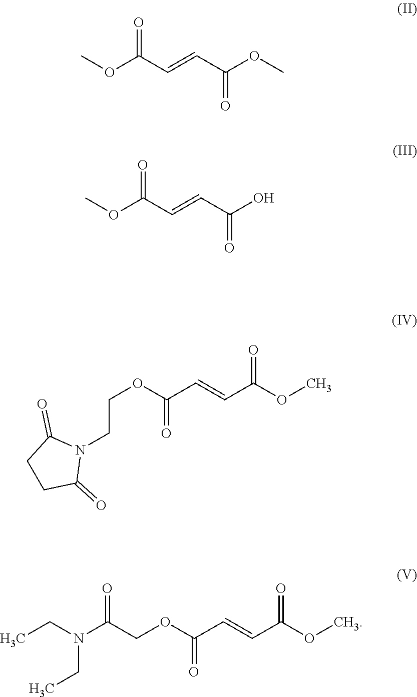
or a combination of any one or more thereof, or a deuterated form, a clathrate, a solvate, a tautomer, a stereoisomer, or a pharmaceutically acceptable salt of any one or more thereof, or a combination of any one of the foregoing.

5. The pharmaceutical kit according to any one of claims 1 to 4, wherein said fumarate-related disease or disorder is an auto-immune disorder, a demyelinating disorder, transplant rejection or cancer.

6. The pharmaceutical kit according to any one of claims 1 to 5, wherein said fumarate-related disease or disorder is MS and wherein said autoantigen is selected from the group consisting of: Myelin Oligodendrocyte Glycoprotein (MOG), Myelin basic protein (MBP), Proteolipid protein (PLP), myelin-associated antigen (MAG), Oligodendrocyte-specific protein (OSP), myelin-associated oligodendrocyte basic protein (MOBP), and 2′,3′-cyclic-nucleotide 3′-phosphodiesterase (CNPase), S1003 protein and transaldolase H; or
wherein said fumarate-related disease or disorder is NMO and wherein said autoantigen is MOG; or
wherein said fumarate-related disease or disorder is Psoriasis and wherein said autoantigen is selected from the group consisting of: ADAMTSL5, PLA2G4D, Keratin, such as Keratin 14 or Keratin 17, an antigen from Triticum aestivum, Pso p27, cathelicidin antimicrobial peptide, ceutrophil defensin 1 and LL37; or
wherein said fumarate-related disease or disorder is Rheumatoid Arthritis (RA) and wherein said antigenic protein is selected from the group comprising: GRP78, HSP60, 60 kDa chaperonin 2, Gelsolin, Chitinase-3-like protein 1, Cathepsin S, Serum albumin, vinculin, and Cathepsin D.

7. The pharmaceutical kit according to any one of claims 1 to 6, comprising an immunogenic peptide wherein the (auto)antigen involved in a fumarate-related disease or disorder does not naturally comprise a oxidoreductase motif within 11 amino acids N- or C-terminally adjacent to said epitope, and/or wherein said epitope does not naturally comprise an oxidoreductase motif in its sequence.

8. The pharmaceutical kit according to any one of claims 1 to 7, wherein the T-cell epitope is an MHC class II T-cell epitope or an NKT cell epitope.

9. The pharmaceutical kit according to any one of claims 1 to 8, wherein the T cell epitope is an NKT cell epitope has the amino acid motif [FWHY]-XX-[ILMV]-XX-[FWHY] (SEQ ID NO: 51), preferably the amino acid motif [FW]-XX-[ILMV]-XX-[FW] (SEQ ID NO: 52).

10. The pharmaceutical kit according to any one of claims 1 to 9, wherein the oxidoreductase motif in said immunogenic peptide is located N-terminally from the epitope.

11. The pharmaceutical kit according to any one of claims 1 to 10, wherein in said immunogenic peptide, the epitope has a length of between 7 and 30 amino acids, preferably between 7 and 25 amino acids, more preferably between 7 and 20 amino acids.

12. The pharmaceutical kit according to any one of claims 1 to 11, wherein said tolerogenic or immunogenic peptide has a length of between 9 and 50 amino acids, preferably between 9 and 40 amino acids, more preferably between 9 and 30 amino acids.

13. The pharmaceutical kit according to any one of claims 2 to 12, wherein in said immunogenic peptide, said oxidoreductase motif is selected from the following amino acid motifs:
Zₘ-[CST]-Xₙ-C- or Zₘ-C-Xₙ-[CST]-, (a)
wherein n is 0, and wherein m is 0, 1, or 2
wherein Z is a basic amino acid preferably selected from: H, K, R, and a non-natural basic amino acid as defined herein, such as L-ornithine, more preferably K or H;
Zₘ-[CST]-Xₙ-C- or Zₘ-C-Xₙ-[CST]-, (b)
wherein n is 1, wherein X is any amino acid, preferably a basic amino acid selected from: H, K, R, and a non-natural basic amino acid such as L-ornithine, preferably K or R,
wherein m is 0, 1, or 2,
wherein Z is a basic amino acid preferably selected from: H, K, R, and a non-natural basic amino acid as defined herein, such as L-ornithine, preferably K or H;
Zₘ-[CST]-Xₙ-C- or Zₘ-C-Xₙ-[CST]-, (c)
wherein n is 2, thereby creating an internal X¹X² amino acid couple within the oxidoreductase motif, wherein m is 0, 1, or 2, wherein Z is a basic amino acid preferably selected from: H, K, R, and a non-natural basic amino acid as defined herein, such as L-ornithine, preferably K or H; or
Zₘ-[CST]-Xₙ-C- or Zₘ-C-Xₙ-[CST]-, (d)
wherein n is 3, thereby creating an internal X¹X²X³ amino acid stretch within the oxidoreductase motif, wherein m is 0, 1, or 2, wherein Z is a basic amino acid preferably selected from: H, K, R, and a non-natural basic amino acid as defined herein, such as L-ornithine, preferably K or H.

14. The pharmaceutical kit according to any one of claims 1 to 13, wherein said immunogenic peptide has an oxidoreductase motif which comprises the sequence CC, KCC, RCC, CRC, CKC, KCRC (SEQ ID NO: 65), KCKC (SEQ ID NO: 63), RCKC (SEQ ID NO: 171), RCRC (SEQ ID NO: 67), CPYC (SEQ ID NO: 172), HCPYC (SEQ ID NO: 71), KCPYC (SEQ ID NO: 72), RCPYC (SEQ ID NO: 73), CRPYC (SEQ ID NO: 83), CPRYC (SEQ ID NO: 88), CPYRC (SEQ ID NO: 92), CKPYC (SEQ ID NO: 96), CPKYC (SEQ ID NO: 100), CPYKC (SEQ ID NO: 104), RCRPYC (SEQ ID NO: 86), RCPRYC (SEQ ID NO: 90), RCPYRC (SEQ ID NO: 94), RCKPYC (SEQ ID NO: 98), RCPKYC (SEQ ID NO: 102), RCPYKC (SEQ ID NO: 106), KCRPYC (SEQ ID NO: 84), KCPRYC (SEQ ID NO: 89), KCPYRC (SEQ ID NO: 93), KCKPYC (SEQ ID NO: 97), KCPKYC (SEQ ID NO: 101), or KCPYKC (SEQ ID NO: 105).

15. The pharmaceutical kit according to any one of claims 1 to 14, wherein said tolerogenic or immunogenic peptide comprises a T-cell epitope derived from the Myelin-oligodendrocyte glycoprotein (MOG) antigen amino acid sequence, preferably selected from the group comprising:
(SEQ ID NO: 200)
YRPPFSRVVHLYRNGKDQDGD,
(SEQ ID NO: 201)
RPPFSRVVHLYRNGK,
(SEQ ID NO: 202)
PFSRVVHLYRNGKDQ,
(SEQ ID NO: 203)
FSRVVHLYRNGKDQD,
(SEQ ID NO: 204)
SRVVHLYRNGKDQDG,
(SEQ ID NO: 164)
FLRVPCWKI,
(SEQ ID NO: 165)
FLRVPSWKI,
(SEQ ID NO: 170)
VVHLYRNGK,
(mouse SEQ ID NO: 205)
MEVGWYRSPFSRVVHLYRNGK,
(human SEQ ID NO: 206)
MEVGWYRPPFSRVVHLYRNGK,
(mouse SEQ ID NO: 169)
YRSPFSRVV,
and
(human SEQ ID NO: 168)
YRPPFSRVV,
or combinations thereof;
wherein said immunogenic peptide comprises a T-cell epitope derived from the myelin proteolipid protein (PLP) antigen amino acid sequence, preferably selected from the group comprising:
(SEQ ID NO: 209)
HEALTGTEKLIETYFSKNYQDYEYLI,
(SEQ ID NO: 210)
TWTTCQSIAFPSKTSASIGSLCADARMY,
(SEQ ID NO: 211)
GVLPWNAFPGKVCGSNLLSICKTAEFQM,
(SEQ ID NO: 212)
LTGTEKLIETYFSKNYQDY,
(SEQ ID NO: 213)
WTTCQSIAFPSKTSASIGS,
(SEQ ID NO: 214)
VLPWNAFPGKVCGSN,
(SEQ ID NO: 215)
LTGTEKLIETYFSKN,
(SEQ ID NO: 216)
TEKLIETYFSKNYQD,
(SEQ ID NO: 217)
EKLIETYFSKNYQDY,
(SEQ ID NO: 218)
WTTCQSIAFPSKTSA,
(SEQ ID NO: 219)
TTCQSIAFPSKTSAS,
(SEQ ID NO: 220)
TCQSIAFPSKTSASI,
(SEQ ID NO: 221)
CQSIAFPSKTSASIG,
(SEQ ID NO: 222)
QSIAFPSKTSASIGS,
(SEQ ID NO: 223)
VLPWNAFPGKVCGSN,
(SEQ ID NO: 224)
HEALTGTEKLIETYFSKNYQDYEYLI,
(SEQ ID NO: 225)
TWTTCQSIAFPSKTSASIGSLCADARMY,
and
(SEQ ID NO: 226)
GVLPWNAFPGKVCGSNLLSICKTAEFQM,
or combinations thereof;
wherein said immunogenic peptide comprises a T-cell epitope derived from the myelin basic protein (MBP) antigen amino acid sequence preferably selected from the group comprising: PRHRDTGILDSIGRF (SEQ ID NO: 227)
(SEQ ID NO: 228)
ENPVVHFFKNIVTPRTP
(SEQ ID NO: 229)
RASDYKSAHKGFKGV
(SEQ ID NO: 230)
GFKGVDAQGTLSKIF
(SEQ ID NO: 231)
LGGRDSRSGSPMARR
(SEQ ID NO: 232)
TQDENPVVHFFKNIVTPRTP
(SEQ ID NO: 233)
TQDENPVVHFFKNIV
(SEQ ID NO: 234)
QDENPVVHFFKNIVT
(SEQ ID NO: 235)
DENPVVHFFKNIVTP
(SEQ ID NO: 236)
ENPVVHFFKNIVTPR
(SEQ ID NO: 237)
NPVVHFFKNIVTPRT
(SEQ ID NO: 238)
PVVHFFKNIVTPRTP
(SEQ ID NO: 239)
ASDYKSAHKGFKGVDAQGTLSKIFKLGG
(SEQ ID NO: 240)
ASDYKSAHKGFKGVD
(SEQ ID NO: 241)
SDYKSAHKGFKGVDA
(SEQ ID NO: 242)
DYKSAHKGFKGVDAQ
(SEQ ID NO: 243)
YKSAHKGFKGVDAQG
(SEQ ID NO: 244)
KSAHKGFKGVDAQGT
(SEQ ID NO: 245)
SAHKGFKGVDAQGTL
(SEQ ID NO: 246)
AHKGFKGVDAQGTLS
(SEQ ID NO: 247)
HKGFKGVDAQGTLSK
(SEQ ID NO: 248)
KGFKGVDAQGTLSKI
(SEQ ID NO: 249)
GFKGVDAQGTLSKIF
(SEQ ID NO: 250)
FKGVDAQGTLSKIFK
(SEQ ID NO: 251)
KGVDAQGTLSKIFKL
(SEQ ID NO: 252)
GVDAQGTLSKIFKLG
(SEQ ID NO: 253)
VDAQGTLSKIFKLGG,
and
(SEQ ID NO: 254)
LSRFSWGAEGQRPG,
or combinations thereof, preferable a cocktail of all 4 peptides defined in SEQ ID NO: 227 to 230.

16. The pharmaceutical kit according to any one of claims 1 to 15, wherein said immunogenic or tolerogenic peptide comprises a T-cell epitope derived from the Myelin-oligodendrocyte glycoprotein (MOG) antigen amino acid sequence selected from YRSPFSRVV (SEQ ID NO: 169) and YRPPFSRVV (human SEQ ID NO: 168), and comprises as a linker the amino acid sequence GW and comprises as a flanker the amino acid sequence HLYR (SEQ ID NO: 186).

17. The pharmaceutical kit according to any one of claims 1 to 16, wherein said immunogenic or tolerogenic peptide comprises a T-cell epitope derived from the Myelin-oligodendrocyte glycoprotein (MOG) antigen amino acid sequence selected from FLRVPCWKI (SEQ ID NO:164), and FLRVPSWKI (SEQ ID NO:165), and comprises as a linker the amino acid sequence VRY (SEQ ID NO: 271) and comprises as a flanker an amino acid sequence selected from: TLF (SEQ ID NO: 269), TLFK (SEQ ID NO: 270), or TLFKK (SEQ ID NO: 267).

18. The pharmaceutical kit according to any one of claims 2 to 15, wherein said immunogenic peptide is selected from the group consisting of:
(SEQ ID NO: 176)
KHCPYCVRYFLRVPSWKITLFKK,
(SEQ ID NO: 177)
KHCPYCVRYFLRVPCWKITLFKK.
(SEQ ID NO: 174)
HCPYCVRYFLRVPSWKITLF,
(SEQ ID NO: 175)
HCPYCVRYFLRVPCWKITLF,
(SEQ ID NO: 185)
HCPYCGWYRSPFSRVVHLYR,
(SEQ ID NO: 266)
KCRCGWYRSPFSRVVHLYR,
and
(SEQ ID NO: 268)
KCRPYCGWYRSPFSRVVHLYR.

19. The pharmaceutical kit according to any one of aspects 1 to 18, wherein said immunogenic or tolerogenic peptides are present in the form of one or more nucleic acid molecules encoding said immunogenic or tolerogenic peptides, preferably wherein said nucleic acid is selected from: isolated desoxyribonucleic acid (DNA), plasmid DNA (pDNA), coding DNA (cDNA), ribonucleic acid (RNA), messenger RNA (mRNA) or modified versions thereof.

20. The pharmaceutical kit according to any one of claims 1 to 19, for use in treating, preventing and/or ameliorating symptoms of a fumarate-related disease or disorder.

21. The pharmaceutical kit for use according to claim 20, for use in treating, preventing and/or ameliorating symptoms of auto-immune disorders such as psoriasis, Rheumatoid Arthritis (RA), asthma, atopic dermatitis, scleroderma, ulcerative colitis; demyelinating disorders such as Multiple Sclerosis (MS), Neuromyelitis Optica (NMO); transplant rejection; or cancer.

22. The pharmaceutical kit for use according to claim 20 or 21, wherein said immunogenic or tolerogenic peptide is administered to the subject as a peptide or as a nucleic acid encoding said immunogenic or tolerogenic peptide, preferably selected from isolated desoxyribonucleic acid (DNA), plasmid DNA (pDNA), coding DNA (cDNA), ribonucleic acid (RNA), messenger RNA (mRNA) or modified versions thereof.

23. A method of treatment of, ameliorating the symptoms of, and/or preventing of a fumarate-related disease or disorder in a patient in need thereof, comprising the step of administering an effective amount of the pharmaceutical kit according to any one of aspects 1 to 19.

24. The pharmaceutical kit for use according to claim 21, 22, or 23, or the method according to claim 23, wherein said fumarate compound and said immunogenic or tolerogenic peptide are administered simultaneously, sequentially and/or separately.

25. The pharmaceutical kit for use according to claim 21, 22, or 23, or the method according to claim 23 or 24, wherein said fumarate compound is administered orally once or twice per day, and/or wherein said immunogenic or tolerogenic peptide is administered through subcutaneous injection.


SEQUENCE LISTING (255 sequences)

SEQ ID NO: 1 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 2 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 3 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 4 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 5 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 6 - protein, 4 aa, Artificial Sequence
  synthetic peptide
  misc_feature (1)..(1): Xaa can be any naturally occurring amino acid
  PEPTIDE (2)..(2): C, S, or T
  misc_feature (3)..(3): Xaa can be any naturally occurring amino acid
XXXC

SEQ ID NO: 7 - protein, 5 aa, Artificial Sequence
  synthetic peptide
  misc_feature (1)..(1): Xaa can be any naturally occurring amino acid
  PEPTIDE (2)..(2): C, S, or T
  misc_feature (3)..(4): Xaa can be any naturally occurring amino acid
XXXXC

SEQ ID NO: 8 - protein, 6 aa, Artificial Sequence
  synthetic peptide
  misc_feature (1)..(1): Xaa can be any naturally occurring amino acid
  PEPTIDE (2)..(2): C, S, or T
  misc_feature (3)..(5): Xaa can be any naturally occurring amino acid
XXXXXC

SEQ ID NO: 9 - protein, 7 aa, Artificial Sequence
  synthetic peptide
  misc_feature (1)..(1): Xaa can be any naturally occurring amino acid
  PEPTIDE (2)..(2): C, S, or T
  misc_feature (3)..(6): Xaa can be any naturally occurring amino acid
XXXXXXC

SEQ ID NO: 10 - protein, 8 aa, Artificial Sequence
  synthetic peptide
  misc_feature (1)..(1): Xaa can be any naturally occurring amino acid
  PEPTIDE (2)..(2): C, S, or T
  misc_feature (3)..(7): Xaa can be any naturally occurring amino acid
XXXXXXXC

SEQ ID NO: 11 - protein, 9 aa, Artificial Sequence
  synthetic peptide
  misc_feature (1)..(1): Xaa can be any naturally occurring amino acid
  PEPTIDE (2)..(2): C, S, or T
  misc_feature (3)..(8): Xaa can be any naturally occurring amino acid
XXXXXXXXC

SEQ ID NO: 12 - protein, 4 aa, Artificial Sequence
  synthetic peptide
  misc_feature (1)..(2): Xaa can be any naturally occurring amino acid
  PEPTIDE (3)..(3): C, S, or T
XXXC

SEQ ID NO: 13 - protein, 5 aa, Artificial Sequence
  synthetic peptide
  misc_feature (1)..(2): Xaa can be any naturally occurring amino acid
  PEPTIDE (3)..(3): C, S, or T
  misc_feature (4)..(4): Xaa can be any naturally occurring amino acid
XXXXC

SEQ ID NO: 14 - protein, 6 aa, Artificial Sequence
  synthetic peptide
  misc_feature (1)..(2): Xaa can be any naturally occurring amino acid
  PEPTIDE (3)..(3): C, S, or T
  misc_feature (4)..(5): Xaa can be any naturally occurring amino acid
XXXXXC

SEQ ID NO: 15 - protein, 7 aa, Artificial Sequence
  synthetic peptide
  misc_feature (1)..(2): Xaa can be any naturally occurring amino acid
  PEPTIDE (3)..(3): C, S, or T
  misc_feature (4)..(6): Xaa can be any naturally occurring amino acid
XXXXXXC

SEQ ID NO: 16 - protein, 8 aa, Artificial Sequence
  synthetic peptide
  misc_feature (1)..(2): Xaa can be any naturally occurring amino acid
  PEPTIDE (3)..(3): C, S, or T
  misc_feature (4)..(7): Xaa can be any naturally occurring amino acid
XXXXXXXC

SEQ ID NO: 17 - protein, 9 aa, Artificial Sequence
  synthetic peptide
  misc_feature (1)..(2): Xaa can be any naturally occurring amino acid
  PEPTIDE (3)..(3): C, S, or T
  misc_feature (4)..(8): Xaa can be any naturally occurring amino acid
XXXXXXXXC

SEQ ID NO: 18 - protein, 10 aa, Artificial Sequence
  synthetic peptide
  misc_feature (1)..(2): Xaa can be any naturally occurring amino acid
  PEPTIDE (3)..(3): C, S, or T
  misc_feature (4)..(9): Xaa can be any naturally occurring amino acid
XXXXXXXXXC

SEQ ID NO: 19 - protein, 5 aa, Artificial Sequence
  synthetic peptide
  misc_feature (1)..(3): Xaa can be any naturally occurring amino acid
  PEPTIDE (4)..(4): C, S, or T
XXXXC

SEQ ID NO: 20 - protein, 6 aa, Artificial Sequence
  synthetic peptide
  misc_feature (1)..(3): Xaa can be any naturally occurring amino acid
  PEPTIDE (4)..(4): C, S, or T
  misc_feature (5)..(5): Xaa can be any naturally occurring amino acid
XXXXXC

SEQ ID NO: 21 - protein, 7 aa, Artificial Sequence
  synthetic peptide
  misc_feature (1)..(3): Xaa can be any naturally occurring amino acid
  PEPTIDE (4)..(4): C, S, or T
  misc_feature (5)..(6): Xaa can be any naturally occurring amino acid
XXXXXXC

SEQ ID NO: 22 - protein, 8 aa, Artificial Sequence
  synthetic peptide
  misc_feature (1)..(3): Xaa can be any naturally occurring amino acid
  PEPTIDE (4)..(4): C, S, or T
  misc_feature (5)..(7): Xaa can be any naturally occurring amino acid
XXXXXXXC

SEQ ID NO: 23 - protein, 9 aa, Artificial Sequence
  synthetic peptide
  misc_feature (1)..(3): Xaa can be any naturally occurring amino acid
  PEPTIDE (4)..(4): C, S, or T
  misc_feature (5)..(8): Xaa can be any naturally occurring amino acid
XXXXXXXXC

SEQ ID NO: 24 - protein, 10 aa, Artificial Sequence
  synthetic peptide
  misc_feature (1)..(3): Xaa can be any naturally occurring amino acid
  PEPTIDE (4)..(4): C, S, or T
  misc_feature (5)..(9): Xaa can be any naturally occurring amino acid
XXXXXXXXXC

SEQ ID NO: 25 - protein, 11 aa, Artificial Sequence
  synthetic peptide
  misc_feature (1)..(3): Xaa can be any naturally occurring amino acid
  PEPTIDE (4)..(4): C, S, or T
  misc_feature (5)..(10): Xaa can be any naturally occurring amino acid
XXXXXXXXXXC

SEQ ID NO: 26 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 27 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 28 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 29 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 30 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 31 - protein, 4 aa, Artificial Sequence
  synthetic peptide
  misc_feature (1)..(1): Xaa can be any naturally occurring amino acid
  misc_feature (3)..(3): Xaa can be any naturally occurring amino acid
  PEPTIDE (4)..(4): C, S, or T
XCXX

SEQ ID NO: 32 - protein, 5 aa, Artificial Sequence
  synthetic peptide
  misc_feature (1)..(1): Xaa can be any naturally occurring amino acid
  misc_feature (3)..(4): Xaa can be any naturally occurring amino acid
  PEPTIDE (5)..(5): C, S, or T
XCXXX

SEQ ID NO: 33 - protein, 6 aa, Artificial Sequence
  synthetic peptide
  misc_feature (1)..(1): Xaa can be any naturally occurring amino acid
  misc_feature (3)..(5): Xaa can be any naturally occurring amino acid
  PEPTIDE (6)..(6): C, S, or T
XCXXXX

SEQ ID NO: 34 - protein, 7 aa, Artificial Sequence
  synthetic peptide
  misc_feature (1)..(1): Xaa can be any naturally occurring amino acid
  misc_feature (3)..(6): Xaa can be any naturally occurring amino acid
  PEPTIDE (7)..(7): C, S, or T
XCXXXXX

SEQ ID NO: 35 - protein, 8 aa, Artificial Sequence
  synthetic peptide
  misc_feature (1)..(1): Xaa can be any naturally occurring amino acid
  misc_feature (3)..(7): Xaa can be any naturally occurring amino acid
  PEPTIDE (8)..(8): C, S, or T
XCXXXXXX

SEQ ID NO: 36 - protein, 9 aa, Artificial Sequence
  synthetic peptide
  misc_feature (1)..(1): Xaa can be any naturally occurring amino acid
  misc_feature (3)..(8): Xaa can be any naturally occurring amino acid
  PEPTIDE (9)..(9): C, S, or T
XCXXXXXXX

SEQ ID NO: 37 - protein, 4 aa, Artificial Sequence
  synthetic peptide
  misc_feature (1)..(2): Xaa can be any naturally occurring amino acid
  PEPTIDE (4)..(4): C, S, or T
XXCX

SEQ ID NO: 38 - protein, 5 aa, Artificial Sequence
  synthetic peptide
  misc_feature (1)..(2): Xaa can be any naturally occurring amino acid
  misc_feature (4)..(4): Xaa can be any naturally occurring amino acid
  PEPTIDE (5)..(5): C, S, or T
XXCXX

SEQ ID NO: 39 - protein, 6 aa, Artificial Sequence
  synthetic peptide
  misc_feature (1)..(2): Xaa can be any naturally occurring amino acid
  misc_feature (4)..(5): Xaa can be any naturally occurring amino acid
  PEPTIDE (6)..(6): C, S, or T
XXCXXX

SEQ ID NO: 40 - protein, 7 aa, Artificial Sequence
  synthetic peptide
  misc_feature (1)..(2): Xaa can be any naturally occurring amino acid
  misc_feature (4)..(6): Xaa can be any naturally occurring amino acid
  PEPTIDE (7)..(7): C, S, or T
XXCXXXX

SEQ ID NO: 41 - protein, 8 aa, Artificial Sequence
  synthetic peptide
  misc_feature (1)..(2): Xaa can be any naturally occurring amino acid
  misc_feature (4)..(7): Xaa can be any naturally occurring amino acid
  PEPTIDE (8)..(8): C, S, or T
XXCXXXXX

SEQ ID NO: 42 - protein, 9 aa, Artificial Sequence
  synthetic peptide
  misc_feature (1)..(2): Xaa can be any naturally occurring amino acid
  misc_feature (4)..(8): Xaa can be any naturally occurring amino acid
  PEPTIDE (9)..(9): C, S, or T
XXCXXXXXX

SEQ ID NO: 43 - protein, 10 aa, Artificial Sequence
  synthetic peptide
  misc_feature (1)..(2): Xaa can be any naturally occurring amino acid
  misc_feature (4)..(9): Xaa can be any naturally occurring amino acid
  PEPTIDE (10)..(10): C, S, or T
XXCXXXXXXX

SEQ ID NO: 44 - protein, 5 aa, Artificial Sequence
  synthetic peptide
  misc_feature (1)..(3): Xaa can be any naturally occurring amino acid
  PEPTIDE (5)..(5): C, S, or T
XXXCX

SEQ ID NO: 45 - protein, 6 aa, Artificial Sequence
  synthetic peptide
  misc_feature (1)..(3): Xaa can be any naturally occurring amino acid
  misc_feature (5)..(5): Xaa can be any naturally occurring amino acid
  PEPTIDE (6)..(6): C, S, or T
XXXCXX

SEQ ID NO: 46 - protein, 7 aa, Artificial Sequence
  synthetic peptide
  misc_feature (1)..(3): Xaa can be any naturally occurring amino acid
  misc_feature (5)..(6): Xaa can be any naturally occurring amino acid
  PEPTIDE (7)..(7): C, S, or T
XXXCXXX

SEQ ID NO: 47 - protein, 8 aa, Artificial Sequence
  synthetic peptide
  misc_feature (1)..(3): Xaa can be any naturally occurring amino acid
  misc_feature (5)..(7): Xaa can be any naturally occurring amino acid
  PEPTIDE (8)..(8): C, S, or T
XXXCXXXX

SEQ ID NO: 48 - protein, 9 aa, Artificial Sequence
  synthetic peptide
  misc_feature (1)..(3): Xaa can be any naturally occurring amino acid
  misc_feature (5)..(8): Xaa can be any naturally occurring amino acid
  PEPTIDE (9)..(9): C, S, or T
XXXCXXXXX

SEQ ID NO: 49 - protein, 10 aa, Artificial Sequence
  synthetic peptide
  misc_feature (1)..(3): Xaa can be any naturally occurring amino acid
  misc_feature (5)..(9): Xaa can be any naturally occurring amino acid
  PEPTIDE (10)..(10): C, S, or T
XXXCXXXXXX

SEQ ID NO: 50 - protein, 11 aa, Artificial Sequence
  synthetic peptide
  misc_feature (1)..(3): Xaa can be any naturally occurring amino acid
  misc_feature (5)..(10): Xaa can be any naturally occurring amino acid
  PEPTIDE (11)..(11): C, S, or T
XXXCXXXXXXX

SEQ ID NO: 51 - protein, 7 aa, Artificial Sequence
  synthetic peptide
  PEPTIDE (1)..(1): F, W, H, or Y
  misc_feature (2)..(3): Xaa can be any naturally occurring amino acid
  PEPTIDE (4)..(4): I, L, M, or V
  misc_feature (5)..(6): Xaa can be any naturally occurring amino acid
  PEPTIDE (7)..(7): F, W, T, H, or Y
XXXXXXX

SEQ ID NO: 52 - protein, 7 aa, Artificial Sequence
  synthetic peptide
  PEPTIDE (1)..(1): F or W
  misc_feature (2)..(3): Xaa can be any naturally occurring amino acid
  PEPTIDE (4)..(4): I, L, or M
  misc_feature (5)..(6): Xaa can be any naturally occurring amino acid
  PEPTIDE (7)..(7): F or W
XXXXXXX

SEQ ID NO: 53 - protein, 4 aa, Artificial Sequence
  synthetic peptide
KKCC

SEQ ID NO: 54 - protein, 4 aa, Artificial Sequence
  synthetic peptide
RRCC

SEQ ID NO: 55 - protein, 4 aa, Artificial Sequence
  synthetic peptide
RKCC

SEQ ID NO: 56 - protein, 4 aa, Artificial Sequence
  synthetic peptide
KRCC

SEQ ID NO: 57 - protein, 4 aa, Artificial Sequence
  synthetic peptide
  misc_feature (3)..(3): Xaa can be any naturally occurring amino acid
KCXC

SEQ ID NO: 58 - protein, 5 aa, Artificial Sequence
  synthetic peptide
  misc_feature (4)..(4): Xaa can be any naturally occurring amino acid
KKCXC

SEQ ID NO: 59 - protein, 4 aa, Artificial Sequence
  synthetic peptide
  misc_feature (3)..(3): Xaa can be any naturally occurring amino acid
RCXC

SEQ ID NO: 60 - protein, 5 aa, Artificial Sequence
  synthetic peptide
  misc_feature (4)..(4): Xaa can be any naturally occurring amino acid
RRCXC

SEQ ID NO: 61 - protein, 5 aa, Artificial Sequence
  synthetic peptide
  misc_feature (4)..(4): Xaa can be any naturally occurring amino acid
RKCXC

SEQ ID NO: 62 - protein, 5 aa, Artificial Sequence
  synthetic peptide
  misc_feature (4)..(4): Xaa can be any naturally occurring amino acid
KRCXC

SEQ ID NO: 63 - protein, 4 aa, Artificial Sequence
  synthetic peptide
KCKC

SEQ ID NO: 64 - protein, 5 aa, Artificial Sequence
  synthetic peptide
KKCKC

SEQ ID NO: 65 - protein, 4 aa, Artificial Sequence
  synthetic peptide
KCRC

SEQ ID NO: 66 - protein, 5 aa, Artificial Sequence
  synthetic peptide
KKCRC

SEQ ID NO: 67 - protein, 4 aa, Artificial Sequence
  synthetic peptide
RCRC

SEQ ID NO: 68 - protein, 5 aa, Artificial Sequence
  synthetic peptide
RRCRC

SEQ ID NO: 69 - protein, 5 aa, Artificial Sequence
  synthetic peptide
RKCKC

SEQ ID NO: 70 - protein, 5 aa, Artificial Sequence
  synthetic peptide
KRCKC

SEQ ID NO: 71 - protein, 5 aa, Artificial Sequence
  synthetic peptide
HCPYC

SEQ ID NO: 72 - protein, 5 aa, Artificial Sequence
  synthetic peptide
KCPYC

SEQ ID NO: 73 - protein, 5 aa, Artificial Sequence
  synthetic peptide
RCPYC

SEQ ID NO: 74 - protein, 5 aa, Artificial Sequence
  synthetic peptide
HCGHC

SEQ ID NO: 75 - protein, 5 aa, Artificial Sequence
  synthetic peptide
KCGHC

SEQ ID NO: 76 - protein, 5 aa, Artificial Sequence
  synthetic peptide
RCGHC

SEQ ID NO: 77 - protein, 6 aa, Artificial Sequence
  synthetic peptide
KHCPYC

SEQ ID NO: 78 - protein, 6 aa, Artificial Sequence
  synthetic peptide
KKCPYC

SEQ ID NO: 79 - protein, 6 aa, Artificial Sequence
  synthetic peptide
KRCPYC

SEQ ID NO: 80 - protein, 6 aa, Artificial Sequence
  synthetic peptide
KHCGHC

SEQ ID NO: 81 - protein, 6 aa, Artificial Sequence
  synthetic peptide
KKCGHC

SEQ ID NO: 82 - protein, 6 aa, Artificial Sequence
  synthetic peptide
KRCGHC

SEQ ID NO: 83 - protein, 5 aa, Artificial Sequence
  synthetic peptide
CRPYC

SEQ ID NO: 84 - protein, 6 aa, Artificial Sequence
  synthetic peptide
KCRPYC

SEQ ID NO: 85 - protein, 7 aa, Artificial Sequence
  synthetic peptide
KHCRPYC

SEQ ID NO: 86 - protein, 6 aa, Artificial Sequence
  synthetic peptide
RCRPYC

SEQ ID NO: 87 - protein, 6 aa, Artificial Sequence
  synthetic peptide
HCRPYC

SEQ ID NO: 88 - protein, 5 aa, Artificial Sequence
  synthetic peptide
CPRYC

SEQ ID NO: 89 - protein, 6 aa, Artificial Sequence
  synthetic peptide
KCPRYC

SEQ ID NO: 90 - protein, 6 aa, Artificial Sequence
  synthetic peptide
RCPRYC

SEQ ID NO: 91 - protein, 6 aa, Artificial Sequence
  synthetic peptide
HCPRYC

SEQ ID NO: 92 - protein, 5 aa, Artificial Sequence
  synthetic peptide
CPYRC

SEQ ID NO: 93 - protein, 6 aa, Artificial Sequence
  synthetic peptide
KCPYRC

SEQ ID NO: 94 - protein, 6 aa, Artificial Sequence
  synthetic peptide
RCPYRC

SEQ ID NO: 95 - protein, 6 aa, Artificial Sequence
  synthetic peptide
HCPYRC

SEQ ID NO: 96 - protein, 5 aa, Artificial Sequence
  synthetic peptide
CKPYC

SEQ ID NO: 97 - protein, 6 aa, Artificial Sequence
  synthetic peptide
KCKPYC

SEQ ID NO: 98 - protein, 6 aa, Artificial Sequence
  synthetic peptide
RCKPYC

SEQ ID NO: 99 - protein, 6 aa, Artificial Sequence
  synthetic peptide
HCKPYC

SEQ ID NO: 100 - protein, 5 aa, Artificial Sequence
  synthetic peptide
CPKYC

SEQ ID NO: 101 - protein, 6 aa, Artificial Sequence
  synthetic peptide
KCPKYC

SEQ ID NO: 102 - protein, 6 aa, Artificial Sequence
  synthetic peptide
RCPKYC

SEQ ID NO: 103 - protein, 6 aa, Artificial Sequence
  synthetic peptide
HCPKYC

SEQ ID NO: 104 - protein, 5 aa, Artificial Sequence
  synthetic peptide
CPYKC

SEQ ID NO: 105 - protein, 6 aa, Artificial Sequence
  synthetic peptide
KCPYKC

SEQ ID NO: 106 - protein, 6 aa, Artificial Sequence
  synthetic peptide
RCPYKC

SEQ ID NO: 107 - protein, 6 aa, Artificial Sequence
  synthetic peptide
HCPYKC

SEQ ID NO: 108 - protein, 4 aa, Artificial Sequence
  synthetic peptide
LAVL

SEQ ID NO: 109 - protein, 4 aa, Artificial Sequence
  synthetic peptide
TVQA

SEQ ID NO: 110 - protein, 4 aa, Artificial Sequence
  synthetic peptide
GAVH

SEQ ID NO: 111 - protein, 4 aa, Artificial Sequence
  synthetic peptide
  misc_feature (1)..(4): Xaa can be any naturally occurring amino acid
XXXX

SEQ ID NO: 112 - protein, 4 aa, Artificial Sequence
  synthetic peptide
  misc_feature (1)..(1): Xaa can be any naturally occurring amino acid
XAVL

SEQ ID NO: 113 - protein, 4 aa, Artificial Sequence
  synthetic peptide
  misc_feature (2)..(2): Xaa can be any naturally occurring amino acid
LXVL

SEQ ID NO: 114 - protein, 4 aa, Artificial Sequence
  synthetic peptide
  misc_feature (3)..(3): Xaa can be any naturally occurring amino acid
LAXL

SEQ ID NO: 115 - protein, 4 aa, Artificial Sequence
  synthetic peptide
  misc_feature (4)..(4): Xaa can be any naturally occurring amino acid
LAVX

SEQ ID NO: 116 - protein, 4 aa, Artificial Sequence
  synthetic peptide
  misc_feature (1)..(1): Xaa can be any naturally occurring amino acid
XVQA

SEQ ID NO: 117 - protein, 4 aa, Artificial Sequence
  synthetic peptide
  misc_feature (2)..(2): Xaa can be any naturally occurring amino acid
TXQA

SEQ ID NO: 118 - protein, 4 aa, Artificial Sequence
  synthetic peptide
  misc_feature (4)..(4): Xaa can be any naturally occurring amino acid
TVQX

SEQ ID NO: 119 - protein, 4 aa, Artificial Sequence
  synthetic peptide
  misc_feature (1)..(1): Xaa can be any naturally occurring amino acid
XAVH

SEQ ID NO: 120 - protein, 4 aa, Artificial Sequence
  synthetic peptide
  misc_feature (2)..(2): Xaa can be any naturally occurring amino acid
GXVH

SEQ ID NO: 121 - protein, 4 aa, Artificial Sequence
  synthetic peptide
  misc_feature (3)..(3): Xaa can be any naturally occurring amino acid
GAXH

SEQ ID NO: 122 - protein, 4 aa, Artificial Sequence
  synthetic peptide
  misc_feature (4)..(4): Xaa can be any naturally occurring amino acid
GAVX

SEQ ID NO: 123 - protein, 5 aa, Artificial Sequence
  synthetic peptide
PAFPL

SEQ ID NO: 124 - protein, 5 aa, Artificial Sequence
  synthetic peptide
DQGGE

SEQ ID NO: 125 - protein, 5 aa, Artificial Sequence
  synthetic peptide
  misc_feature (1)..(5): Xaa can be any naturally occurring amino acid
XXXXX

SEQ ID NO: 126 - protein, 5 aa, Artificial Sequence
  synthetic peptide
  misc_feature (1)..(1): Xaa can be any naturally occurring amino acid
XAFPL

SEQ ID NO: 127 - protein, 5 aa, Artificial Sequence
  synthetic peptide
  misc_feature (2)..(2): Xaa can be any naturally occurring amino acid
PXFPL

SEQ ID NO: 128 - protein, 5 aa, Artificial Sequence
  synthetic peptide
  misc_feature (3)..(3): Xaa can be any naturally occurring amino acid
PAXPL

SEQ ID NO: 129 - protein, 5 aa, Artificial Sequence
  synthetic peptide
  misc_feature (4)..(4): Xaa can be any naturally occurring amino acid
PAFXL

SEQ ID NO: 130 - protein, 5 aa, Artificial Sequence
  synthetic peptide
  misc_feature (5)..(5): Xaa can be any naturally occurring amino acid
PAFPX

SEQ ID NO: 131 - protein, 5 aa, Artificial Sequence
  synthetic peptide
  misc_feature (1)..(1): Xaa can be any naturally occurring amino acid
XQGGE

SEQ ID NO: 132 - protein, 5 aa, Artificial Sequence
  synthetic peptide
  misc_feature (2)..(2): Xaa can be any naturally occurring amino acid
DXGGE

SEQ ID NO: 133 - protein, 5 aa, Artificial Sequence
  synthetic peptide
  misc_feature (3)..(3): Xaa can be any naturally occurring amino acid
DQXGE

SEQ ID NO: 134 - protein, 5 aa, Artificial Sequence
  synthetic peptide
  misc_feature (4)..(4): Xaa can be any naturally occurring amino acid
DQGXE

SEQ ID NO: 135 - protein, 5 aa, Artificial Sequence
  synthetic peptide
  misc_feature (5)..(5): Xaa can be any naturally occurring amino acid
DQGGX

SEQ ID NO: 136 - protein, 6 aa, Artificial Sequence
  synthetic peptide
DIADKY

SEQ ID NO: 137 - protein, 6 aa, Artificial Sequence
  synthetic peptide
  misc_feature (1)..(6): Xaa can be any naturally occurring amino acid
XXXXXX

SEQ ID NO: 138 - protein, 6 aa, Artificial Sequence
  synthetic peptide
  misc_feature (1)..(1): Xaa can be any naturally occurring amino acid
XIADKY

SEQ ID NO: 139 - protein, 6 aa, Artificial Sequence
  synthetic peptide
  misc_feature (2)..(2): Xaa can be any naturally occurring amino acid
DXADKY

SEQ ID NO: 140 - protein, 6 aa, Artificial Sequence
  synthetic peptide
  misc_feature (3)..(3): Xaa can be any naturally occurring amino acid
DIXDKY

SEQ ID NO: 141 - protein, 6 aa, Artificial Sequence
  synthetic peptide
  misc_feature (4)..(4): Xaa can be any naturally occurring amino acid
DIAXKY

SEQ ID NO: 142 - protein, 6 aa, Artificial Sequence
  synthetic peptide
  misc_feature (5)..(5): Xaa can be any naturally occurring amino acid
DIADXY

SEQ ID NO: 143 - protein, 6 aa, Artificial Sequence
  synthetic peptide
  misc_feature (6)..(6): Xaa can be any naturally occurring amino acid
DIADKX

SEQ ID NO: 144 - protein, 4 aa, Artificial Sequence
  synthetic peptide
  PEPTIDE (1)..(1): acetylated, methylated, ethylated, or propionylated C, S, or T
  misc_feature (2)..(3): Xaa can be any naturally occurring amino acid
XXXC

SEQ ID NO: 145 - protein, 5 aa, Artificial Sequence
  synthetic peptide
  PEPTIDE (1)..(1): acetylated, methylated, ethylated, or propionylated C, S, or T
  misc_feature (2)..(4): Xaa can be any naturally occurring amino acid
XXXXC

SEQ ID NO: 146 - protein, 6 aa, Artificial Sequence
  synthetic peptide
  PEPTIDE (1)..(1): acetylated, methylated, ethylated, or propionylated C, S, or T
  misc_feature (2)..(5): Xaa can be any naturally occurring amino acid
XXXXXC

SEQ ID NO: 147 - protein, 7 aa, Artificial Sequence
  synthetic peptide
  PEPTIDE (1)..(1): acetylated, methylated, ethylated, or propionylated C, S, or T
  misc_feature (2)..(6): Xaa can be any naturally occurring amino acid
XXXXXXC

SEQ ID NO: 148 - protein, 8 aa, Artificial Sequence
  synthetic peptide
  PEPTIDE (1)..(1): acetylated, methylated, ethylated, or propionylated C, S, or T
  misc_feature (2)..(7): Xaa can be any naturally occurring amino acid
XXXXXXXC

SEQ ID NO: 149 - protein, 4 aa, Artificial Sequence
  synthetic peptide
  misc_feature (2)..(3): Xaa can be any naturally occurring amino acid
  PEPTIDE (4)..(4): acetylated, methylated, ethylated, or propionylated C, S, or T
CXXX

SEQ ID NO: 150 - protein, 5 aa, Artificial Sequence
  synthetic peptide
  misc_feature (2)..(4): Xaa can be any naturally occurring amino acid
  PEPTIDE (5)..(5): acetylated, methylated, ethylated, or propionylated C, S, or T
CXXXX

SEQ ID NO: 151 - protein, 6 aa, Artificial Sequence
  synthetic peptide
  misc_feature (2)..(5): Xaa can be any naturally occurring amino acid
  PEPTIDE (6)..(6): acetylated, methylated, ethylated, or propionylated C, S, or T
CXXXXX

SEQ ID NO: 152 - protein, 7 aa, Artificial Sequence
  synthetic peptide
  misc_feature (2)..(6): Xaa can be any naturally occurring amino acid
  PEPTIDE (7)..(7): acetylated, methylated, ethylated, or propionylated C, S, or T
CXXXXXX

SEQ ID NO: 153 - protein, 8 aa, Artificial Sequence
  synthetic peptide
  misc_feature (2)..(7): Xaa can be any naturally occurring amino acid
  PEPTIDE (8)..(8): acetylated, methylated, ethylated, or propionylated C, S, or T
CXXXXXXX

SEQ ID NO: 154 - protein, 4 aa, Artificial Sequence
  synthetic peptide
  PEPTIDE (1)..(1): C, S, or T
  misc_feature (2)..(3): Xaa can be any naturally occurring amino acid
  PEPTIDE (4)..(4): acetylated, methylated, ethylated, or propionylated C
XXXX

SEQ ID NO: 155 - protein, 5 aa, Artificial Sequence
  synthetic peptide
  PEPTIDE (1)..(1): C, S, or T
  misc_feature (2)..(4): Xaa can be any naturally occurring amino acid
  PEPTIDE (5)..(5): acetylated, methylated, ethylated, or propionylated C
XXXXX

SEQ ID NO: 156 - protein, 6 aa, Artificial Sequence
  synthetic peptide
  PEPTIDE (1)..(1): C, S, or T
  misc_feature (2)..(5): Xaa can be any naturally occurring amino acid
  PEPTIDE (6)..(6): acetylated, methylated, ethylated, or propionylated C
XXXXXX

SEQ ID NO: 157 - protein, 7 aa, Artificial Sequence
  synthetic peptide
  PEPTIDE (1)..(1): C, S, or T
  misc_feature (2)..(6): Xaa can be any naturally occurring amino acid
  PEPTIDE (7)..(7): acetylated, methylated, ethylated, or propionylated C
XXXXXXX

SEQ ID NO: 158 - protein, 8 aa, Artificial Sequence
  synthetic peptide
  PEPTIDE (1)..(1): C, S, or T
  misc_feature (2)..(7): Xaa can be any naturally occurring amino acid
  PEPTIDE (8)..(8): acetylated, methylated, ethylated, or propionylated C
XXXXXXXX

SEQ ID NO: 159 - protein, 4 aa, Artificial Sequence
  synthetic peptide
  PEPTIDE (1)..(1): acetylated, methylated, ethylated, or propionylated C
  misc_feature (2)..(3): Xaa can be any naturally occurring amino acid
  PEPTIDE (4)..(4): C, S, or T
XXXX

SEQ ID NO: 160 - protein, 5 aa, Artificial Sequence
  synthetic peptide
  PEPTIDE (1)..(1): acetylated, methylated, ethylated, or propionylated C
  misc_feature (2)..(4): Xaa can be any naturally occurring amino acid
  PEPTIDE (5)..(5): C, S, or T
XXXXX

SEQ ID NO: 161 - protein, 6 aa, Artificial Sequence
  synthetic peptide
  PEPTIDE (1)..(1): acetylated, methylated, ethylated, or propionylated C
  misc_feature (2)..(5): Xaa can be any naturally occurring amino acid
  PEPTIDE (6)..(6): C, S, or T
XXXXXX

SEQ ID NO: 162 - protein, 7 aa, Artificial Sequence
  synthetic peptide
  PEPTIDE (1)..(1): acetylated, methylated, ethylated, or propionylated C
  misc_feature (2)..(6): Xaa can be any naturally occurring amino acid
  PEPTIDE (7)..(7): C, S, or T
XXXXXXX

SEQ ID NO: 163 - protein, 8 aa, Artificial Sequence
  synthetic peptide
  PEPTIDE (1)..(1): acetylated, methylated, ethylated, or propionylated C
  misc_feature (2)..(7): Xaa can be any naturally occurring amino acid
  PEPTIDE (8)..(8): C, S, or T
XXXXXXXX

SEQ ID NO: 164 - protein, 9 aa, Homo sapiens
FLRVPCWKI

SEQ ID NO: 165 - protein, 9 aa, Artificial Sequence
  synthetic peptide
FLRVPSWKI

SEQ ID NO: 166 - protein, 12 aa, Homo sapiens
FLRVPCWKITLF

SEQ ID NO: 167 - protein, 12 aa, Artificial Sequence
  synthetic peptide
FLRVPSWKITLF

SEQ ID NO: 168 - protein, 9 aa, Homo sapiens
YRPPFSRVV

SEQ ID NO: 169 - protein, 9 aa, Mus musculus
YRSPFSRVV

SEQ ID NO: 170 - protein, 9 aa, Homo sapiens
VVHLYRNGK

SEQ ID NO: 171 - protein, 4 aa, Artificial Sequence
  synthetic peptide
RCKC

SEQ ID NO: 172 - protein, 4 aa, Artificial Sequence
  synthetic peptide
CPYC

SEQ ID NO: 173 - protein, 5 aa, Artificial Sequence
  synthetic peptide
CPRYC

SEQ ID NO: 174 - protein, 20 aa, Artificial Sequence
  synthetic peptide
HCPYCVRYFLRVPSWKITLF

SEQ ID NO: 175 - protein, 20 aa, Artificial Sequence
  synthetic peptide
HCPYCVRYFLRVPCWKITLF

SEQ ID NO: 176 - protein, 23 aa, Artificial Sequence
  synthetic peptide
KHCPYCVRYFLRVPSWKITLFKK

SEQ ID NO: 177 - protein, 23 aa, Artificial Sequence
  synthetic peptide
KHCPYCVRYFLRVPCWKITLFKK

SEQ ID NO: 178 - protein, 4 aa, Artificial Sequence
  synthetic peptide
  PEPTIDE (1)..(1): basic amino acid
  PEPTIDE (2)..(2): C, S, or T
  misc_feature (3)..(3): Xaa can be any naturally occurring amino acid
XXXC

SEQ ID NO: 179 - protein, 4 aa, Artificial Sequence
  synthetic peptide
  PEPTIDE (1)..(1): basic amino acid
  misc_feature (3)..(3): Xaa can be any naturally occurring amino acid
  PEPTIDE (4)..(4): C, S, or T
XCXX

SEQ ID NO: 180 - protein, 5 aa, Artificial Sequence
  synthetic peptide
  PEPTIDE (1)..(1): basic amino acid
  PEPTIDE (2)..(2): C, S, or T
  misc_feature (3)..(4): Xaa can be any naturally occurring amino acid
XXXXC

SEQ ID NO: 181 - protein, 5 aa, Artificial Sequence
  synthetic peptide
  PEPTIDE (1)..(1): basic amino acid
  misc_feature (3)..(4): Xaa can be any naturally occurring amino acid
  PEPTIDE (5)..(5): C, S, or T
XCXXX

SEQ ID NO: 182 - protein, 6 aa, Artificial Sequence
  synthetic peptide
  PEPTIDE (1)..(1): basic amino acid
  PEPTIDE (2)..(2): C, S, or T
  misc_feature (3)..(5): Xaa can be any naturally occurring amino acid
XXXXXC

SEQ ID NO: 183 - protein, 6 aa, Artificial Sequence
  synthetic peptide
  PEPTIDE (1)..(1): basic amino acid
  misc_feature (3)..(5): Xaa can be any naturally occurring amino acid
  PEPTIDE (6)..(6): C, S, or T
XCXXXX

SEQ ID NO: 184 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 185 - protein, 20 aa, Artificial Sequence
  synthetic peptide
HCPYCGWYRSPFSRVVHLYR

SEQ ID NO: 186 - protein, 4 aa, Artificial Sequence
  synthetic peptide
HLYR

SEQ ID NO: 187 - protein, 4 aa, Artificial Sequence
  synthetic peptide
  misc_feature (2)..(3): Xaa can be any naturally occurring amino acid
CXXC

SEQ ID NO: 188 - protein, 4 aa, Artificial Sequence
  synthetic peptide
  misc_feature (2)..(3): Xaa can be any naturally occurring amino acid
CXXS

SEQ ID NO: 189 - protein, 4 aa, Artificial Sequence
  synthetic peptide
  misc_feature (2)..(3): Xaa can be any naturally occurring amino acid
CXXT

SEQ ID NO: 190 - protein, 4 aa, Artificial Sequence
  synthetic peptide
  misc_feature (2)..(3): Xaa can be any naturally occurring amino acid
SXXC

SEQ ID NO: 191 - protein, 4 aa, Artificial Sequence
  synthetic peptide
  misc_feature (2)..(3): Xaa can be any naturally occurring amino acid
TXXC

SEQ ID NO: 192 - protein, 6 aa, Artificial Sequence
  synthetic peptide
  PEPTIDE (1)..(1): D or E
  misc_feature (2)..(4): Xaa can be any naturally occurring amino acid
  PEPTIDE (6)..(6): L or I
XXXXLX

SEQ ID NO: 193 - protein, 5 aa, Artificial Sequence
  synthetic peptide
  misc_feature (2)..(3): Xaa can be any naturally occurring amino acid
DXXLL

SEQ ID NO: 194 - protein, 6 aa, Artificial Sequence
  synthetic peptide
  misc_feature (2)..(4): Xaa can be any naturally occurring amino acid
DXXXLL

SEQ ID NO: 195 - protein, 4 aa, Artificial Sequence
  synthetic peptide
  misc_feature (2)..(3): Xaa can be any naturally occurring amino acid
  PEPTIDE (4)..(4): F, Y, or W
YXXX

SEQ ID NO: 196 - protein, 14 aa, Artificial Sequence
  synthetic peptide
  misc_feature (2)..(3): Xaa can be any naturally occurring amino acid
  misc_feature (5)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(13): Xaa can be any naturally occurring amino acid
CXXCXCXXCXCXXC

SEQ ID NO: 197 - protein, 12 aa, Artificial Sequence
  synthetic peptide
  misc_feature (2)..(3): Xaa can be any naturally occurring amino acid
  misc_feature (6)..(7): Xaa can be any naturally occurring amino acid
  misc_feature (10)..(11): Xaa can be any naturally occurring amino acid
CXXCCXXCCXXC

SEQ ID NO: 198 - protein, 10 aa, Artificial Sequence
  synthetic peptide
  misc_feature (2)..(3): Xaa can be any naturally occurring amino acid
  misc_feature (5)..(6): Xaa can be any naturally occurring amino acid
  misc_feature (8)..(9): Xaa can be any naturally occurring amino acid
CXXCXXCXXC

SEQ ID NO: 199 - protein, 10 aa, Artificial Sequence
  synthetic peptide
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
CXCCXCCXCC

SEQ ID NO: 200 - protein, 21 aa, Artificial Sequence
  synthetic peptide
YRPPFSRVVHLYRNGKDQDGD

SEQ ID NO: 201 - protein, 15 aa, Artificial Sequence
  synthetic peptide
RPPFSRVVHLYRNGK

SEQ ID NO: 202 - protein, 15 aa, Artificial Sequence
  synthetic peptide
PFSRVVHLYRNGKDQ

SEQ ID NO: 203 - protein, 15 aa, Artificial Sequence
  synthetic peptide
FSRVVHLYRNGKDQD

SEQ ID NO: 204 - protein, 15 aa, Artificial Sequence
  synthetic peptide
SRVVHLYRNGKDQDG

SEQ ID NO: 205 - protein, 21 aa, Artificial Sequence
  synthetic peptide
MEVGWYRSPFSRVVHLYRNGK

SEQ ID NO: 206 - protein, 21 aa, Artificial Sequence
  synthetic peptide
MEVGWYRPPFSRVVHLYRNGK

SEQ ID NO: 207 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 208 - protein, 29 aa, Homo sapiens
MASLSRPSLPSCLCSFLLLLLLQVSSSYA

SEQ ID NO: 209 - protein, 26 aa, Artificial Sequence
  synthetic peptide
HEALTGTEKLIETYFSKNYQDYEYLI

SEQ ID NO: 210 - protein, 28 aa, Artificial Sequence
  synthetic peptide
TWTTCQSIAFPSKTSASIGSLCADARMY

SEQ ID NO: 211 - protein, 28 aa, Artificial Sequence
  synthetic peptide
GVLPWNAFPGKVCGSNLLSICKTAEFQM

SEQ ID NO: 212 - protein, 19 aa, Artificial Sequence
  synthetic peptide
LTGTEKLIETYFSKNYQDY

SEQ ID NO: 213 - protein, 19 aa, Artificial Sequence
  synthetic peptide
WTTCQSIAFPSKTSASIGS

SEQ ID NO: 214 - protein, 15 aa, Artificial Sequence
  synthetic peptide
VLPWNAFPGKVCGSN

SEQ ID NO: 215 - protein, 15 aa, Artificial Sequence
  synthetic peptide
LTGTEKLIETYFSKN

SEQ ID NO: 216 - protein, 15 aa, Artificial Sequence
  synthetic peptide
TEKLIETYFSKNYQD

SEQ ID NO: 217 - protein, 15 aa, Artificial Sequence
  synthetic peptide
EKLIETYFSKNYQDY

SEQ ID NO: 218 - protein, 15 aa, Artificial Sequence
  synthetic peptide
WTTCQSIAFPSKTSA

SEQ ID NO: 219 - protein, 15 aa, Artificial Sequence
  synthetic peptide
TTCQSIAFPSKTSAS

SEQ ID NO: 220 - protein, 15 aa, Artificial Sequence
  synthetic peptide
TCQSIAFPSKTSASI

SEQ ID NO: 221 - protein, 15 aa, Artificial Sequence
  synthetic peptide
CQSIAFPSKTSASIG

SEQ ID NO: 222 - protein, 15 aa, Artificial Sequence
  synthetic peptide
QSIAFPSKTSASIGS

SEQ ID NO: 223 - protein, 15 aa, Artificial Sequence
  synthetic peptide
VLPWNAFPGKVCGSN

SEQ ID NO: 224 - protein, 26 aa, Artificial Sequence
  synthetic peptide
HEALTGTEKLIETYFSKNYQDYEYLI

SEQ ID NO: 225 - protein, 28 aa, Artificial Sequence
  synthetic peptide
TWTTCQSIAFPSKTSASIGSLCADARMY

SEQ ID NO: 226 - protein, 28 aa, Artificial Sequence
  synthetic peptide
GVLPWNAFPGKVCGSNLLSICKTAEFQM

SEQ ID NO: 227 - protein, 15 aa, Artificial Sequence
  synthetic peptide
PRHRDTGILDSIGRF

SEQ ID NO: 228 - protein, 17 aa, Artificial Sequence
  synthetic peptide
ENPVVHFFKNIVTPRTP

SEQ ID NO: 229 - protein, 15 aa, Artificial Sequence
  synthetic peptide
RASDYKSAHKGFKGV

SEQ ID NO: 230 - protein, 15 aa, Artificial Sequence
  synthetic peptide
GFKGVDAQGTLSKIF

SEQ ID NO: 231 - protein, 15 aa, Artificial Sequence
  synthetic peptide
LGGRDSRSGSPMARR

SEQ ID NO: 232 - protein, 20 aa, Artificial Sequence
  synthetic peptide
TQDENPVVHFFKNIVTPRTP

SEQ ID NO: 233 - protein, 15 aa, Artificial Sequence
  synthetic peptide
TQDENPVVHFFKNIV

SEQ ID NO: 234 - protein, 15 aa, Artificial Sequence
  synthetic peptide
QDENPVVHFFKNIVT

SEQ ID NO: 235 - protein, 15 aa, Artificial Sequence
  synthetic peptide
DENPVVHFFKNIVTP

SEQ ID NO: 236 - protein, 15 aa, Artificial Sequence
  synthetic peptide
ENPVVHFFKNIVTPR

SEQ ID NO: 237 - protein, 15 aa, Artificial Sequence
  synthetic peptide
NPVVHFFKNIVTPRT

SEQ ID NO: 238 - protein, 15 aa, Artificial Sequence
  synthetic peptide
PVVHFFKNIVTPRTP

SEQ ID NO: 239 - protein, 28 aa, Artificial Sequence
  synthetic peptide
ASDYKSAHKGFKGVDAQGTLSKIFKLGG

SEQ ID NO: 240 - protein, 15 aa, Artificial Sequence
  synthetic peptide
ASDYKSAHKGFKGVD

SEQ ID NO: 241 - protein, 15 aa, Artificial Sequence
  synthetic peptide
SDYKSAHKGFKGVDA

SEQ ID NO: 242 - protein, 15 aa, Artificial Sequence
  synthetic peptide
DYKSAHKGFKGVDAQ

SEQ ID NO: 243 - protein, 15 aa, Artificial Sequence
  synthetic peptide
YKSAHKGFKGVDAQG

SEQ ID NO: 244 - protein, 15 aa, Artificial Sequence
  synthetic peptide
KSAHKGFKGVDAQGT

SEQ ID NO: 245 - protein, 15 aa, Artificial Sequence
  synthetic peptide
SAHKGFKGVDAQGTL

SEQ ID NO: 246 - protein, 15 aa, Artificial Sequence
  synthetic peptide
AHKGFKGVDAQGTLS

SEQ ID NO: 247 - protein, 15 aa, Artificial Sequence
  synthetic peptide
HKGFKGVDAQGTLSK

SEQ ID NO: 248 - protein, 15 aa, Artificial Sequence
  synthetic peptide
KGFKGVDAQGTLSKI

SEQ ID NO: 249 - protein, 15 aa, Artificial Sequence
  synthetic peptide
GFKGVDAQGTLSKIF

SEQ ID NO: 250 - protein, 15 aa, Artificial Sequence
  synthetic peptide
FKGVDAQGTLSKIFK

SEQ ID NO: 251 - protein, 15 aa, Artificial Sequence
  synthetic peptide
KGVDAQGTLSKIFKL

SEQ ID NO: 252 - protein, 15 aa, Artificial Sequence
  synthetic peptide
GVDAQGTLSKIFKLG

SEQ ID NO: 253 - protein, 15 aa, Artificial Sequence
  synthetic peptide
VDAQGTLSKIFKLGG

SEQ ID NO: 254 - protein, 14 aa, Artificial Sequence
  synthetic peptide
LSRFSWGAEGQRPG

SEQ ID NO: 255 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR